(19) United States
(12) Patent Application Publication
(10) Pub. No.: US 2023/0165977 A1
(43) Pub. Date: Jun. 1, 2023

(54) MODIFIED NUCLEIC ACIDS ENCODING ASPARTOACYLASE (ASPA) AND VECTOR FOR GENE THERAPY

(71) Applicant: Pfizer Inc., New York, NY (US)

(72) Inventors: Paola Leone, Cherry Hill, NJ (US); Jeremy Francis, Cherry Hill, NJ (US); Basel T. Assaf, Dracut, MA (US); Shoh Asano, Watertown, MA (US); Kathrine Hales, Watertown, MA (US); Allison P. Berg, Cambridge, MA (US)

(73) Assignee: Pfizer Inc., New York, NY (US)

(21) Appl. No.: 17/997,312

(22) PCT Filed: Apr. 22, 2021
(86) PCT No.: PCT/US21/28658

§ 371 Date: Oct. 27, 2022

Related U.S. Application Data
(60) Provisional application No. 63/077,144, filed on Sep. 11, 2020.
(60) Provisional application No. 63/016,507, filed on Apr. 28, 2020.

(51) Int. Cl. A61K 48/00; C12N 9/80; C12N 15/86; A61P 25/28

(52) U.S. Cl. CPC A61K 48/0066; C12N 9/80; C12Y 305/01015; C12N 15/86; A61P 25/28; C12N 2750/14121; C12N 2750/14143; C12N 2750/14171; C12N 2800/22

(57) ABSTRACT

The present disclosure relates to recombinant nucleic acids and gene therapy vectors comprising a modified nucleic acid encoding aspartoacylase (ASPA), and variants thereof, for use in the treatment of diseases and disorders associated with a deficiency or dysfunction of ASPA, and in particular, Canavan disease.

### CROSS REFERENCE TO RELATED APPLICATIONS

This application claims priority to U.S. Provisional Application No. 63/016,507 filed on Apr. 28, 2020 and to U.S. Provisional Application No. 63/077,144 filed on Sep. 11, 2020. The contents of the applications are incorporated herein by reference in their entireties.

### FIELD OF THE INVENTION

The invention relates to modified nucleic acids encoding aspartoacylase (ASPA), methods of using modified nucleic acids encoding ASPA, vectors comprising modified nucleic acids encoding ASPA, and use of the vectors in the treatment of diseases, disorders and conditions associated with a decreased level of functional ASPA including diseases, disorders and conditions associated with diminished cellular catabolism of N-acetyl-L-aspartic acid, for example Canavan disease.

### BACKGROUND OF THE INVENTION

Canavan disease (CD) is associated with reduction of expression from and/or mutation of the ASPA gene that encodes the enzyme aspartoacylase (ASPA) (also known as aminoacylase 2). Decreased aspartoacylase activity results in accumulation of N-acetylaspartate (NAA) (also known as N-acetyl-L-aspartic acid) due to decreased conversion of NAA to aspartate and acetate. The ASPA enzyme has been implicated in maintenance of metabolic integrity of myelinating cells. In the brain, ASPA gene expression is restricted primarily to white matter producing oligodendrocytes. Accumulation of NAA in the brain is associated with oligodendrocyte dysfunction and interference with development of the myelin sheath and destruction of existing myelin sheath associated with neurons.

CD is an autosomal recessive genetic disease and manifests primarily in a neonatal/infantile form. Children who are affected with this form present in infancy with symptoms associated with degeneration of myelin in the brain and spinal cord. Symptoms include intellectual disability, loss of previously acquired motor skills, feeding difficulties, abnormal muscle tone, macrocephaly, paralysis and seizures. Life expectancy is generally limited to the first decade for children with the neonatal/infantile of CD. Individuals with the mild/juvenile form of CD may exhibit delayed development of speech and motor skills and have an average lifespan.

To date, no treatment exists for stopping or slowing neurodegenerative effects of CD. Current therapeutic approaches in clinical use, or under evaluation, are directed to alleviating symptoms and maximizing quality of life. Physical therapy, feeding tubes and anti-seizure medication may be used to treat some symptoms and improve quality of life. Thus, there is an important need for a novel therapeutic approach to treat CD.

### SUMMARY OF THE INVENTION

Disclosed and exemplified herein are modified nucleic acids encoding aspartoacylase (ASPA) and vectors (e.g., rAAV vector) comprising a modified nucleic acid and methods of treating a disease, disorder or condition mediated by a decreased level of ASPA protein by administering a modified nucleic acid, or a vector comprising a modified nucleic acid, to a patient in need thereof.

Those skilled in the art will recognize, or be able to ascertain using no more than routine experimentation, many equivalents to the specific embodiments of the invention described herein. Such equivalents are intended to be encompassed by the following embodiments (E).

E1. An isolated nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:2.
E2. An isolated nucleic acid encoding aspartoacyltransferase (ASPA) comprising a nucleic acid sequence comprising or consisting of the sequence of SEQ ID NO:2.
E3. An isolated nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:1.
E4. An isolated nucleic acid encoding aspartoacyltransferase (ASPA) comprising a nucleic acid sequence comprising or consisting of the sequence of SEQ ID NO:1.
E5. An isolated nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:3.
E6. An isolated nucleic acid encoding aspartoacyltransferase (ASPA) comprising a nucleic acid sequence comprising or consisting of the sequence of SEQ ID NO:3.
E7. A modified nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:2.
E8. A modified nucleic acid encoding aspartoacyltransferase (ASPA) comprising a nucleic acid sequence comprising or consisting of the sequence of SEQ ID NO:2.
E9. A modified nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:1.
E10. A modified nucleic acid encoding aspartoacyltransferase (ASPA) comprising a nucleic acid sequence comprising or consisting of the sequence of SEQ ID NO:1.
E11. A modified nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:3.
E12. A modified nucleic acid encoding aspartoacyltransferase (ASPA) comprising a nucleic acid sequence comprising or consisting of the sequence of SEQ ID NO:3.
E13. A recombinant nucleic comprising a modified nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:2.
E14. A recombinant nucleic comprising a modified nucleic acid encoding aspartoacyltransferase (ASPA) comprising or consisting of the nucleic acid sequence of SEQ ID NO:2.
E15. A recombinant nucleic comprising a modified nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:1.
E16. A recombinant nucleic comprising a modified nucleic acid encoding aspartoacyltransferase (ASPA) comprising or consisting of the nucleic acid sequence of SEQ ID NO:1.
E17. A recombinant nucleic comprising a modified nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:3.
E18. A recombinant nucleic comprising a modified nucleic acid encoding aspartoacyltransferase (ASPA) comprising or consisting of the nucleic acid sequence of SEQ ID NO:3.
E19. The recombinant nucleic of any one of E13-E18 further comprising at least one element selected from the group consisting of an enhancer, a promoter, an exon, an intron, and a poly-adenylation (polyA) signal sequence.
E20. The recombinant nucleic of E19 wherein the enhancer comprises a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:6, SEQ ID NO:17 or both.
E21. The recombinant nucleic of any one of E19-E20 wherein the enhancer comprises or consists of the nucleic acid sequence of SEQ ID NO:6, SEQ ID NO:17 or both.
E22. The recombinant nucleic of any one of E19-E21 wherein the promoter is constitutive or regulated.
E23. The recombinant nucleic of any one of E19-E22 wherein the promoter is inducible or repressible.
E24. The recombinant nucleic of any one of E19-E23 wherein the promoter comprises a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:7.
E25. The recombinant nucleic of any one of E19-E24 wherein the promoter comprises or consists of the nucleic acid sequence of SEQ ID NO:7.
E26. The recombinant nucleic of any one of E19-E25 wherein the exon comprises a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:8, SEQ ID NO:18 or both.
E27. The recombinant nucleic of any one of E19-E26 wherein the exon comprises or consists of the nucleic acid sequence of SEQ ID NO:8, SEQ ID NO:18 or both.
E28. The recombinant nucleic of any one of E19-E27 wherein the intron comprises a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:9, SEQ ID NO:10 or both.
E29. The recombinant nucleic of any one of E19-E28 wherein the intron comprises or consists of the nucleic acid sequence of SEQ ID NO:9, SEQ ID NO:10 or both.
E30. The recombinant nucleic of any one of E19-E29 wherein the polyA sequence comprises a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:11.
E31. The recombinant nucleic of any one of E19-E30 wherein the polyA sequence comprises or consists of the nucleic acid sequence of SEQ ID NO:11.
E32. The recombinant nucleic acid of any one of E19-E31 wherein the enhancer is operably linked to the modified nucleic acid.
E33. The recombinant nucleic acid of any one of E19-E32 wherein the promoter is operably linke to the modified nucleic acid.
E34. The recombinant nucleic of any one of E13-E18 further comprising at least one element selected from the group consisting of a cytomegalovirus (CMV) enhancer, a hybrid form of the CBA promoter (CBh promoter), a chicken β-actin (CBA) exon, a CBA intron, a minute virus of mice (MVM) intron and a bovine grown hormone (BGH) polyA.
E35. The recombinant nucleic of any one of E13-E18 further comprising a least one element selected from the group consisting of a CMV enhancer comprising the nucleic acid sequence of SEQ ID NO:6 or SEQ ID NO:17, a CBh promoter comprising the nucleic acid sequence of SEQ ID NO:7, a CBA exon comprising the nucleic acid sequence of SEQ ID NO:8 or SEQ ID NO:18, a CBA intron comprising the nucleic acid sequence of SEQ ID NO:9, an MMV intron comprising the nucleic acid sequence of SEQ ID NO:10 and a BGH polyA comprising the nucleic acid sequence of SEQ ID NO:11.
E36. A vector genome comprising a modified nucleic acid of any one of E7-E12 or a recombinant nucleic acid of any one of E13-E35 wherein the vector genome further comprises at least one AAV ITR repeat sequence comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of SEQ ID NO:5, SEQ ID NO:12 or both.
E37. The vector genome of E36 wherein the at least one AAV ITR repeat sequence comprises or consists of the nucleic acid sequence of SEQ ID NO:5, SEQ ID NO:12, SEQ ID NO:19 or a combination thereof.
E38. The vector genome of E36 or E37 comprising two AAV2 ITR sequences flanking a nucleic acid sequence encoding ASPA and a CBh promoter upstream of the sequence encoding the ASPA.
E39. The vector genome of any one of E36-E38 wherein the ASPA sequence comprises the nucleic acid sequence of SEQ ID NO:2.
E40. The vector genome of any one of E36-E39 wherein the at least one AAV2 ITR sequence comprises the nucleic acid sequence of SEQ ID NO:5, SEQ ID NO:12, SEQ ID NO:19 or a combination thereof.
E41. The vector genome of any one of E36-E40 wherein the CBh promoter comprises the nucleic acid sequence of SEQ ID NO:7.
E42. A vector genome comprising a nucleic acid wherein the nucleic acid comprises from 5′ to 3
a) an AAV2 ITR comprising the nucleic acid sequence of SEQ ID NO:5, SEQ ID NO:12 or SEQ ID NO:19;
b) a CMV enhancer comprising the nucleic acid sequence of SEQ ID NO:6 or SEQ ID NO:17, preferably SEQ ID NO:6;
c) a CBh promoter comprising the nucleic acid sequence of SEQ ID NO:7;
d) a CBA exon comprising the nucleic acid sequence of SEQ ID NO:8, SEQ ID NO:18, preferably SEQ ID NO:18;
e) a CBA intron comprising the nucleic acid sequence of SEQ ID NO:9;
f) an MMV intron comprising the nucleic acid sequence of SEQ ID NO:10;
g) a modified nucleic acid encoding aspartoacyltransferase (ASPA) comprising the nucleic acid sequence of any one of SEQ ID NO:1-3;
h) a BGH polyA comprising the nucleic acid sequence of SEQ ID NO:11; and
i) an AAV2 ITR comprising the nucleic acid sequence of SEQ ID NO:5, SEQ ID NO:12, SEQ ID NO:19.
   E43. A vector genome comprising a nucleic acid wherein the nucleic acid comprises from 5′ to 3′:
a) an AAV ITR comprising the nucleic acid sequence of SEQ ID NO:5, SEQ ID NO:12 or SEQ ID NO:19;
b) an enhancer comprising the nucleic acid sequence of SEQ ID NO:6 or SEQ ID NO:17, preferably SEQ ID NO:6;
c) a promoter comprising the nucleic acid sequence of SEQ ID NO:7;
d) an exon comprising the nucleic acid sequence of SEQ ID NO:8 or SEQ ID NO:18, preferably SEQ ID NO:18;
e) an intron comprising the nucleic acid sequence of SEQ ID NO:9;
f) an intron comprising the nucleic acid sequence of SEQ ID NO:10;
g) a modified nucleic acid encoding aspartoacyltransferase (ASPA) comprising the nucleic acid sequence of any one of SEQ ID NO:1-3;
h) a PolyA comprising the nucleic acid sequence of SEQ ID NO:11; and
i) an AAV terminal repeat comprising the nucleic acid sequence of SEQ ID NO:5, SEQ ID NO:12 or SEQ ID NO:19.
   E44. The vector genome of any one of E36-43, wherein the vector genome is self-complementary.
   E45. A recombinant adeno-associated virus (rAAV) vector comprising the vector genome of any one of E36-E44 and a capsid.
   E46. An rAAV vector comprising a vector genome comprising a nucleic acid sequence about 80%, about 85%, about 90%, about 91%, about 92%, about 93%, about 94%, about 95%, about 96%, about 97%, about 98%, about 99% or 100% identical to the nucleic acid sequence of SEQ ID NO:2.
   E47. The rAAV vector of E46, comprising a capsid selected from the group consisting of a capsid of Olig001, Olig002, Olig003, AAV1, AAV2, AAV3, AAV3A, AAV3B, AAV4, AAV5, AAV6, AAV7, AAV8, AAV9, AAV10, AAV11, AAV12, AAVrh10, AAVrh74, RHM4-1, RHM15-1, RHM15-2, RHM15-3/RHM15-5, RHM15-4, RHM15-6, AAV Hu.26, AAV1.1, AAV2.5, AAV6.1, AAV6.3.1, AAV9.45, AAV2i8, AAV2G9, AAV2i8G9, AAV2-TT, AAV2-TT-S312N, AAV3B-S312N, and AAV-LK03.
   E48. An rAAV vector comprising a vector genome comprising a nucleic acid sequence about 80%, about 85%, about 90%, about 91%, about 92%, about 93%, about 94%, about 95%, about 96%, about 97%, about 98%, about 99% or 100% identical to the nucleic acid sequence of SEQ ID NO:1.
   E49. The rAAV vector of E48, comprising a capsid selected from the group consisting of a capsid of Olig001, Olig002, Olig003, AAV1, AAV2, AAV3, AAV3A, AAV3B, AAV4, AAV5, AAV6, AAV7, AAV8, AAV9, AAV10, AAV11, AAV12, AAVrh10, AAVrh74, RHM4-1, RHM15-1, RHM15-2, RHM15-3/RHM15-5, RHM15-4, RHM15-6, AAV Hu.26, AAV1.1, AAV2.5, AAV6.1, AAV6.3.1, AAV9.45, AAV2i8, AAV2G9, AAV2i8G9, AAV2-TT, AAV2-TT-S312N, AAV3B-S312N, and AAV-LK03.
   E50. An rAAV vector comprising a vector genome comprising a nucleic acid sequence about 80%, about 85%, about 90%, about 91%, about 92%, about 93%, about 94%, about 95%, about 96%, about 97%, about 98%, about 99% or 100% identical to the nucleic acid sequence of SEQ ID NO:3.
   E51. The rAAV vector of E50, comprising a capsid selected from the group consisting of a capsid of Olig001, Olig002, Olig003, AAV1, AAV2, AAV3, AAV3A, AAV3B, AAV4, AAV5, AAV6, AAV7, AAV8, AAV9, AAV10, AAV11, AAV12, AAVrh10, AAVrh74, RHM4-1, RHM15-1, RHM15-2, RHM15-3/RHM15-5, RHM15-4, RHM15-6, AAV Hu.26, AAV1.1, AAV2.5, AAV6.1, AAV6.3.1, AAV9.45, AAV2i8, AAV2G9, AAV2i8G9, AAV2-TT, AAV2-TT-S312N, AAV3B-S312N, and AAV-LK03.
   E52. The rAAV vector of any one of E45-E51 wherein the capsid is selected from an Olig001, an Olig002 and an Olig003 capsid.
   E53. The rAAV vector of any one of E45-E52 wherein the capsid is an Olig001 capsid comprising a viral protein 1 (VP1) and wherein the VP1 comprises an amino acid sequence at least about 70%, 75%, 80%, 85%, 90%, 95%, 98%, 99% or 100% identical to the amino acid sequence of SEQ ID NO:14.
   E54. The rAAV vector of any one of E45-E53 wherein the capsid is an Oligo001 capsid comprising a viral protein 1 (VP1) and wherein the VP1 comprises the amino acid sequence of SEQ ID NO:14.
   E55. The rAAV vector of any one of E45-E52 wherein the capsid is an Olig002 capsid comprising a viral protein 1 (VP1) and wherein the VP1 comprises an amino acid sequence at least about 70%, 75%, 80%, 85%, 90%, 95%, 98%, 99% or 100% identical to the amino acid sequence of SEQ ID NO:15.
   E56. The rAAV vector of any one of E45-E52 and E55 wherein the capsid is an Oligo002 capsid comprising a viral protein 1 (VP1) and wherein the VP1 comprises the amino acid sequence of SEQ ID NO:15.
   E57. The rAAV vector of any one of E45-E52 wherein the capsid is an Olig003 capsid comprising a viral protein 1 (VP1) and wherein the VP1 comprises an amino acid sequence at least about 70%, 75%, 80%, 85%, 90%, 95%, 98%, 99% or 100% identical to the amino acid sequence of SEQ ID NO:16.
   E58. The rAAV vector of any one of E46-E52 and E57 wherein the capsid is an Oligo003 capsid comprising a viral protein 1 (VP1) and wherein the VP1 comprises the amino acid sequence of SEQ ID NO:16.
   E59. The rAAV vector of any one of E45-E58 wherein the vector genome is self-complementary.
   E60. The rAAV vector of any one of E46-E59 wherein the vector genome comprises at least one element selected from the group consisting of at least one AAV inverted terminal repeat (ITR) sequence, an enhancer, a promoter, an exon, an intron, and a poly-adenylation (polyA) signal sequence.
   E61. The rAAV vector of E60 wherein the enhancer comprises a nucleic acid sequence at least 80%, at least 85%, at least 90%, at least 95%, at least 98%, at least 99% or 100% identical to the nucleic acid sequence of SEQ ID NO:6 or SEQ ID NO:17.
   E62. The rAAV vector of E60 or E61 wherein the enhancer comprises or consists of the nucleic acid sequence of SEQ ID NO:6 or SEQ ID NO:17.
   E63. The rAAV vector of any one of E60-E62 wherein the promoter is constitutive or regulated.
   E64. The rAAV vector of any one of E60-E63 wherein the promoter is inducible or repressible.
   E65. The rAAV vector of any one of E60-E64 wherein the promoter comprises a nucleic acid sequence at least 80%, at least 85%, at least 90%, at least 95%, at least 98%, at least 99% or 100% identical to the nucleic acid sequence of SEQ ID NO:7.
   E66. The rAAV vector of any one of E60-E65 wherein the promoter comprises or consists of the nucleic acid sequence of SEQ ID NO:7.
   E67. The rAAV vector of any one of E60-E66 wherein the exon comprises a nucleic acid sequence at least 80%, at least 85%, at least 90%, at least 95%, at least 98%, at least 99% or 100% identical to the nucleic acid sequence of SEQ ID NO:8 or SEQ ID NO:18.
   E68. The rAAV vector of any one of E60-E67 wherein the exon comprises or consists of the nucleic acid sequence of SEQ ID NO:8 or SEQ ID NO:18.
   E69. The rAAV vector of any one of E60-E68 wherein the intron comprises a nucleic acid sequence at least 80%, at least 85%, at least 90%, at least 95%, at least 98%, at least 99% or 100% identical to the nucleic acid sequence of SEQ ID NO:9, SEQ ID NO:10 or both.
   E70. The rAAV vector of any one of E60-E69 wherein the intron comprises or consists of the nucleic acid sequence of SEQ ID NO:9, SEQ ID NO:10 or both.
   E71. The rAAV vector of any one of E60-E70 wherein the polyA sequence comprises a nucleic acid sequence at least 80%, at least 85%, at least 90%, at least 95%, at least 98%, at least 99% or 100% identical to the nucleic acid sequence of SEQ ID NO:11.
   E72. The rAAV vector of any one of E60-E71 wherein the polyA sequence comprises or consists of the nucleic acid sequence of SEQ ID NO:11.
   E73. The rAAV vector of any one of E60-E72 wherein the at least one AAV ITR repeat sequence comprises a nucleic acid sequence at least 80%, at least 85%, at least 90%, at least 95%, at least 98%, at least 99% or 100% identical to the nucleic acid sequence of SEQ ID NO:5, SEQ ID NO:12, SEQ ID NO:19, or a combination thereof.
   E74. The rAAV vector of any one of E60-E73 wherein the at least one AAV ITR repeat sequence comprises or consists of the nucleic acid sequence of SEQ ID NO:5, SEQ ID NO:12, SEQ ID NO:19 or a combination thereof.
   E75. The rAAV vector of any one of E46-E59 wherein the vector genome further comprises at least one element selected from the group consisting of at least one AAV2 ITR sequence, a CMV enhancer, a CBh promoter, a CBA exon 1, a CBA intron 1, an MVM intron and a BGH polyA.
   E76. The rAAV vector of any one E46-E59 wherein the vector genome further comprises a least one element selected from the group consisting of at least one AAV2 ITR sequence comprising the nucleic acid sequence of SEQ ID NO:5, SEQ ID NO:12, SEQ ID NO:19, or a combination thereof, a CMV enhancer comprising the nucleic acid sequence of SEQ ID NO:6 or SEQ ID NO:17, a CBh promoter comprising the nucleic acid sequence of SEQ ID NO:7, a CBA exon 1 comprising the nucleic acid sequence of SEQ ID NO:8 or SEQ ID NO:18, a CBA intron 1 comprising the nucleic acid sequence of SEQ ID NO:9, an MMV intron comprising the nucleic acid sequence of SEQ ID NO:10 and a BGH polyA comprising the nucleic acid sequence of SEQ ID NO:11.
   E77. The rAAV vector of any one of E46-E59 wherein the vector genome comprises two AAV2 ITR sequences flanking a sequence encoding ASPA and a CBh promoter upstream of the sequence encoding the ASPA.
   E78. The rAAV vector of E77 wherein the ASPA sequence comprises the nucleic acid sequence of SEQ ID NO:2.
   E79. The rAAV vector of E77 or E78, wherein the AAV ITR sequences comprise the nucleic acid sequence of SEQ ID NO:5, SEQ ID NO:12, SEQ ID NO:19 or a combination thereof.
   E80. The rAAV vector of any one of E77-E79 wherein the CBh promoter comprises the nucleic acid sequence of SEQ ID NO:7.
   E81. An rAAV vector comprising a vector genome comprising from 5′ to 3′:
a) an AAV2 ITR comprising the nucleic acid sequence of SEQ ID NO:5, SEQ ID NO:12, SEQ ID NO:19 or a combination thereof;
b) a CMV enhancer comprising the nucleic acid sequence of SEQ ID NO:6 or SEQ ID NO:16;
c) a CBh promoter comprising the nucleic acid sequence of SEQ ID NO:7;
d) a CBA exon 1 comprising the nucleic acid sequence of SEQ ID NO:8 or SEQ ID NO:18;
e) a CBA intron 1 comprising the nucleic acid sequence of SEQ ID NO:9;
f) an MMV intron comprising the nucleic acid sequence of SEQ ID NO:10;
g) a modified nucleic acid encoding aspartoacyltransferase (ASPA) comprising the nucleic acid sequence of any one of SEQ ID NO:1-3;
h) a BGH polyA comprising the nucleic acid sequence of SEQ ID NO:11; and
i) an AAV2 ITR comprising the nucleic acid sequence of SEQ ID NO:5, SEQ ID NO:12, SEQ ID NO; 19 or a combination thereof.
E82. An rAAV vector comprising a vector genome comprising from 5′ to 3′:
   a) an AAV ITR comprising the nucleic acid sequence of SEQ ID NO:5, SEQ ID NO:12, SEQ ID NO:19 or a combination thereof;
   b) an enhancer comprising the nucleic acid sequence of SEQ ID NO:6 or SEQ ID NO:17;
   c) a promoter comprising the nucleic acid sequence of SEQ ID NO:7;
   d) an exon comprising the nucleic acid sequence of SEQ ID NO:8 or SEQ ID NO:18;
   e) an intron comprising the nucleic acid sequence of SEQ ID NO:9;
   f) an intron comprising the nucleic acid sequence of SEQ ID NO:10;
   g) a modified nucleic acid encoding aspartoacyltransferase (ASPA) comprising the nucleic acid sequence of any one of SEQ ID NO:1-3;
   h) a PolyA comprising the nucleic acid sequence of SEQ ID NO:11; and
   i) an AAV terminal repeat comprising the nucleic acid sequence of SEQ ID NO:5, SEQ ID NO:12, SEQ ID NO:19 or a combination thereof.
      E83. The rAAV vector of E81 or E82 wherein the vector genome is self-complementary.
      E84. The rAAV vector of any one of E81-E83 wherein the vector comprises an Olig001 capsid comprising a VP1 protein wherein the VP1 comprises an amino acid sequence at least 80%, at least 85%, at least 90%, at least 95%, at least 98%, at least 99% or 100% identical to the amino acid sequence of SEQ ID NO:14.
      E85. The rAAV vector of any one of E81-E83 wherein the vector comprises an Olig002 capsid comprising a VP1 protein wherein the VP1 comprises an amino acid sequence at least 80%, at least 85%, at least 90%, at least 95%, at least 98%, at least 99% or 100% identical to the amino acid sequence of SEQ ID NO:15.
      E86. The rAAV vector of any one of E81-E83 wherein the vector comprises an Olig003 capsid comprising a VP1 protein wherein the VP1 comprises an amino acid sequence at least 80%, at least 85%, at least 90%, at least 95%, at least 98%, at least 99% or 100% identical to the amino acid sequence of SEQ ID NO:16.
      E87. An rAAV vector comprising i) an Olig001 capsid comprising a VP1 protein wherein the VP1 comprises the amino acid sequence of SEQ ID NO:14 and ii) a self-complementary vector genome comprising from 5′ to 3′:
   a) an AAV2 ITR comprising the nucleic acid sequence of SEQ ID NO:5, SEQ ID NO:12, SEQ ID NO:19 or a combination thereof;
   b) a CMV enhancer comprising the nucleic acid sequence of SEQ ID NO:6 or SEQ ID NO:17;
   c) a CBh promoter comprising the nucleic acid sequence of SEQ ID NO:7;
   d) a CBA exon 1 comprising the nucleic acid sequence of SEQ ID NO:8 or SEQ ID NO:18;
   e) a CBA intron 1 comprising the nucleic acid sequence of SEQ ID NO:9;
   f) an MMV intron comprising the nucleic acid sequence of SEQ ID NO:10;
   g) a modified nucleic acid encoding aspartoacyltransferase (ASPA) comprising the nucleic acid sequence of any one of SEQ ID NO:1-3;
   h) a BGH polyA comprising the nucleic acid sequence of SEQ ID NO:11; and
   i) an AAV2 ITR comprising the nucleic acid sequence of SEQ ID NO:5 or SEQ ID NO:12.
      E88. An rAAV vector comprising i) an Olig001 capsid comprising a VP1 protein wherein the VP1 comprises the amino acid sequence of SEQ ID NO:14 and ii) a self-complementary vector genome comprising from 5′ to 3′:
   a) an AAV ITR comprising the nucleic acid sequence of SEQ ID NO:5, SEQ ID NO:12, SEQ ID NO:19 or a combination thereof;
   b) an enhancer comprising the nucleic acid sequence of SEQ ID NO:6 or SEQ ID NO:17;
   c) a promoter comprising the nucleic acid sequence of SEQ ID NO:7;
   d) an exon comprising the nucleic acid sequence of SEQ ID NO:8 or SEQ ID NO:18;
   e) an intron comprising the nucleic acid sequence of SEQ ID NO:9;
   f) an intron comprising the nucleic acid sequence of SEQ ID NO:10;
   g) a modified nucleic acid encoding aspartoacyltransferase (ASPA) comprising the nucleic acid sequence of any one of SEQ ID NO:1-3;
   h) a PolyA comprising the nucleic acid sequence of SEQ ID NO:11; and
   i) an AAV ITR comprising the nucleic acid sequence of SEQ ID NO:5, SEQ ID NO:12, SEQ ID NO:19 or a combination thereof.
      E89. The rAAV vector of any one of E45-E88 wherein the vector, when introduced into a cell, decreases the level of NAA in the cell.
      E90. The rAAV vector of E89 wherein the cell is a brain cell.
      E91. The rAAV vector of E89 or E90 where the cell is an oligodendrocyte.
      E92. The rAAV vector of any one of E45-E91 wherein administration of the vector to a subject with an ASPA gene mutation increases balance, grip strength and/or motor coordination in the subject as compared to balance, grip strength and/or motor coordination in the subject before administration of the vector.
      E93. The rAAV vector of any one of E45-E92 wherein administration of the vector to a subject with an ASPA gene mutation increases generalized motor function in the subject as compared to generalized motor function in the subject before administration of the vector.
      E94. The rAAV vector of any one of E45-E93 wherein administration of the vector to a subject with an ASPA gene mutation decreases NAA levels in the subject as compared to NAA levels in the subject before administration of the vector.
      E95. The rAAV vector of any one of E45-E94 wherein administration of the vector to a subject with an ASPA gene mutation decreases vacuole volume fraction in the thalamus of the subject as compared to vacuole volume fraction in the thalamus of the subject before administration of the vector.
      E96. The rAAV vector of any one of E45-E95 wherein administration of the vector to a subject with an ASPA gene mutation decreases vacuole volume fraction in the cerebellar white matter/pons of the subject as compared to vacuole volume fraction in the cerebellar white matter/pons of the subject before administration of the vector.
      E97. The rAAV vector of any one of E45-E96 wherein administration of the vector to a subject with an ASPA gene mutation increases the number of oligodendrocytes in the thalamus of the subject as compared to the number of oligodendrocytes in the thalamus of the subject before administration of the vector.
      E98. The rAAV vector of any one of E45-E97 wherein administration of the vector to a subject with an ASPA gene mutation increases the number of oligodendrocytes in the brain cortex of the subject as compared to the number of oligodendrocytes in the brain cortex of the subject before administration of the vector.
      E99. The rAAV vector of any one of E45-E98 wherein administration of the vector to a subject with an ASPA gene mutation increases the number of neurons in the thalamus of the subject as compared to the number of neurons in the thalamus of the subject before administration of the vector.
      E100. The rAAV vector of any one of E45-E99 wherein administration of the vector to a subject with an ASPA gene mutation increases the number of neurons in the brain cortex of the subject as compared to the number of neurons in the brain cortex of the subject before administration of the vector.
      E101. The rAAV vector of any one of E45-E100 wherein administration of the vector to a subject with an ASPA gene mutation increases cortical myelination in the subject as compared to cortical myelination in the subject before administration of the vector.
      E102. The rAAV vector of any one of E92-E101 wherein the subject is a human patient.
      E103. The rAAV vector of any one of E92-E102 wherein the subject is a human patient with Canavan disease, or at-risk of developing Canavan disease.
      E104. The rAAV vector of any one of E92-E103 wherein the subject has at least one ASPA gene mutation.
      E105. A pharmaceutical composition comprising the modified nucleic acid of any one of E7-E12, the recombinant nucleic acid of any one of E13-E35, the vector genome of any one of E36-E44 or the rAAV vector of any one of E45-E104.
      E106. A pharmaceutical composition comprising the modified nucleic acid of any one of E7-E12, the recombinant nucleic acid of any one of E13-E35, the vector genome of any one of E36-E44 or the rAAV vector of any one of E45-E104 and a pharmaceutically acceptable carrier.
      E107. A method of treating and/or preventing a disease, disorder or condition associated with deficiency or dysfunction of ASPA, the method comprising administering a therapeutically effective amount of the modified nucleic acid of any one of E7-E12, the recombinant nucleic acid of any one of E13-E35, the vector genome of any one of E36-E44, the rAAV vector of any one of E45-E104 or the pharmaceutical composition of E105 or E106 to a subject in need of treatment.
      E108. The method of E107 wherein the disease, disorder or condition associated with deficiency or dysfunction of ASPA is Canavan disease.
      E109. The method of E107 or E108 wherein the modified nucleic acid, recombinant nucleic acid, vector genome, rAAV vector or pharmaceutical composition is administered directly to the brain of a subject in need of treatment.
      E110. The method of any one of E107-E109 wherein the modified nucleic acid, recombinant nucleic acid, vector genome, rAAV vector or pharmaceutical composition is administered directly to the central nervous system of a subject in need of treatment.
      E111. The method of any one of E107-E110 wherein the modified nucleic acid, recombinant nucleic acid, vector genome, rAAV vector or pharmaceutical composition is administered to at least one region of the central nervous system selected from the group consisting of the brain parenchyma, spinal canal, subarachnoid space, a ventricle of the brain, cisterna magna and any combination thereof.
      E112. The method of any one of E107-E111 wherein the modified nucleic acid, recombinant nucleic acid vector genome, rAAV vector or pharmaceutical composition is administered by at least one method selected from the group consisting of intraparenchymal administration, intrathecal administration, intracerebroventricular administration, intracisternal magna administration and any combination thereof.
      E113. The method of any one of E107-E112 wherein the subject is a human patient.
      E114. The method of any one of E107-E113 wherein the subject is a human patient with Canavan disease or at-risk for developing Canavan disease.
      E115. The method of any one of E107-E114 wherein the subject has at least one mutation in the ASPA gene.
      E116. A method of treating or preventing Canavan disease, the method comprising the steps of: i) assessing whether a subject comprises at least one ASPA gene mutation and ii) administering to the subject a therapeutically effective amount of the modified nucleic acid of any one of E7-E12, the recombinant nucleic acid of any one of E13-E35, the vector genome of any one of E36-E44, the rAAV vector of any one of E45-E104 or the pharmaceutical composition of E105 or E106, thereby treating or preventing Canavan disease in the subject.
      E117. The method of EE116, wherein the subject is diagnosed with Canavan disease or diagnosed as at-risk for developing Canavan disease.
      E118. A method of treating or preventing a disease associated with ASPA deficiency in a subject in need thereof, comprising administering to the subject a therapeutically effective amount of a modified nucleic acid encoding ASPA wherein the modified nucleic acid encoding ASPA has been codon-optimized.
      E119. The method of E118 wherein the modified nucleic acid encoding ASPA comprises the nucleic acid sequence of SEQ ID NO:2.
      E120. The method of E118 or E119 wherein the modified nucleic acid encoding ASPA encodes an ASPA protein having the amino acid sequence of SEQ ID NO:4.
      E121. The method of any one of E118-E120 wherein the modified nucleic acid encoding ASPA is expressed in a target cell and wherein the target cell is an oligodendrocyte.
      E122. The method of any one of E118-E121 wherein the modified nucleic acid encoding ASPA is delivered in a vector to the target cell.
      E123. The method of E122, wherein the vector is a viral vector or a non-viral vector.
      E124. The method of any one of E118-E123 wherein the vector is administered to the subject by systemic injection, by direct intracranial injection or by direct spinal canal injection.
      E125. A host cell comprising the modified nucleic acid of any one of the modified nucleic acid of any one of E7-E12, the recombinant nucleic acid of any one of E13-E35, the vector genome of any one of E36-E44 or the rAAV vector of any one of E45-E104.
      E126. The host cell of E125, wherein the cell is selected from the group consisting of VERO, W138, MRCS, A549, HEK293, B-50 or any other HeLa cell, HepG2, Saos-2, HuH7, and HT1080.
      E127. The host cell of E125-E126 wherein the cell is a HEK293 cell adapted to growth in suspension culture.
      E128. The host cell of any one of E125-E127 wherein the cell is a HEK293 cell having American Type Culture Collection (ATCC) No. PTA 13274.
      E129. The host cell of any one of E125-E128 wherein the cell comprises at least one nucleic acid encoding at least one protein selected from the group consisting of an AAV Rep protein, an AAV capsid (Cap) protein, a adenovirus early region 1A (Ela) protein, a E1b protein, an E2a protein, an E4 protein and a viral associated (VA) RNA.
      E130. A kit for the treatment of Canavan disease (CD), comprising a therapeutically effective amount of i) an rAAV vector of any one of E45-E104 or ii) a pharmaceutical composition of E105 or E106.
      E131. The kit of E130 wherein the kit further comprises a label or insert including instructions for using one or more of the kit components.
      E132. A modified nucleic acid of any one of E7-E12, a recombinant nucleic acid of any one of E13-E35, a vector genome of any one of E36-E44, an rAAV vector of any one of E45-E104 or a pharmaceutical composition of E105 or E106 for use in treating or preventing a disease, disorder or condition associated with deficiency or dysfunction of ASPA.
      E133. The modified nucleic acid, the recombinant nucleic acid, the vector genome, the rAAV vector, or the pharmaceutical composition for use of E132, wherein the disease, disorder or condition is Canavan disease.
      E134. Use of a modified nucleic acid of any one of E7-E12, a recombinant nucleic acid of any one of E13-E35, a vector genome of any one of E36-E44, an rAAV vector of any one of E45-E104 or a pharmaceutical composition of E105 or E106 in the manufacture of a medicament for treating and/or preventing a disease, disorder of condition associated with deficiency or dysfunction of ASPA.
      E135. The use of E134 wherein the disease, disorder or condition is Canavan disease.
      E136. A method of determining biodistribution of a transgene delivered by an rAAV vector comprising an Olig001 capsid to the brain of a subject wherein a protein encoded by the transgene is expressed, the method comprising
   a) administration of the rAAV vector to the subject;
   b) fixation of the brain tissue;
   c) electrophoretic clearing of the brain;
   d) 3D microscopic imaging of a brain tissue section;
   e) detection of the protein;
   f) optionally, quantification of the amount of protein present in the brain tissue.
      E137. The method of E136 wherein the administration is by intracrebroventricular (ICV) injection, intraparenchymal (IP) injection, intrathecal (IT) administration, intracisternal magna (ICM) injection or a combination thereof.
      E138. The method of E136 or 137 wherein the brain tissue is fixed using, for example, paraformaldehyde or formalin.
      E139. The method of any one of E136-E138 wherein the quantification includes volumetric rendering.
      E140. The method of any one of E136-E139 wherein the transgene encodes a green fluorescent protein (GFP).
      E141. The method of any one of E136-E140 wherein the level of transgene expression detected in the tissue correlates with rAAV vector transduction efficiency.
      E142. The method of any one of E136-E141, further comprising (g) the step of evaluation of cell-type vector tropism by assessment of cell morphology and spatial location determination of GFP expression.
      E143. A modified nucleic acid encoding aspartoacyltransferase (ASPA) comprising a nucleic acid sequence at least about 80%, about 85%, about 90%, about 91%, about, 92%, about 93%, about 94%, about 95%, about 96%, about 97%, about 98%, about 99% or 100% identical to the nucleic acid sequence of any one of SEQ ID NO:1-3 and a promoter.
      E144. A modified nucleic acid encoding aspartoacyltransferase (ASPA) comprising a nucleic acid comprising or consisting of the sequence of SEQ ID NO:2 and a promoter.
      E145. A nucleic acid comprising a nucleic acid sequence encoding a promoter, and further comprising a modified nucleic acid sequence encoding ASPA, wherein the modified nucleic acid sequence comprises or consists of the sequence of SEQ ID NO:2.
      E146. An isolated nucleic acid comprising a nucleic acid sequence specifying a promoter and further comprising a nucleic acid sequence comprising or consisting of the nucleic acid sequence of SEQ ID NO:2.
      E147. The pharmaceutical composition of E105 further comprising 350 mM NaCl and 5% D-sorbitol in PBS.
      E148. The pharmaceutical composition of E106 wherein the pharmaceutically acceptable carrier comprises 350 mM NaCl and 5% D-sorbitol in PBS.

Other features and advantages of the invention will be apparent from the following detailed description, drawings, exemplary embodiments and claims.

### BRIEF DESCRIPTION OF THE DRAWINGS

FIG. 1 depicts an exemplary dose response reduction in NAA as determined using HPLC in cells transfected with 1.0 μg plasmid expressing NAA synthase (Nat8L) and co-transfected with 1.0 μg, 0.5 μg, 0.2 μg or 0.1 μg of a plasmid comprising the wild type human ASPA sequence (SEQ ID NO:3) or a modified, e.g., codon-optimized ASPA sequence original (version 1) (SEQ ID NO:1) or codon-optimized ASPA sequence new (version 2) (SEQ ID NO:2).

FIG. 2 depicts exemplary sampling of GFP positive cells transduced by rAAV vector administered via the intraparenchymal (IP) route of administration (ROA). GFP-positive soma (arrow) were scored in each region of interest to generate estimates of number (N) of transduced cells.

FIG. 3 depicts an exemplary number of GFP-positive cells (N) in the cortex, subcortical white matter of the corpus callosum and external capsule, striatum and cerebellum of 6 week old nur7 mice following intraparenchymal (IP) administration of AAV/Olig001-GFP and a representative image of native GFP fluorescence in a sagittal section of a brain from a mouse to which 1×10¹¹ AAV/Olig001-GFP vector genomes were administered via the IP ROA showing concentrated GFP expression adjacent to injection sites. Estimates of N were generated in 144 sections using the optical fractionator (k=4). Mean+/−sem for each group presented (n=5 animals). Significant differences in numbers of GFP-positive cells between dose cohorts within each region of interest are denoted by asterisks.

FIG. 4 depicts an exemplary number of GFP-positive cells (N) in the cortex, subcortical white matter, striatum and cerebellum of 6 week old nur7 mice following intrathecal (IT) administration of AAV/Olig001-GFP and a representative image of native GFP fluorescence in a sagittal section of a brain from a mouse to which 1×10¹¹ AAV/Olig001-GFP vector genomes were administered via the intrathecal (IT) ROA showing diffuse cortical marker expression demonstrating transduction by the vector and modest white matter tract cell expression also demonstrating transduction of cells in that region. Mean+/−sem for each group presented (n=5 animals). Significant differences in numbers of GFP-positive cells between dose cohorts within each region of interest are denoted by asterisks.

FIG. 5 depicts an exemplary number of GFP-positive cells (N) in the cortex, subcortical white matter, striatum and cerebellum in 6 week old nur7 mice following intracerebroventricular (ICV) administration of AAV/Olig001-GFP and a representative image of native GFP fluorescence in a sagittal section of a brain of a mouse to which 1×10¹¹ AAV/Olig001-GFP vector genomes were administered via the ICV ROA showing intense white matter tract GFP expression demonstrating transduction by the vector of cells in that region. Mean+/−sem for each group presented (n=5 animals). Significant differences in numbers of GFP-positive cells between dose cohorts within each region of interest are denoted by asterisks.

FIG. 6 depicts an exemplary number of GFP-positive cells (N) in the cortex, subcortical white matter, striatum and cerebellum in 6 week old nur7 mice following intracisternal magna (ICM) administration of AAV/Olig001-GFP and a representative image of native GFP fluorescence in a sagittal section of a brain from a mouse to which 1×10¹¹ AAV/Olig001-GFP vector genomes were administered via the ICM ROA showing modest white matter tract GFP marker expression demonstrating transduction of cells in that region. Mean+/−sem for each group presented (n=5 animals). Significant differences in numbers of GFP-positive cells between dose cohorts within each region of interest are denoted by asterisks.

FIG. 7 depicts direct comparison of exemplary AAV/Olig001-GFP transduction efficiency in four regions of interest: cortex, subcortical white matter, striatum and cerebellum for a 1×10¹¹ vg dose administered to each animal by 4 different routes of administration (IP, IT, ICV and ICM) and representative images of native GFP fluorescence in sections lateral to injection sites in intraparenchymal and intracerebroventricular brains. Both cortical and subcortical white matter tract transgene-positive cells were more numerous in lateral sections in ICV brains. For each group, n=5 animals, with mean+/−sem. Significant differences in numbers of GFP-positive cells between individual region of interest are denoted by asterisks (*p≤0.05, **p≤0.01 and ***p≤0.001).

FIG. 8 depicts exemplary oligotropism of AAV/Olig001-GFP in the cortex of 6 week old nur7 mice following intraparenchymal (IP), intrathecal (IT), intracerebroventricular (ICV) and intracisternal magna (ICM) vector administration. Cortical sections were analyzed by IHC using Olig2 and NeuN antibodies. For each group, n=5 animals, with mean percentage of co-labeling with each indicated antigen+/−sem. Asterisk indicates a significant difference between groups.

FIG. 9 depicts exemplary oligotropism of AAV/Olig001-GFP in the subcortical white matter of 6 week old nur7 mice following intraparenchymal (IP), intrathecal (IT), intracerebroventricular (ICV) and intracisternal magna (ICM) vector administration. Sections of subcortical white matter were analyzed by IHC using Olig2 and NeuN antibodies. For each group, n=5 animals, with mean percentage of co-labeling with each indicated antigen+/−sem.

FIG. 10 depicts exemplary oligotropism of AAV/Olig001-GFP in the striatum matter of 6 week old nur7 mice following intraparenchymal (IP), intrathecal (IT), intracerebroventricular (ICV) and intracisternal magna (ICM) vector administration where marker detection demonstrates transduction of cells by the vector. Sections of striatum matter were analyzed by IHC using Olig2 and NeuN antibodies. For each group, n=5 animals, with mean percentage of co-labeling with each indicated antigen+/−sem.

FIG. 11 depicts exemplary oligotropism of AAV/Olig001-GFP in the cerebellum of 6 week old nur7 mice following intraparenchymal (IP), intrathecal (IT), intracerebroventricular (ICV) and intracisternal magna (ICM) vector administration where marker detection demonstrates transduction by the vector. Cerebellar sections were analyzed by IHC using Olig2 and NeuN antibodies. For each group, n=5 animals, with mean percentage of co-labeling with each indicated antigen+/−sem.

FIG. 12 depicts exemplary efficiency of AAV/Olig001-GFP transduction in the cortex and subcortical white matter of age-matched wild type (WT) and nur7 mouse brains 2 weeks-post ICV administration of 1×10¹¹ vector genomes and a representative image of native GFP fluorescence in a wild type brain following administration of AAV/Olig001-GFP, showing relatively restricted expression, and thereby demonstrating transduction by the vector, particularly in subcortical white matter. For each group, n=5 animals, mean GFP-positive cell numbers per group+/−sem, *p≤0.05, **p≤0.01.

FIG. 13 depicts an expression plasmid encoding a codon-optimized ASPA coding sequence and regulatory elements.

FIG. 14 depicts exemplary rotarod latency to fall over the course of in-life study period for AAV/Olig001-ASPA treated (at three dose levels), wild type and nur7 sham treated mice. The data are presented as mean+/−sem with n=12 animals per group.

FIG. 15 depicts exemplary open field activity over the course of in-life study period for wild type (WT) mice, AAV/Olig001-ASPA treated (at three dose levels), and sham treated nur7 mice. The data are presented as mean+/−sem with n=12 animals per group.

FIG. 16 depicts exemplary NAA content of wild type (WT), nur7 sham treated and AAV/Olig001-ASPA treated (a three dose levels) mouse brains. Data are expressed as mean+/−sem. NAA is expressed as mmoles per gram of wet tissue weight (n=6 animals per group). The dose of AAV/Olig001-ASPA is indicated on the x-axis.

FIG. 17 depicts exemplary mean vector genome copy number per mg of brain tissue (vg/mg) for nur7 mice treated at 3 different dose levels with AAV/Olig001-ASPA assessed at 22 weeks of age. Mean vg/mg values are presented as +/−sem (n=6 animals per dose cohort).

FIG. 18 depicts representative H&E stained brain sections from nur7 sham treated, AAV/Olig001-ASPA treated nur7 and wild type mice demonstrating areas of vacuolation.

FIG. 19 depicts exemplary vacuole volume fraction as a percentage of region of interest (ROI) of the thalamus and cerebral white matter/pons of brains from 22 week old sham treated and AAV/Olig001-ASPA treated nur7 mice. Asterisks indicate a significant difference between groups.

FIG. 20 depicts representative images of sham treated and AAV/Olig001-ASPA treated (2.5×10¹¹ vg dose) nur7 mouse thalamus and cortex stained for Olig2 demonstrating oligodendrocytes.

FIG. 21 depicts exemplary counts of Olig2 positive cells in the thalamus and cortex of 22 week old wild type, sham treated and AAV/Olig001-ASPA treated nur7 mice. Data expressed as mean Olig2 positive cells+/−sem (n=6 animals per group). Asterisks indicate a significant difference between groups.

FIG. 22 depicts representative images of sham treated, and AAV/Olig001-ASPA treated (2.5×10¹¹ vg dose) nur7 mouse thalamus and cortex stained for NeuN.

FIG. 23 depicts exemplary counts of NeuN positive cells in the thalamus and cortex of 22 week old wild type, sham treated and AAV/Olig001-ASPA treated nur7 mice. Data expressed as mean NeuN positive cells+/−sem (n=6 animals per group). Asterisks indicate a significant difference between groups.

FIG. 24 depicts representative images of sham treated and AAV/Olig001-ASPA treated (2.5×10¹¹ vg dose) nur7 mouse cortex stained for myelin basic protein (MBP).

FIG. 25 depicts exemplary myelin basic protein positive fiber length density (MBP-LD) (μm/mm³) in wild type, sham treated and AAV/001-ASPA treated nur7 mouse cortex. Data expressed as mean MBP-LD+/−sem (n=6 animals per group). Asterisks indicate a significant difference between groups.

FIG. 26 depicts exemplary brain images from an ICV injected mouse from an initial fixed, pre-cleared sample, a post-tissue cleared sample, a 3D GFP fluorescence image, a hemibrain volumetric segmentation analysis and an intensity heatmap (left to right).

FIG. 27 depicts intensity heatmaps from all four ICV injected hemibrains. Full hemibrain volume is calculated and represented as gray areas. Calculated “low” GFP intensity is indicated in the gray areas; “high” GFP intensity is indicated in the white areas.

FIG. 28 depicts 3D lightsheet GFP fluorescence microscopy images from cleared brains of animals administered AAV/Oligo001-GFP via ICV versus IP routes of administration.

FIG. 29A depicts representative high magnification images showing scoring of GFP-positive cells co-labelled with Olig2 or NeuN. Total GFP cells were scored in each field of view, and the percentage of Olig2 and NeuN co-labelling scored within the same field.

FIG. 29B depicts representative images of co-labelling of GFP with Olig2 in SCWM tract cells in the brain of an animal given AAV/Olig001-GFP via the ICV ROA and demonstrating near 100% oligotropism and a near complete absence of neurotropism.

FIG. 29C depicts a representative image of cerebellar GFP transgene expression in large purkinje neurons, with sparse Olig2 co-labelling in white matter (arrow).

FIG. 29D depicts a representative image of GFP co-labeling with Olig2 in the striatum of an ICV ROA brain, showing contrast with cerebellar tropism.

FIG. 29E depicts representative images of white matter tracts in 8-week nur7 and age-matched wild type naïve brains after processing for BrdU labeling and Olig2.

FIG. 29F depicts exemplary counts of BrdU cells in 2-week and 8-week wild type and nur7 white matter tracts. Mean BrdU-positive cells per group+/−sem presented. For each group (genotype at each age), n=6.

FIG. 29G depicts representative images of BrdU/GFP co-labelled cells in subcortical white matter of a nur7 brain treated with AAV/Olig001-GFP via the ICV ROA.

FIGS. 30A, 30B, and 30C depict biodistribution volumetric analysis. (A) Volumes of tissues imaged across both ICV and IP. (B) Average and median GFP fluorescence intensities across the two ROAs. (C) Fractions of volumetric GFP positivity representing low and high intensities across ROAs.

FIGS. 31A, 31B, and 31C depict CLARITY and SWITCH workflow for pharmacodynamics effect evaluation. (A) Tissue clearing and labeling approach. From left to right: an intact mouse brain, a central 2-mm section of right hemibrain prior to clearing, the same tissue after 1 day of passive clearing and after 3 days of passive clearing, and a 3D image displaying fluorescence signal from previously labeled proteins (green: nuclei, red: myelin basic protein (MBP). (B) Representative 2-mm sections of Nur7, WT and Olig1-ASPA treated tissues. Red arrowhead in each image indicates the thalamic region. (C) Tissue transparency after one day of passive clearing.

FIGS. 32A, 32B, and 32C depict 2D region-based cell counting of tissues. (A) Extracted 2D single slices of 3D images from all three groups with similar anatomical orientation. Red boxes mark areas in the thalamic and cortical region where cell counting was performed. (B) Image data enlarged from the red boxes in (A), and respective cell segmentation. (C) Average nuclei density (counts normalized by segmentation area).

FIGS. 33A, 33B, 33C, 33D, 33E, 33F, 33G, 33H, and 33I depict 3D volumetric analysis of pharmacodynamic treatment effect. (A) A full 3D volume of a 2-mm tissue slice is determined. (B) The average fluorescence intensity calculated within the 3D volume. (C) MBP characterization via a more restrictive threshold set at fluorescence value of over 2000 (left panel) or a more inclusive threshold at 1000 (left panel). (D) In both cases, MBP deficit in Nur7 can be observed. An effect of the Olig1-ASPA group can be seen in the lower threshold, where the overall value approaches WT levels. (E) Region-based 3D analyses in the thalamic region where a manual segmentation of a portion of the region is shown in yellow. (F) Average fluorescence within this region for both nuclei (SYTO) and myelin (MBP) markers. (G) Region-based analysis on a portion of the cortex where the manual segmentation is shown in yellow. (H) Average fluorescence within this cortical region for both nuclei (SYTO) and myelin (MBP) markers. (I) 3D cell concentration (nuclei per 100 um²).

### DETAILED DESCRIPTION OF THE INVENTION

### Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The following terms have the meanings given:

As used herein, the term “about,” or “approximately” refers to a measurable value such as an amount of the biological activity, length of a polynucleotide or polypeptide sequence, content of G and C nucleotides, codon adaptation index, number of CpG dinucleotides, dose, time, temperature, and the like, and is meant to encompass variations of 25%, 20%, 19%, 18%, 17%, 16%, 15%, 14%, 13%, 12%, 11%, 10%, 9%, 8%, 7%, 6%, 5%, 4%, 3%, 2% 1%, 0.5% or even 0.1%, in either direction (greater than or less than) of the specified amount unless otherwise stated, otherwise evident from the context, or except where such number would exceed 100% of a possible value.

As used herein, the term “and/or” refers to and encompasses any and all possible combinations of one or more of the associated listed items, as well as the lack of combinations when interpreted in the alternative (“or”).

As used herein, the terms “adeno-associated virus” and/or “AAV” refer to a parvovirus with a linear single-stranded DNA genome and variants thereof. The term covers all subtypes and both naturally occurring and recombinant forms, except where required otherwise. The wild-type genome comprises 4681 bases (Berns and Bohenzky (1987) Advances in Virus Research 32:243-307) and includes terminal repeat sequences (e.g., inverted terminal repeats (ITRs)) at each end which function in cis as origins of DNA replication and as packaging signals for the virus. The genome includes two large open reading frames, known as AAV replication (“AAV rep” or “rep”) and capsid (“AAV cap” or “cap”) genes, respectively. AAV rep and cap may also be referred to herein as AAV “packaging genes.” These genes code for the viral proteins involved in replication and packaging of the viral genome.

In wild type AAV virus, three capsid genes VP1, VP2 and VP3 overlap each other within a single open reading frame and alternative splicing leads to production of VPI, VP2 and VP3. (Grieger and Samulski (2005) J. Virol. 79(15):9933-9944.) A single P40 promoter allows all three capsid proteins to be expressed at a ratio of about 1:1:10 for VP1, VP2, VP3, respectively, which complements AAV capsid production. More specifically, VP1 is the full-length protein, with VP2 and VP3 being increasingly shortened due to increasing truncation of the N-terminus. A well-known example is the capsid of AAV9 as described in U.S. Pat. No. 7,906,111, wherein VP1 comprises amino acid residues 1 to 736 of SEQ ID NO:123, VP2 comprises amino acid residues 138 to 736 of SEQ ID NO:123, and VP3 comprises amino acid residues 203 to 736 of SEQ ID NO:123. As uses herein, the term “AAV Cap” or “cap” refers to AAV capsid proteins VP1, VP2 and/or VP3, and variants and analogs thereof.

At least four viral proteins are synthesized from the AAV rep gene, Rep 78, Rep 68, Rep 52 and Rep 40, and are named according to their apparent molecular weights. As used herein, “AAV rep” or “rep” means AAV replication proteins Rep 78, Rep 68, Rep 52 and/or Rep 40, as well as variants and analogs thereof. As used herein, rep and cap refer to both wild type and recombinant (e.g., modified chimeric, and the like) rep and cap genes as well as the polypeptides they encode. In some embodiments, a nucleic acid encoding a rep will comprise nucleotides from more than one AAV serotype. For instance, a nucleic acid encoding a rep may comprise nucleotides from an AAV2 serotype and nucleotides from an AA3 serotype (Rabinowitz et al. (2002) J. Virology 76(2):791-801).

As used herein the terms “recombinant adeno-associated virus vector,” “rAAV” and/or “rAAV vector” refer to an AAV comprising a vector genome wherein a polynucleotide sequence not of, or not entirely of, AAV origin (e.g., a polynucleotide heterologous to AAV), and wherein the rep and/or cap genes of the wild type AAV virus genome have been removed from the virus genome. Where the rep and/or cap genes of the canonical AAV have been removed or are not present (and where the flanking ITRs are typically derived from ITRs from a different serotype, such as, but not limited to AAV2 ITRs where the capsid is not AAV2), the nucleic acid within the AAV, including any ITR and any nucleic acid between them, is referred to as the “vector genome.” Therefore, the term rAAV vector encompasses an rAAV viral particle that comprises a capsid and a heterologous nucleic acid, i.e., a nucleic acid not originally present in the capsid in nature, and hereinafter referred to as a “vector genome.” Thus, a “rAAV vector genome” (or “vector genome”) refers to a heterologous polynucleotide sequence (including at least one ITR, typically, but not necessarily, an ITR not associated with the original nucleic acid present in the original AAV) that may, but need not, be contained within an AAV capsid. An rAAV vector genome may be double-stranded (dsAAV), single-stranded (ssAAV) and/or self-complementary (scAAV).

As used herein, the terms “rAAV vector,” “rAAV viral particle” and/or “rAAV vector particle” refer to an AAV capsid comprised of at least one AAV capsid protein (though typically all of the capsid proteins, e.g, VPI, VPS and VP3, or variant thereof, of an AAV are present) and containing a vector genome comprising a heterologous nucleic acid sequence not originally present in the original AAV capsid. These terms are to be distinguished from an “AAV viral particle” or “AAV virus” that is not recombinant wherein the capsid contains a virus genome encoding rep and cap genes and which AAV virus is capable of replicating if present in a cell also comprising a helper virus, such as an adenovirus and/or herpes simplex virus, and/or required helper genes therefrom. Thus, production of an rAAV vector particle necessarily includes production of a recombinant vector genome using recombinant DNA technologies, as such, which vector genome is contained within a capsid to form an rAAV vector, rAAV viral particle, or an rAAV vector particle.

The genomic sequences of various serotypes of AAV, as well as the sequences of the inverted terminal repeats (ITRs), rep proteins, and capsid subunits, both existing in nature and/or mutants and variants thereof, are known in the art. Such sequences may be found in the literature or in public databases such as GenBank. See, e.g., GenBank Accession Numbers NC-002077 (AAV-1), AF063497 (AAV-1), NC-001401 (AAV-2), AF043303 (AAV-2), NC-001729 (AAV-3), NC 001863 (AAV-3B), NC-001829 (AAV-4), U89790 (AAV-4), NC-006152 (AAV-5), AF513851 (AAV-7), AF513852 (AAV-8), and NC-006261 (AAV-8); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379, WO 2014/194132, WO 2015/121501; and U.S. Pat. Nos. 6,156,303 and 7,906,111.

As used herein, the term “ameliorate” means a detectable or measurable improvement in a subject's disease, disorder or condition, or symptom thereof, or an underlying cellular response. A detectable or measurable improvement includes a subjective or objective decrease, reduction, inhibition, suppression, limit or control in the occurrence, frequency, severity, progression or duration of, complication cause by or associated with, improvement in a symptom of, or a reversal of a disease, disorder or condition.

As used herein, the term “associated with” refers to with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example, by means of hydrogen bonds, van der Waals interaction, hydrophobic interactions, magnetism, and a combination thereof.

As used herein, the term “cis-motif” or “cis-element” includes conserved sequences such as those found at, or close to, the termini of the genomic sequence and recognized for initiation of replication; cryptic promoters or sequences at internal positions likely used for transcription initiation, splicing or termination. A cis-motif or cis-element is present on the same nucleic acid molecule as those sequences with which it interacts. This is to be distinguished from “trans-motif” sequences that act “in trans” with other sequences that are not located on the same nucleic acid molecule.

As used herein, the term “coding sequence” or “encoding nucleic acid” refers to a nucleic acid sequence which encodes a protein or polypeptide and denotes a sequence which is transcribed (in the case of DNA) and translated (in the case of mRNA) into a polypeptide in vitro or in vivo when placed under the control of (operably linked to) appropriate regulatory sequences. Boundaries of a coding sequence are generally determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

As used herein, the term “chimeric” refers to a viral capsid, with capsid sequences from different parvoviruses, preferably different AAV serotypes, as described in Rabinowitz et al., U.S. Pat. No. 6,491,907, the disclosure of which is incorporated in its entirety herein by reference. See also Rabinowitz et al. (2004) J. Virol. 78(9):4421-4432. In some embodiments, a chimeric viral capsid is an AAV2.5 capsid which has the sequence of the AAV2 capsid with the following mutations: 263 Q to A; 265 insertion T; 705 N to A; 708 V to A; and 716 T to N. The nucleotide sequence encoding such capsid is defined as SEQ ID NO: 15 as described in WO 2006/066066. Other preferred chimeric AAV capsids include, but are not limited to, AAV2i8 described in WO 2010/093784, AAV2G9 and AAV8G9 described in WO 2014/144229, and AAV9.45 (Pulicherla et al. (2011) Molecular Therapy 19(6):1070-1078), AAV-NP4, NP22, NP66, AAV-LK01 through AAV-LK019 described in WO 2103/029030, RHM4-1 and RHM15-1 through RHMS-6 described in WO 205/013313, AAV-DJ, AAV-DJ/8, AAV-DJ/9 described in WO 2007/120542.

As used herein, the term “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or are of a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid, glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine. A “conservative substitution” also includes the use of a substituted amino acid in place of an unsubstituted parent amino acid.

As used herein, the term “flanked,” refers to a sequence that is flanked by other elements and indicates the presence of one or more flanking elements upstream and/or downstream, i.e., 5′ and/or 3′, relative to the sequence. The term “flanked” is not intended to indicate that the sequences are necessarily contiguous. For example, there may be intervening sequences between a nucleic acid encoding a transgene and a flanking element. A sequence (e.g., a transgene) that is “flanked” by two other elements (e.g., ITRs), indicates that one element is located 5′ to the sequence and the other is located 3′ to the sequence; however, there may be intervening sequences there between.

As used herein, the term “fragment” refers to a material or entity that has a structure that includes a discrete portion of the whole but lacks one or more moieties found in the whole. In some embodiments, a fragment consists of a discrete portion. In some embodiments, a fragment consists of or comprises a characteristic structural element or moiety found in the whole. In some embodiments, a polymer fragment comprises, or consists of, at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., amino acid residues, nucleotides) found in the whole polymer.

As used herein, the term “functional” refers to a biological molecule in a form in which it exhibits a property and/or activity by which it is characterized. A biological molecule may have two functions (i.e., bifunctional) or many functions (i.e., multifunctional).

As used herein, the term “gene” refers to a polynucleotide containing at least one open reading frame that is capable of encoding a particular polypeptide or protein after being transcribed and translated. “Gene transfer” or “gene delivery” refers to methods or systems for reliably inserting foreign DNA into host cells. Such methods can result in transient expression of non-integrated transferred DNA, extrachromosomal replication and expression of transferred replicons (e.g. episomes), and/or integration of transferred genetic material into the genomic DNA of host cells.

As used herein, the term “heterologous” or “exogenous” nucleic acid refers to a nucleic acid inserted into a vector (e.g., rAAV vector) for purposes of vector mediated transfer/delivery of the nucleic acid into a cell. Heterologous nucleic acids are typically distinct from the vector (e.g., AAV) nucleic acid, that is, the heterologous nucleic acid is non-native with respect to the viral (e.g., AAV) nucleic acid found in the AAV in nature. Once transferred (e.g., transduced) or delivered into a cell, a heterologous nucleic acid, contained within a vector, can be expressed (e.g., transcribed and translated if appropriate). Alternatively, a transferred (transduced) or delivered heterologous nucleic acid in a cell, contained within the vector, need not be expressed. Although the term “heterologous” is not always used herein in reference to a nucleic acid, reference to a nucleic acid even in the absence of the modifier “heterologous” is intended to include a heterologous nucleic acid. For example, a heterologous nucleic acid would be a nucleic acid encoding an ASPA polypeptide, for example a codon optimized nucleic acid encoding ASPA used in the treatment of Canavan disease.

As used herein, the term “homologous,” or “homology,” refers to two or more reference entities (e.g., a nucleic acid or polypeptide sequence) that share at least partial identity over a given region or portion. For example, when an amino acid position in two peptides is occupied by identical amino acids, the peptides are homologous at that position. Notably, a homologous peptide will retain activity or function associated with the unmodified or reference peptide and the modified peptide will generally have an amino acid sequence “substantially homologous” with the amino acid sequence of the unmodified sequence. When referring to a polypeptide, nucleic acid or fragment thereof, “substantial homology” or “substantial similarity,” means that when optimally aligned with appropriate insertions or deletions with another polypeptide, nucleic acid (or its complementary strand) or fragment thereof, there is sequence identity in at least about 95% to 99% of the sequence. The extent of homology (identity) between two sequences can be ascertained using computer program or mathematical algorithm. Such algorithms that calculate percent sequence homology (or identity) generally account for sequence gaps and mismatches over the comparison region or area. Exemplary programs and algorithms are provided below.

As used herein, the terms “host cell,” “host cell line,” and “host cell culture” are used interchangeably and refers to a cell into which an exogenous nucleic acid has been introduced, and includes the progeny of such a cell. A host cell includes a “transfectant,” “transformant,” “transformed cell,” and “transduced cell,” which includes the primary transfected, transformed or transduced cell, and progeny derived therefrom, without regard to the number of passages. In some embodiments, a host cell is a packaging cell for production of an rAAV vector.

As used herein, the term “identity” or “identical to” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 99% or more identical.

Calculation of the percent identity of two nucleic acid or polypeptide sequences, for example, can be performed by aligning two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. Nucleotides at corresponding positions are then compared. When a position in a first sequence is occupied by the same residue (e.g., nucleotide or amino acid) as the corresponding position in a second sequence, then the molecules are identical at that position. The percent identity between two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, or homology, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Also of interest is the BestFit program using the local homology algorithm of Smith and Waterman (1981, Advances in Applied Mathematics 2: 482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

As used herein, the terms “increase,” improve” or “reduce” indicate values that are relative to a baseline measurement, such as a measurement in the same individual prior to initiation of treatment described herein, or a measurement in a control individual (or multiple control individuals) in the absence of the treatment described herein. In some embodiments, a “control individual” is an individual afflicted with the same form of disease or injury as an individual being treated.

As used herein, the terms “inverted terminal repeat,” “ITR,” “terminal repeat,” and “TR” refer to palindromic terminal repeat sequences at or near the ends of the AAV genome, comprising mostly complementary, symmetrically arranged sequences. These ITRs can fold over to form T-shaped hairpin structures that function as primers during initiation of DNA replication. They are also needed for viral genome integration into host genome, for the rescue from the host genome; and for the encapsidation of viral nucleic acid into mature virions. The ITRs are required in cis for vector genome replication and its packaging into viral particles. “5′ ITR” refer to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa. In some embodiments, at least one ITR is present at the 5′ and/or 3′ end of a recombinant vector genome such that the vector genome can be packaged into a capsid to produce an rAAV vector (also referred to herein as “rAAV vector particle” or “rAAV viral particle”) comprising the vector genome.

As used herein, the term “isolated” refers to a substance or composition that is 1) designed, produced, prepared, and or manufactured by the hand of man and/or 2) separated from at least one of the components with which it was associated when initially produced (whether in nature and/or in an experimental setting). Generally, isolated compositions are substantially free of one or more materials with which they normally associate with in nature, for example, one or more protein, nucleic acid, lipid, carbohydrate and/or cell membrane. The term “isolated” does not exclude man-made combinations, for example, a recombinant nucleic acid, a recombinant vector genome (e.g., rAAV vector genome), an rAAV vector particle (e.g., such as, but not limited to, an rAAV vector particle comprising an AAV/Olig001 capsid) that packages, e.g., encapsidates, a vector genome and a pharmaceutical formulation. The term “isolated” also does not exclude alternative physical forms of the composition, such as hybrids/chimeras, multimers/oligomers, modifications (e.g., phosphorylation, glycosylation, lipidation), variants or derivatized forms, or forms expressed in host cells that are man-made.

Isolated substances or compositions may be separated from about 10%, about 20%, about 30%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure,” after having been combined with certain other components such as, for example, one or more carriers or excipients (e.g., buffer, solvent, water, etc.); in such embodiments, percent isolation or purity of the substance is calculated without including such carriers or excipients.

As used herein, the terms “nucleic acid sequence,” “nucleotide sequence,” and “polynucleotide” refer interchangeably to any molecule composed of or comprising monomeric nucleotides connected by phosphodiester linkages. A nucleic acid may be an oligonucleotide or a polynucleotide. Nucleic acid sequences are presented herein in the direction from the 5′ to the 3′ direction. A nucleic acid sequence (i.e., a polynucleotide) of the present disclosure can be a deoxyribonucleic acid (DNA) molecule or ribonucleic acid (RNA) molecule and refers to all forms of a nucleic acid such as, double stranded molecules, single stranded molecules, small or short hairpin RNA (shRNA), micro RNA, small or short interfering RNA (siRNA), trans-splicing RNA, antisense RNA, messenger RNA, transfer RNA, ribosomal RNA. Where a polynucleotide is a DNA molecule, that molecule can be a gene, a cDNA, an antisense molecule or a fragment of any of the foregoing molecules. Nucleotides are indicated herein by a single letter code: adenine (A), guanine (G), thymine (T), cytosine (C), inosine (I) and uracil (U). A nucleotide sequence may be chemically modified or artificial. Nucleotide sequences include peptide nucleic acids (PNA), morpholinos and locked nucleic acids (LNA), as well as glycol nucleic acids (GNA) and threose nucleic acids (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′-P5′-phosphoramidates, and oligoribonucleotide phosphorothioates and their 2′-O-allyl analogs and 2′-O-methylribonucleotide methylphosphonates which may be used in a nucleotide sequence of the disclosure.

As used here, the term “nucleic acid construct,” refers to a non-naturally occurring nucleic acid molecule resulting from the use of recombinant DNA technology (e.g., a recombinant nucleic acid). A nucleic acid construct is a nucleic acid molecule, either single or double stranded, which has been modified to contain segments of nucleic acid sequences, which are combined and arranged in a manner not found in nature. A nucleic acid construct may be a “vector” (e.g., a plasmid, an rAAV vector genome, an expression vector, etc.), that is, a nucleic acid molecule designed to deliver exogenously created DNA into a host cell.

As used herein, the term “operably linked” refers to a linkage of nucleic acid sequence (or polypeptide) elements in a functional relationship. A nucleic acid is operably linked when it is placed into a functional relationship with another nucleic acid sequence. For instance, a promoter or other transcription regulatory sequence (e.g., an enhancer) is operably linked to a coding sequence if it affects the transcription of the coding sequence. In some embodiments, operably linked means that nucleic acid sequences being linked are contiguous. In some embodiments, operably linked does not mean that nucleic acid sequences are contiguously linked, rather intervening sequences are between those nucleic acid sequences that are linked.

As used herein, the term “pharmaceutically acceptable” and “physiologically acceptable” refers to a biologically acceptable formulation, gaseous, liquid or solid, or mixture thereof, which is suitable for one or more routes of administration, in vivo delivery or contact.

As used herein, the terms “polypeptide,” “protein,” “peptide” or “encoded by a nucleic acid sequence” (i.e., encode by a polynucleotide sequence, encoded by a nucleotide sequence) refer to full-length native sequences, as with naturally occurring proteins, as well as functional subsequences, modified forms or sequence variants so long as the subsequence, modified form or variant retains some degree of functionality of the native full-length protein. In methods and uses of the disclosure, such polypeptides, proteins and peptides encoded by the nucleic acid sequences can be but are not required to be identical to the endogenous protein that is defective, or whose expression is insufficient, or deficient in a subject treated with gene therapy.

As used herein, the term “prevent” or “prevention” refers to delay of onset, and/or reduction in frequency and/or severity of one or more sign or symptom of a particular disease, disorder or condition (e.g., Canavan disease). In some embodiments, prevention is assessed on a population basis such that an agent is considered to “prevent” a particular disease, disorder or condition if a statistically significant decrease in the development, frequency and/or intensity of one or more sign or symptom of the disease, disorder or condition is observed in a population susceptible to the disease, disorder or condition. Prevention may be considered complete when onset of disease, disorder or condition has been delayed for a predefined period of time.

As used herein, the term “recombinant,” refers to a vector, polynucleotide (e.g., a recombinant nucleic acid), polypeptide or cell that is the product of various combinations of cloning, restriction or ligation steps (e.g. relating to a polynucleotide or polypeptide comprised therein), and/or other procedure that results in a construct that is distinct from a product found in nature. A recombinant virus or vector (e.g, rAAV vector) comprises a vector genome comprising a recombinant nucleic acid (e.g., a nucleic acid comprising a transgene and one or more regulatory elements, e.g., a codon optimized nucleic acid encoding ASPA and a CBh promoter). The terms respectively include replicates of the original polynucleotide construct and progeny of the original virus construct.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a subject is a nur7 mouse. In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is suffering from a disease, disorder or condition associated with deficient or dysfunctional aspartoacylase activity, e.g., Canavan disease. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered (e.g., gene therapy for Canavan disease). In some embodiments, a subject is a human patient with Canavan disease.

As used herein, the term “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve or an absolute result. The term “substantially” is therefore used herein to capture the potential lack of completeness inherent in many biological and chemical phenomena.

As used herein, the term “symptoms are reduced” or “reduce symptoms” refers to when one or more symptoms of a particular disease, disorder or condition is reduced in magnitude (e.g., intensity, severity etc.) and/or frequency. For purposes of clarity, a delay in the onset of a particular symptom is considered one form of reducing the frequency of that symptom.

As used herein, the term “therapeutic polypeptide” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a host cell, is an enzyme expressed from a transgene (i.e., an exogenous nucleic acid that has been introduced into the host cell). In some embodiments, a therapeutic polypeptide is an ASPA protein expressed from a therapeutic transgene transduced into a cerebral cortical cell (e.g., an oligodendrocyte).

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector). A transgene may be operably linked to a control sequence, such as a promoter. It will be appreciated by those of skill in the art that expression control sequences can be selected based on ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. An example of a transgene is a nucleic acid encoding a therapeutic polypeptide, for example an ASPA polypeptide, and an exemplary promoter is one not operable linked to a nucleotide encoding ASPA in nature. Such a non-endogenous promoter can include a CBh promoter, among many others known in the art.

A nucleic acid of interest can be introduced into a host cell by a wide variety of techniquest that are well-known in the art, including transfection and transduction.

“Transfection” is generally known as a technique for introducing an exogenous nucleic acid into a cell without the use of a viral vector. As used herein, the term “transfection” refers to transfer of a recombinant nucleic acid (e.g., an expression plasmid) into a cell (e.g., a host cell) without use of a viral vector. A cell into which a recombinant nucleic acid has been introduced is referred to as a “transfected cell.” A transfected cell may be a host cell (e.g., a CHO cell, Pro10 cell, HEK293 cell) comprising an expression plasmid/vector for producing a recombinant AAV vector. In some embodiments, a transfected cell (e.g., a packing cell) may comprise a plasmid comprising a transgene (e.g., an ASPA transgene), a plasmid comprising an AAV rep gene and an AAV cap gene and a plasmid comprising a helper gene. Many transfection techniques are known in the art, which include, but are not limited to, electroporation, calcium phosphate precipitation, microinjection, cationic or anionic liposomes, and liposomes in combination with a nuclear localization signal.

As used herein, the term “transduction” refers to transfer of a nucleic acid (e.g., a vector genome) by a viral vector (e.g., rAAV vector) to a cell (e.g., a target cell, including, but not limited to, an oligodendrocyte). In some embodiments, a gene therapy for Canavan disease includes transducing a vector genome comprising a modified nucleic acid encoding ASPA into an oligodendrocyte. A cell into which a transgene has been introduced by a virus or a viral vector is referred to as a “transduced cell.” In some embodiments, a transduced cell is an isolated cell and transduction occurs ex vivo. In some embodiments, a transduced cell is a cell within an organism (e.g., a subject) and transduction occurs in vivo. A transduced cell may be a target cell of an organism which has been transduced by a recombinant AAV vector such that the target cell of the organism expresses a polynucleotide (e.g., a transgene, e.g., a modified nucleic acid encoding ASPA).

Cells that may be transduced include a cell of any tissue or organ type, or any origin (e.g., mesoderm, ectoderm or endoderm). Non-limiting examples of cells include liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells or hematopoietic (e.g., blood or lymph) cells. Additional examples include stem cells, such as pluripotent or multipotent progenitor cells that develop or differentiate into liver (e.g., hepatocytes, sinusoidal endothelial cells), pancreas (e.g., beta islet cells, exocrine cells), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblast, myocytes), synoviocytes, chondrocytes, osteoclasts, epithelial cells, endothelial cells, salivary gland cells, inner ear nervous cells or hematopoietic (e.g., blood or lymph) cells.

In some embodiments, cells present within particular areas of a tissue or organ (e.g., brain) may be transduced by an rAAV vector (e.g., an rAAV comprising an ASPA transgene) that is administered to the tissue or organ. In some embodiments, a brain cell is transduced with an rAAV comprising an ASPA transgene. In some embodiments, a cell of the cortex of the brain is transduced with an rAAV comprising an ASPA transgene. In some embodiments, a cell of the striatum of the brain is transduced with an rAAV comprising an ASPA transgene. In some embodiments, a subcortical white matter cell of the brain is transduced with an rAAV comprising an ASPA transgene. In some embodiments, a cell of the cerebellum of the brain is transduced with rAAV comprising an ASPA transgene.

As used herein, the terms “treat,” “treating” or treatment refer to administration of a therapy that partially or completely alleviates, ameliorates, relieves, inhibits, delays onset of, reduces severity of, and/or reduces incidence of one or more symptoms, features, and/or causes of a particular disease, disorder, and/or condition.

As used herein, the term “vector” refers to a plasmid, virus (e.g., an rAAV), cosmid, or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) sequence and/or an ITR. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence. In some embodiments, when delivered to a host cell, the nucleic acid sequence, or a portion of the nucleic acid sequence is packaged into a capsid. A host cell may be an isolated cell or a cell within a host organism. In addition to a nucleic acid sequence (e.g., transgene) which encodes a polypeptide or protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene. Plasmid vectors may be referred to herein as “expression vectors.”

As used herein, the term “vector genome” refers to a recombinant nucleic acid sequence that is packaged or encapsidated to form an rAAV vector. Typically, a vector genome includes a heterologous polynucleotide sequence, e.g., a transgene, regulatory elements, ITRs not originally present in the capsid. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is typically referred to as the “plasmid backbone,” which is important for cloning. selection and amplification of the plasmid, a process that is needed for propagation of recombinant viral vector production, but which is not itself packaged or encapsidated into an rAAV vector.

As used herein, the term “viral vector” generally refers to a viral particle that functions as a nucleic acid delivery vehicle and which comprises a vector genome (e.g., comprising a transgene instead of a nucleic acid encoding an AAV rep and cap) packaged within the viral particle (i.e., capsid) and includes, for example, lenti- and parvo-viruses, including AAV serotypes and variants (e.g., rAAV vectors). A recombinant viral vector does not comprise a vector genome comprising a rep and/or a cap gene.

The present disclosure provides modified nucleic acids comprising a modified ASPA coding sequence, and use thereof, in gene therapy pharmaceutical compositions. By “modified,” as used herein, is meant that the nucleic acid sequence encoding a polypeptide that exists in nature has been altered such that, in one embodiment, the modified nucleic acid sequence drives a higher level of expression of the protein in a cell compared with the level of expression of the protein from the unmodified, i.e., occurring in nature (including mutant forms of a gene), nucleic acid sequence in an otherwise identical cell. The disclosure also provides recombinant nucleic acids, including vector genomes, which include as part of their sequence, a modified ASPA coding sequence. Further, the disclosure provides for packaged gene delivery vehicles, such as an rAAV vector, which includes the modified ASPA coding sequence. The disclosure also includes methods of delivery and, preferably, expression of the modified ASPA coding sequence in a cell. The disclosure also provides gene therapy methods in which the modified ASPA coding sequence is administered to a subject, e.g., as a component of a vector and/or packaged as a component of a viral gene delivery vehicle (e.g., an rAAV vector). Treatment may, for example, be effected to increase levels of ASPA in a subject and to treat an ASPA deficiency in a subject. Each of these aspects of the disclosure is discussed further in the ensuing sections.

AAV and rAAV Vectors

AAV

As discussed supra, the terms “adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g, rAAV vector genome) forms circular concatemers that persist as episomes in the nucleus of transduced cells. In some embodiments, a transgene is inserted in specific sites in the host cell genome, for example at a site on human chromosome 19. Site-specific integration, as opposed to random integration, is believed to likely result in a predictable long-term expression profile. The insertion site of AAV into the human genome is referred to as AAVS1. Once introduced into a cell, polypeptides encoded by the nucleic acid can be expressed by the cell. Because AAV is not associated with any pathogenic disease in humans, a nucleic acid delivered by AAV can be used to express a therapeutic polypeptide for the treatment of a disease, disorder and/or condition in a human subject.

Multiple serotypes of AAV exist in nature with at least fifteen wild type serotypes having been identified from humans thus far (i.e., AAV1-AAV15). Naturally occurring and variant serotypes are distinguished by having a protein capsid that is serologically distinct from other AAV serotypes. AAV type 1 (AAV1), AAV type 2 (AAV2), AAV type 3 (AAV3) including AAV type 3A (AAV3A) and AAV type 3B (AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 12 (AAV12), AAVrh10, AAVrh74 (see WO 2016/210170), avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV type 2i8 (AAV2i8), NP4, NP22, NP66, DJ, DJ/8, DJ/9, LK3, RHM4-1, among many others. “Primate AAV” refers to AAV that infect primates, “non-primate AAV” refers to AAV that infect non-primate mammals, “bovine AAV” refers to AAV that infect bovine mammals, and so on. Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, e.g., AAV, as well as viruses, e.g., AAV, that are not serologically distinct but that may be within a subgroup or a variant of a given serotype.

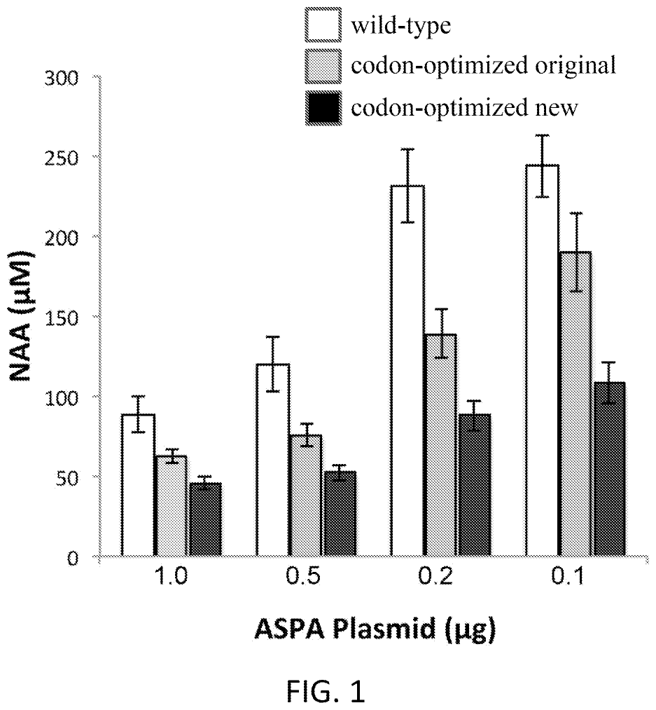

A comprehensive list and alignment of amino acid sequences of capsids of known AAV serotypes is provided by Marsic et al. (2014) Molecular Therapy 22(11):1900-1909, especially at supplementary FIG. 1.

Genomic sequences of various serotypes of AAV, as well as sequences of the native terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001863 (AAV3B), NC_001829 (AAV4), U89790 (AAV4), NC_006152 (AAV5), NC_001862 (AAV6), AF513851 (AAV7), AF513852 (AAV8), and NC_006261 (AAV8); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111. For illustrative purposes only, wild type AAV2 comprises a small (20-25 nm) icosahedral virus capsid of AAV composed of three proteins (VP1, VP2, and VP3; a total of 60 capsid proteins compose the AAV capsid) with overlapping sequences. The proteins VP1 (735 aa; Genbank Accession No. AAC03780), VP2 (598 aa; Genbank Accession No. AAC03778) and VP3 (533 aa; Genbank Accession No. AAC03779) exist in a 1:1:10 ratio in the capsid. That is, for AAVs, VP1 is the full length protein and VP2 and VP3 are progressively shorter versions of VP1, with increasing truncation of the N-terminus relative to VP1.

Recombinant AAV

As discussed supra, a “recombinant adeno-associated virus” or “rAAV” is distinguished from a wild-type AAV by replacement of all or part of the endogenous viral genome with a non-native sequence. Incorporation of a non-native sequence within the virus defines the viral vector as a “recombinant” vector, and hence a “rAAV vector.” An rAAV vector can include a heterologous polynucleotide encoding a desired protein or polypeptide (e.g., ASPA polypeptide). A recombinant vector sequence may be encapsidated or packaged into an AAV capsid and referred to as an “rAAV vector,” an “rAAV vector particle,” “rAAV viral particle” or simply a “rAAV.”

For the production of an rAAV vector, the desired ratio of VP1:VP2:VP3 is in the range of about 1:1:1 to about 1:1:100, preferably in the range of about 1:1:2 to about 1:1:50, more preferably in the range of about 1:1:5 to about 1:1:20. Although the desired ratio of VP1:VP2 is 1:1, the ratio range of VP1:VP2 could vary from 1:50 to 50:1.

The present disclosure provides for an rAAV vector comprising a polynucleotide sequence not of AAV origin (e.g., a polynucleotide heterologous to AAV). The heterologous polynucleotide may be flanked by at least one, and sometimes by two, AAV terminal repeat sequences (e.g., inverted terminal repeats (ITRs)). The heterologous polynucleotide flanked by ITRs, also referred to herein as a “vector genome,” typically encodes a polypeptide of interest, or a gene of interest (“GOI”), such as a target for therapeutic treatment (e.g., a nucleic acid encoding ASPA for the treatment of Canavan disease). Delivery or administration of an rAAV vector to a subject (e.g. a patient) provides encoded proteins and peptides to the subject. Thus, an rAAV vector can be used to transfer/deliver a heterologous polynucleotide for expression for, e.g., treating a variety of diseases, disorders and conditions.

rAAV vector genomes generally retain 145 base ITRs in cis to the heterologous nucleic acid sequence that replaced the viral rep and cap genes. Such ITRs are necessary to produce a recombinant AAV vector; however, modified AAV ITRs and non-AAV terminal repeats including partially or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. An rAAV vector genome optionally comprises two ITRs which are generally at the 5′ and 3′ ends of the vector genome comprising a heterologous sequence (e.g., a transgene encoding a gene of interest, or a nucleic acid sequence of interest including, but not limited to, an antisense, and siRNA, a CRISPR molecule, among many others). A 5′ and a 3′ ITR may both comprise the same sequence, or each may comprise a different sequence. An AAV ITR may be from any AAV including by not limited to serotypes 1, 2, 3, 4, 5, 6, 7, 8, 9, 10 or 11 or any other AAV.

An rAAV vector of the disclosure may comprise an ITR from an AAV serotype (e.g., wild-type AAV2, a fragment or variant thereof) that differs from the serotype of the capsid (e.g., AAV8, Olig001). Such an rAAV vector comprising at least one ITR from one serotype, but comprising a capsid from a different serotype, may be referred to as a hybrid viral vector (see U.S. Pat. No. 7,172,893). An AAV ITR may include the entire wild type ITR sequence, or be a variant, fragment, or modification thereof, but will retain functionality.

In some embodiments, a heterologous polypeptide comprises an ITR (e.g., an ITR from AAV2, but can comprise an ITR from any wild type AAV serotype, or a variant thereof) positioned at the left and right ends (i.e., 5′ and 3′ termini, respectively) of a vector genome. In some embodiments, a left (e.g., 5′) ITR comprises or consists of the nucleic acid sequence of SEQ ID NO:5, SEQ ID NO:12 or SEQ ID NO:19. In some embodiments, a left (e.g., 5′) ITR comprises a nucleic acid sequence that is about 80%, about 85%, about 90%, about 95%, about 98%, about 99% or 100% identical to SEQ ID NO:5, SEQ ID NO:12 or SEQ ID NO:19. In some embodiments, a right (e.g., 3′) ITR comprises or consists of a nucleic acid sequence of SEQ ID NO:5, SEQ ID NO:12 or SEQ ID NO:19. In some embodiments, a right (e.g., 3′) ITR comprises a nucleic acid sequence that is about 80%, about 85%, about 90%, about 95%, about 98%, about 99% or 100% identical to SEQ ID NO:5, SEQ ID NO:12 or SEQ ID NO:19. Each ITR is in cis with but may be separated from each other, or other elements in the vector genome, by a nucleic acid sequence of variable length, such as a recombinant nucleic acid comprising a modified nucleic acid encoding ASPA and regulatory elements. In some embodiments, ITRs are AAV2 ITRs, or variants thereof, and flank an ASPA transgene. In some embodiments, an rAAV comprises an ASPA transgene (e.g., comprising the nucleic acid sequence of SEQ ID NO:2) flanked by AAV2 ITRs (e.g., ITRs having the sequence as set forth in SEQ ID NO:5, SEQ ID NO:12 or SEQ ID NO:19).

In some embodiments, an rAAV vector genome is linear, single-stranded and flanked by AAV ITRs. Prior to transcription and translation of the heterologous gene, a single stranded DNA genome of approximately 4700 nucleotides must be converted to a double-stranded form by DNA polymerases (e.g., DNA polymerases within the transduced cell) using the free 3′-OH of one of the self-priming ITRs to initiate second-strand synthesis. In some embodiments, full length-single stranded vector genomes (i.e., sense and anti-sense) anneal to generate a full length-double stranded vector genome. This may occur when multiple rAAV vectors carrying genomes of opposite polarity (i.e., sense or anti-sense) simultaneously transduce the same cell. Regardless of how they are produced, once double-stranded vector genomes are formed, the cell can transcribe and translate the double-stranded DNA and express the heterologous gene.

The efficiency of transgene expression from an rAAV vector can be hindered by the need to convert a single stranded rAAV genome (ssAAV) into double-stranded DNA prior to expression. This step is circumvented by using a self-complementary AAV genome (scAAV) that can package an inverted repeat genome that can fold into double-stranded DNA without the need for DNA synthesis or base-pairing between multiple vector genomes (McCarty, (2008) Molec. Therapy 16(10):1648-1656; McCarty et al., (2001) Gene Therapy 8:1248-1254; McCarty et al., (2003) Gene Therapy 10:2112-2118). A limitation of a scAAV vector is that size of the unique transgene, regulatory elements and IRTs to be package in the capsid is about half the size (i.e., ˜2,500 nucleotides of which 2,200 nucleotides may be a transgene and regulatory elements, plus two copies of the ˜145 nucleotide ITRs) of a ssAAV vector genome (i.e., ˜4,900 nucleotides including two ITRs).

scAAV vector genomes are made by using a nucleic acid not comprising the terminal resolution site (TRS), or by altering the TRS, from one rAAV ITR of a vector, e.g, a plasmid, comprising the vector genome thereby preventing initiation of replication from that end (see U.S. Pat. No. 8,784,799). AAV replication within a host cell is initiated at the wild type ITR of the scAAV vector genome and continues through the ITR lacking or comprising an altered terminal resolution site and then back across the genome to create a complementary strand. The resulting complementary single nucleic acid molecule is thus a self-complementary nucleic acid molecule that results in a vector genome with a mutated (is not resolved) ITR in the middle, and wild-type ITRs at each end. In some embodiments, a mutant ITR lacking a TRS or comprising an altered TRS is at the 5′ end of the vector genome. In some embodiments, a mutant ITR lacking a TRS or comprising an altered TRS that is not resolved (cleaved) is at the 3′ end of the vector genome. In some embodiments, a mutant ITR comprises the nucleic acid of SEQ ID NO:5, SEQ ID NO:12 or SEQ ID NO:19.

Without wishing to be bound by theory, while the two halves of a scAAV genome are complementary, it is unlikely that there is substantial base pairing within the capsid as many of the bases are in contact with amino acid residues of the inner capsid shell and the phosphate backbone is sequestered toward the center (McCarty, Molec. Therapy (2008) 16(10):1648-1656). It likely that upon uncoating, the two halves of the scAAV genome anneal to form a dsDNA hairpin molecule, with a covalently closed ITR at one end and two open-ended ITRs on the other. The ITRs flank a double-stranded region encoding, among other things, the transgene, and regulatory elements in cis thereto.

A viral capsid of an rAAV vector may be from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., VIROLOGY, volume 2, chapter 69 (4^{th} ed., Lippincott-Raven Publishers). Capsids may be derived from a number of AAV serotypes disclosed in U.S. Pat. No. 7,906,111; Gao et al. (2004) J. Virol. 78:6381; Morris et al. (2004) Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313. One skilled in the art would know there are likely other AAV variants not yet identified that perform the same or similar function. A full complement of AAV cap proteins includes VP1, VP2, and VP3. The ORF comprising nucleotide sequences encoding AAV VP capsid proteins may comprise less than a full complement AAV Cap proteins or the full complement of AAV cap proteins may be provided.

In another embodiment, the present disclosure provides for the use of ancestral AAV vectors for use in therapeutic in vivo gene therapy. Specifically, in silico-derived sequences may be synthesized de novo and characterized for biological activities. Prediction and synthesis of ancestral sequences, in addition to assembly into an rAAV vector, may be accomplished using methods described in WO 2015/054653, the contents of which are incorporated by reference herein. Notably, rAAV vectors assembled from ancestral viral sequences may exhibit reduced susceptibility to pre-existing immunity in human populations as compared to contemporary viruses or portions thereof.

In some embodiments, an rAAV vector comprising a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz et al. (2002) J. Virology 76(2):791-801). Alternatively, a chimeric capsid can comprise a mixture of a VP1 from one serotype, a VP2 from a different serotype, a VP3 from yet a different serotype, and a combination thereof. For example a chimeric virus capsid may include an AAV1 cap protein or subunit and at least one AAV2 cap protein or subunit. A chimeric capsid can, for example include an AAV capsid with one or more B19 cap subunits, e.g., an AAV cap protein or submint can be replaced by a B19 cap protein or subunit. For example, in one embodiment, a VP3 subunit of an AAV capsid can be replaced by a VP2 subunit of B19. In some embodiments, a chimeric capsid is an Olig001 capsid as described in WO2014052789 and incorporated herein by reference.

In some embodiments, chimeric vectors have been engineered to exhibit altered tropism or tropism for a particular tissue or cell type. The term “tropism” refers to preferential entry of the virus into certain cell or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., an rAAV vector genome) are expressed.

A “tropism profile” refers to a pattern of transduction of one or more target cells in various tissues and/or organs. For example, a chimeric AAV capsid may have a tropism profile characterized by efficient transduction of oligodendrocytes with only low transduction of neurons, astrocytes and other CNS cells. See WO2014/052789, incorporated herein by reference. Such a chimeric capsid may be considered “specific for oligodendrocytes” exhibiting tropism for oligodendrocytes, and referred to herein as “oligotropism,” if when administered directly into the CNS, preferentially transduces oligodendrocytes over neurons, astrocytes and other CNS cell types. In some embodiments, at least about 80% of cells that are transduced by a capsid specific for oligodendrocytes are oligodendrocytes, e.g., at least about 85%, 90%, 95%, 96%, 97%, 98% 99% or more of the transduced cells are oligodendrocytes.

In some embodiments, an rAAV vector is useful for treating or preventing a “disorder associated with oligodendrocyte dysfunction.” As used herein, the term “associated with oligodendrocyte dysfunction” refers to a disease, disorder or condition in which oligodendrocytes are damaged, lost or function improperly compared to otherwise identical normal oligodendrocytes. The term includes diseases, disorders and conditions in which oligodendrocytes are directly affected as well as diseases, disorders or conditions in which oligodendrocytes become dysfunctional secondary to damage to other cells. In some embodiments, a disorder associated with oligodendrocyte dysfunction is Canavan disease (CD).

In some embodiments, a chimeric AAV capsid with tropism for oligodendrocytes is Olig001 (also known as BNP61) and comprises sequences from AAV1, AAV2, AAV6, AAV8 and AAV9 (see WO 2014/052789). In some embodiments, the Oligo001 capsid VP1 is encoded by a nucleic acid sequence comprising or consisting of the nucleic acid sequence of SEQ ID NO:13. In some embodiments, the Olig001 capsid VP1 is encoded by a nucleic acid sequence at least 90%, at least 91%, at least 92%, at least 93%, at least 94%, at least 95%, at least 96%, at least 97%, at least 98%, at least 99% or 100% identical to the nucleic acid sequence of SEQ ID NO:13.

Nucleic acid sequences encode overlapping AAV capsid proteins, VP1, VP2 and VP3. The amino acid sequence of the Olig001 capsid proteins is set forth in SEQ ID NO:14 with VP1 starting at amino acid residue 1 (methionine), VP2 starting at amino acid residue 148 (threonine) and VP3 starting at amino acid residue 203 (methionine) of SEQ ID NO:14.

In some embodiments, a chimeric AAV capsid with tropism for oligodendrocytes is Olig002 (also known as BNP62) or Olig003 (also known as BNP63) (see WO 2014/052789). In some embodiments, the Oligo002 capsid VP1 comprises or consists of the amino acid sequence of SEQ ID NO:15. In some embodiments, the Olig002 capsid VP1 amino acid sequence is at least 90%, at least 91%, at least 92%, at least 93%, at least 94%, at least 95%, at least 96%, at least 97%, at least 98%, at least 99% or 100% identical to the sequence of SEQ ID NO:15. In some embodiments, a nucleic acid comprises a sequence encoding the amino acid sequence of SEQ ID NO:15. In some embodiments, the Oligo003 capsid comprises or consists of the amino acid sequence of SEQ ID NO:16. In some embodiments, the Olig003 capsid VP1 amino acid sequence is at least 90%, at least 91%, at least 92%, at least 93%, at least 94%, at least 95%, at least 96%, at least 97%, at least 98%, at least 99% or 100% identical to SEQ ID NO:16. In some embodiments, a nucleic acid comprises a sequence encoding the amino acid sequence of SEQ ID NO:16.

In some embodiments, an rAAV vector comprising a chimeric AAV capsid (e.g., Olig001) and a therapeutic transgene may be used to treat a disease, disorder or condition associated with oligodendrocyte dysfunction. In such a disease, disorder or condition, oligodendrocytes are damaged, lost or function improperly. This may be the result of a direct effect on the oligodendrocyte or result when oligodendrocytes become dysfunctional secondary to damage to other cells. In some embodiments, an rAAV vector comprising an AAV/Olig001 capsid and a modified ASPA nucleic acid is used to treat Canavan disease.

### Recombinant Nucleic Acids

Recombinant nucleic acids of the present disclosure include modified nucleic acids as well as plasmids and vector genomes that comprise a modified nucleic acid. A recombinant nucleic acid, plasmid or vector genome may comprise regulatory sequences to modulate propagation (e.g., of a plasmid) and/or control expression of a modified nucleic acid (e.g., a transgene). Recombinant nucleic acids may also be provided as a component of a viral vector (e.g., an rAAV vector). Generally, a viral vector includes a vector genome comprising a recombinant nucleic acid packaged in a capsid.

Modified Nucleic Acids

A modified, or variant form, of a gene, nucleic acid or polynucleotide (e.g., a transgene) refers to a nucleic acid that deviates from a reference sequence. A reference sequence may be a naturally occurring, wild type sequence (e.g., a gene) and may include naturally occurring variants (e.g., splice variants, alternative reading frames). Those skilled in the art will be aware that reference sequences can be found in publicly available databases such as GenBank (ncbi.nlm.nih.gov/genbank). Modified/variant nucleic acids may have substantially the same, greater or lesser activity, function or expression as compared to a reference sequence. Preferably, a modified, or variant nucleic acid, as used interchangeably herein, exhibits improved protein expression, e.g., a protein encoded thereby is expressed at a detectably greater level in a cell compared with the level of expression of a protein provided by an endogenous gene (e.g., a wild type gene, a mutant gene) in an otherwise identical cell. In some embodiments, a modified, or variant nucleic acid (e.g., a modified nucleic acid encoding ASPA), as used interchangeably herein, exhibits improved protein expression, e.g., a protein encoded thereby is expressed at a detectably greater level in a cell compared with the level of expression of a protein provided by an endogenous gene comprising a mutation in an otherwise identical cell.

Modifications to nucleic acids include one or more nucleotide substitutions (e.g., substitution of 1-3, 3-5, 5-10, 10-15, 15-20, 20-25, 25-30, 30-40, 40-50, 50-100 or more nucleotides), additions (e.g., insertion of 1-3, 3-5, 5-10, 10-15, 15-20, 20-25, 25-30, 30-40, 40-50, 50-100 or more nucleotides), deletions (e.g., deletion of 1-3, 3-5, 5-10, 10-15, 15-20, 20-25, 25-30, 30-40, 40-50, 50-100 or more nucleotides, deletion of a motif, domain, fragment, etc.) of a reference sequence. A modified nucleic acid may be about 50%, about 60%, about 70%, about 80%, about 85%, about 90%, about 92%, about 93%, about 94%, about 95%, about 96% about 97% about 98% or about 99% identical to a reference sequence.

A modified nucleic acid may encode a polypeptide with about 50%, about 60%, about 70%, about 80%, about 85%, about 90%, about 95%, about 98%, about 99% or 100% identity to a reference polypeptide. In some embodiments, a modified nucleic acid encoding ASPA (e.g., SEQ ID NO:2) encodes a polypeptide with 100% identify to a reference polypeptide (e.g., SEQ ID NO:4).

In some embodiments, a modified nucleic acid (e.g., transgene) encodes a wild-type protein. Such modified nucleic acid may be codon optimized. “Optimized” or “codon-optimized,” as referred to interchangeably herein, refers to a coding sequence that has been optimized relative to a wild type coding sequence or reference sequence (e.g., a coding sequence for ASPA polypeptide) to increase expression of the polypeptide, e.g., by minimizing usage of rare codons, decreasing the number of CpG dinucleotides, removing cryptic splice donor or acceptor sites, removing Kozak sequences, removing ribosomal entry sites, and the like. In some embodiments, a level of expression of a protein from a codon-optimized sequence (e.g., a modified nucleic acid encoding ASPA) is increased as compared to a level of expression of a protein from a wild type gene in an otherwise identical cell. In some embodiments, a level of expression of a protein from a codon-optimized sequence (e.g., a modified nucleic acid encoding ASPA) is not increased (e.g., expression is substantially similar) as compared to a level of expression of a protein from a wild-type gene in an otherwise identical cell. In some embodiments, a level of expression of a protein from a codon-optimized sequence (e.g., a modified nucleic acid encoding ASPA) is increased as compared to a level of expression of a protein from a mutant gene in an otherwise identical cell.

Examples of modifications include elimination of one or more cis-acting motifs and introduction of one or more Kozak sequences. In some embodiments, one or more cis-acting motifs are eliminated and one or more Kozak sequences are introduced.

Examples of cis-acting motifs that may be eliminated include internal TATA-boxes; chi-sites; ribosomal entry sites; ARE, INS, and/or CRS sequence elements; repeat sequences and/or RNA secondary structures; (cryptic) splice donor and/or acceptor sites, branch points; and restriction sites.

In some embodiments, a modified nucleic acid encodes a modified or variant polypeptide. A modified polypeptide encoded by a modified nucleic acid may retain all or a part of the function or activity of a polypeptide encoded by a wild type coding or reference sequence. In some embodiments, a modified polypeptide has one or more non-conservative or conservative amino acid changes. In some embodiments, certain domains that have been demonstrated to play a limited or no role in a function of a polypeptide are not present in a modified polypeptide (e.g., certain binding domains) (e.g., WO 2016/097219). Modified nucleic acids present in rAAV vectors may comprise fewer nucleotides than the wild type coding, or reference sequence, due to the packaging capacity of an rAAV capsid (e.g., shortened minidystrophin transgene, see WO 2001/83695; a B-domain deleted human Factor VIII transgene, see WO 2017/074526), and also include shortened transgenes that are both truncated and codon-optimized (e.g., a codon optimized mini-dystrophin transgene described in WO 2017/221145). In some embodiments, a polypeptide encoded by a modified nucleic acid has less than, the same, or greater, but at least a part of, a function or activity of a polypeptide encoded by a reference sequence.

Modified nucleic acids may have a modified GC content (e.g., the number of G and C nucleotides present in a nucleic acid sequence), a modified (e.g., increased or decreased) CpG dinucleotide content and/or a modified (e.g., increased or decreased) codon adaptation index (CAI) relative to a reference and/or wild-type sequence (e.g., a wild type ASPA coding sequence). See, e.g., WO 2017/077451 (discussing various considerations well-known in the art for codon-optimization of nucleic acid sequences of interest, including publicly available software for analyzing nucleic acid sequences for optimization). As used herein, modified refers to a decrease or an increase in a particular value, amount or effect.

In some embodiments, a GC content of a modified nucleic acid sequence of the present disclosure is increased relative to a reference and/or a wild-type gene or coding sequence. The GC content of a modified nucleic acid is at least 5%, at least 6%, at least 7%, at least 8%, at least 9%, at least 10%, at least 12%, at least 14%, at least 15%, at least 17%, at least 20%, at least 30%, at least 40%, at least 50%, at least 60%, at least 70% greater than GC content of a wild type coding sequence (e.g., SEQ ID NO:3). In some embodiments, GC content is expressed as a percentage of G (guanine) and C (cytosine) nucleotides in the sequence.

In some embodiments, a codon adaptation index of a modified nucleic acid sequence of the present disclosure is at least 0.74, at least 0.76, at least 0.77, at least 0.80, at least 0.85, at least 0.86, at least 0.87, at least 0.90, at least 0.95 or at least 0.98.

In some embodiments, a modified nucleic acid sequence of the present disclosure has a reduced level of CpG dinucleotides, that being a reduction of about 10%, 20%, 30%, 50% or more, as compared to a wild type or reference nucleic acid sequence. In some embodiments, a modified nucleic acid has 1-5 fewer, 5-10 fewer, 10-15 fewer, 15-20 fewer, 20-25 fewer, 25-30 fewer, 30-40 fewer, 40-45 fewer or 45-50 fewer, or even fewer di-nucleotides than a reference sequence (e.g., a wild type sequence).

It is known that methylation of CpG dinucleotides plays an important role in the regulation of gene expression in eukaryotes. Specifically, methylation of CpG dinucleotides in eukaryotes essentially serves to silence gene expression through interfering with the transcriptional machinery. As such, because of the gene silencing evoked by methylation of CpG motifs, nucleic acids and vectors having a reduced number of CpG dinucleotides will provide for high and longer-lasting transgene expression level.

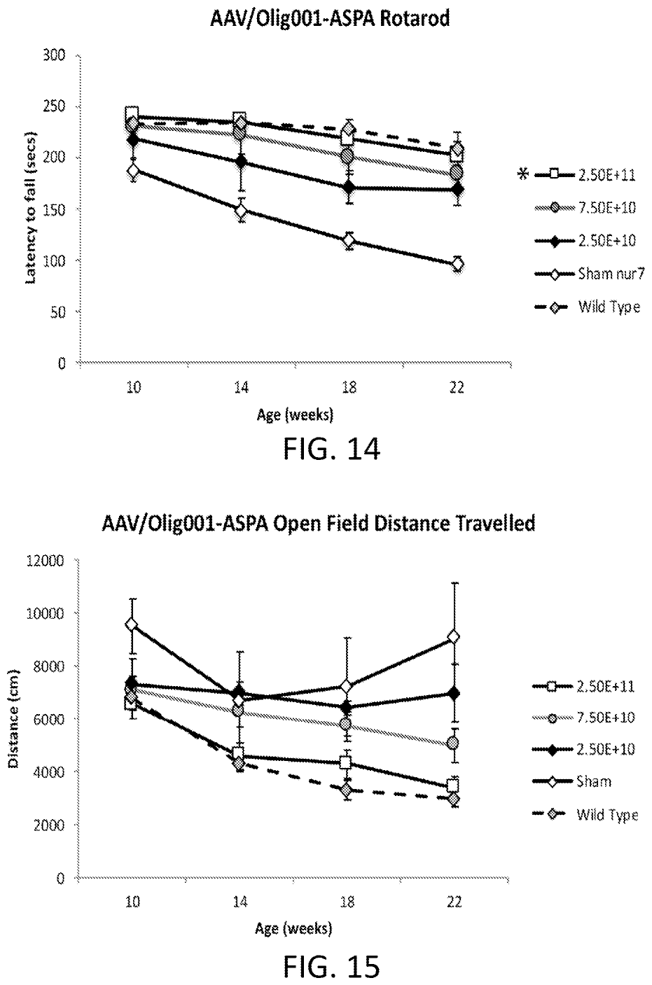

Modified nucleic acid sequences may include flanking restriction sites to facilitate subcloning into an expression vector. Many such restriction sites are well known in the art, and include, but are not limited to, those shown in FIG. 13, such as, AvaI, XmaI and XmaI.

The present disclosure includes fragments of any one of the sequences set forth in SEQ ID NOs:1-3 and which encode a functionally active fragment of the ASPA polypeptide. A “functionally active” or “functional ASPA polypeptide” indicates that the fragment provides the same or similar biological function and/or activity as a full-length ASPA polypeptide. That is, the fragment provides the same activity including, but not limited to, the ability to convert NAA to acetate and aspartate. The biological activity of ASPA, or a functional fragment thereof, also encompasses reversing or preventing the neurodegenerative phenotype associated with Canavan disease, as demonstrated elsewhere herein, and in nur7 mice.

The present disclosure provides for modified ASPA nucleic acid sequences that encode an ASPA polypeptide and which comprise at least one modification as compared with a wild type nucleic acid sequence (e.g. SEQ ID NO:3; GenBank Accession Number NM_000049.4 or NM_001128085.1, having an alternate 5′UTR but encoding for the same ASPA protein (SEQ ID NO:4)).

In some embodiments, a modified nucleic acid encoding ASPA is a codon-optimized nucleic acid encoding a wild-type ASPA polypeptide (e.g., SEQ ID NO:4) and comprises the sequence of SEQ ID NO:1 or SEQ ID NO:2. In some embodiments, a modified nucleic acid encoding ASPA is a codon-optimized nucleic acid and consists of the sequence of SEQ ID NO:1 or SEQ ID NO:2. In some embodiments, a modified nucleic acid encoding ASPA is a codon-optimized nucleic acid and comprises a sequence at least about 80%, about 85%, about 90%, about 91%, about 92%, about 93%, about 94%, about 95%, about 96%, about 97%, about 98%, about 99% or 100% identical to the sequence of SEQ ID NO:1 or SEQ ID NO:2.

In some embodiments, a cell comprising a modified nucleic acid encoding ASPA exhibits increased protein expression, e.g., the protein encoded thereby is expressed at a detectably greater level in a cell as compared with the level of expression of the protein in an otherwise identical cell comprising a wild type ASPA nucleic acid, or an otherwise identical cell comprising a mutant nucleic acid encoding ASPA. In some embodiments, a level of ASPA protein expression in a cell comprising a modified nucleic acid encoding ASPA (e.g., comprising the nucleic acid sequence of SEQ ID NO:2) is increased by about 10%, about 20%, about 30%, about 40%, about 50%, about 60%, about 70%, about 80%, about 90%, about 100%, about 120%, about 140%, about 150%, about 200%, about 300%, about 400% or more as compared to the level of ASPA protein expression in an otherwise identical cell comprising a wild-type ASPA nucleic acid. In some embodiments, the level of ASPA protein expression in a cell comprising a modified nucleic acid encoding ASPA (e.g., comprising the nucleic acid sequence of SEQ ID NO:2) is increased by about 10%, about 20%, about 30%, about 40%, about 50%, about 60%, about 70%, about 80%, about 90%, about 100%, about 120%, about 140%, about 150%, about 200%, about 300%, about 400% or more as compared to the level of ASPA protein expression in an otherwise identical cell comprising a mutant nucleic acid encoding ASPA.

In some embodiments, this can be referred to as an “expression optimized” or “enhanced expression” nucleic acid, or simply, as a “modified nucleic acid.”

One of ordinary skill would understand that a polypeptide encoded by a modified nucleic acid, and variants thereof, of the disclosure (e.g., SEQ ID NO:1, SEQ ID NO:2) is a “functional ASPA polypeptide” that provides the same or similar biological function and/or activity as a ASPA polypeptide encoded by a wild-type nucleic acid encoding ASPA (e.g., SEQ ID NO:3). That is, an ASPA polypeptide encoded by a modified nucleic acid encoding ASPA provides the same activity including, but not limited to, the ability to convert NAA to acetate and aspartate. The biological activity of ASPA encompasses reversing or preventing the neurodegenerative phenotype associated with Canavan disease as demonstrated elsewhere herein in nur7 mice including, but not limited to, improved performance of rotarod latency to fall, improved open field distance traversed, decreased NAA in brain tissue, decreased vacuole volume in the brain (e.g., thalamus, cerebellar white matter/pons), an increase in Olig2 positive cells in the brain (e.g., thalamus, cortex), and/or an increase in cortical myelination.

Regulatory Elements

The present disclosure includes a recombinant nucleic acid including a modified nucleic acid encoding ASPA and various regulatory or control elements. Typically, regulatory elements are nucleic acid sequence(s) that influence expression of an operably linked polynucleotide. The precise nature of regulatory elements useful for gene expression will vary from organism to organism and from cell type to cell type including, for example, a promoter, enhancer, intron etc., with the intent to facilitate proper heterologous polynucleotide transcription and translation. Regulatory control can be affected at the level of transcription, translation, splicing, message stability, etc. Typically, a regulatory control element that modulates transcription is juxtaposed near the 5′ end of the transcribed polynucleotide (i.e., upstream). Regulatory control elements may also be located at the 3′ end of the transcribed sequence (i.e., downstream) or within the transcript (e.g., in an intron). Regulatory control elements can be located at a distance away from the transcribed sequence (e.g., 1 to 100, 100 to 500, 500 to 1000, 1000 to 5000, 5000 to 10,000 or more nucleotides). However, due to the length of an AAV vector genome, regulatory control elements are typically within 1 to 1000 nucleotides from the polynucleotide.

Promoter

As used herein, the term “promoter,” such as a “eukaryotic promoter,” refers to a nucleotide sequence that initiates transcription of a particular gene, or one or more coding sequences (e.g., an ASPA coding sequence), in eukaryotic cells (e.g., an oligodendrocyte). A promoter can work with other regulatory elements or regions to direct the level of transcription of the gene or coding sequence(s). These regulatory elements include, for example, transcription binding sites, repressor and activator protein binding sites, and other nucleotide sequences known to act directly or indirectly to regulate the amount of transcription from the promoter, including, for example, attenuators, enhances and silencers. The promoter is most often located on the same strand and near the transcription start site, 5′ of the gene or coding sequence to which it is operably linked. A promoter is generally 100-1000 nucleotides in length. A promoter typically increases gene expression relative to expression of the same gene in the absence of a promoter.

As used herein, a “core promoter” or “minimal promoter” refers to the minimal portion of a promoter sequence required to properly initiate transcription. It may include any of the following: a transcription start site, a binding site for RNA polymerase and a general transcription factor binding site. A promoter may also comprise a proximal promoter sequence (5′ of a core promoter) that contains other primary regulatory elements (e.g., enhancer, silencer, boundary element, insulator) as well as a distal promoter sequence (3′ of a core promoter).

Examples of suitable a promoter include adenoviral promoters, such as the adenoviral major late promoter; heterologous promoters, such as the cytomegalovirus (CMV) promoter; the respiratory syncytial virus promoter; the Rous Sarcoma Virus (RSV) promoter; the albumin promoter; inducible promoters, such as the Mouse Mammary Tumor Virus (MMTV) promoter; the metallothionein promoter; heat shock promoters; the α-1-antitrypsin promoter; the hepatitis B surface antigen promoter; the transferrin promoter; the apolipoprotein A-1 promoter; chicken β-actin (CBA) promoter, the elongation factor 1a promoter (EF1a), the hybrid form of the CBA promoter (CBh promoter), and the CAG promoter (cytomegalovirus early enhancer element and the promoter, the first exon, and the first intron of chicken beta-actin gene and the splice acceptor of the rabbit beta-globin gene) (Alexopoulou et al. (2008) BioMed. Central Cell Biol. 9:2); and human ASPA gene promoter. In some embodiments, a promoter is fragment or variant of the CBh promoter and comprises or consists of the nucleic acid sequence of SEQ ID NO:7.

In some embodiments of the present disclosure, a eukaryotic promoter sequence (e.g., a CBh promoter) is operably linked to a modified nucleic acid encoding ASPA. In some embodiments, a promoter comprising the nucleic acid sequence of SEQ ID NO:7 (e.g., a CBh promoter) is operably linked to a modified nucleic acid encoding ASPA. In some embodiments, a promoter comprising or consisting of a nucleic acid sequence at least 80%, at least 85%, at least 90%, at least 95%, at least 98%, at least 99% or 100% identical to the nucleic acid sequence of SEQ ID NO:7 is operably linked to a nucleic acid comprising the nucleic acid sequence of SEQ ID NO:2. In some embodiments, a promoter comprising a nucleic acid sequence at least 95% identical to the nucleic acid sequence of SEQ ID NO:7 is operably linked to a nucleic acid sequence at least 95% identical to the nucleic acid sequence of SEQ ID NO:2 and induces expression of a polypeptide encoded by the nucleic acid sequence of SEQ ID NO:2 in oligodendrocytes. In some embodiments, expression of a polypeptide encoded by a nucleic acid comprising the nucleic acid sequence of SEQ ID NO:2, operably linked to a promoter comprising a nucleic acid comprising SEQ ID NO:7, is at a detectably greater level in a cell compared with the level of expression of a polypeptide encoded by a nucleic acid comprising the nucleic acid sequence of SEQ ID NO:2, not operably linked to a promoter comprising the nucleic acid of SEQ ID NO:7, in an otherwise identical cell. In some embodiments, a recombinant nucleic acid comprises a promoter comprising a nucleic acid sequence at least 95% identical to the nucleic acid sequence of SEQ ID NO:7 is operably linked to a nucleic acid sequence at least 95% identical to the nucleic acid sequence of SEQ ID NO:2 and induces expression of a polypeptide encoded by the nucleic acid sequence of SEQ ID NO:2 in oligodendrocytes.

A promoter may be constitutive, tissue-specific or regulated. Constitutive promoters are those which cause an operably linked gene to be expressed essentially at all times. In some embodiments, a constitutive promoter is active in most eukaryotic tissues under most physiological and developmental conditions.

Regulated promoters are those which can be activated or deactivated. Regulated promoters include inducible promoters, which are usually “off” but which may be induced to turn “on,” and “repressible” promoters, which are usually “on” but may be turned “off.” Many different regulators are known, including temperature, hormones, cytokines, heavy metals and regulatory proteins. The distinctions are not absolute; a constitutive promoter may often be regulated to some degree. In some cases, an endogenous pathway may be utilized to provide regulation of the transgene expression, e.g., using a promoter that is naturally downregulated when the pathological condition improves.

A tissue-specific promoter is a promoter that is active in only specific types of tissues, cells or organs. Typically, a tissue-specific promoter is recognized by transcriptional activator elements that are specific to a particular tissue, cell and/or organ. For example, a tissue-specific promoter may be more active in one or several particular tissues (e.g., two, three or four) than in other tissues. In some embodiments, expression of a gene modulated by a tissue-specific promoter is much higher in the tissue for which the promoter is specific than in other tissues. In some embodiments, there may be little, or substantially no activity, of the promoter in any tissue other than the one for which it is specific. A promoter may be a tissue-specific promoter, such as the mouse albumin promoter, or the transthyretin promoter (TTR), which are active in liver cells. Other examples of tissue specific promoters include promoters from genes encoding skeletal α-actin, myosin light chain 2A, dystrophin, muscle creatine kinase which induce expression in skeletal muscle (Li et al. (1999) Nat. Biotech. 17:241-245). Liver specific expression may be induced using promoters from the albumin gene (Miyatake et al. (1997) J. Virol. 71:5124-5132), hepatitis B. virus core promoter (Sandig, et al. (1996) Gene Ther. 3:1002-1009) and alpha-fetoprotein (Arbuthnot et al., (1996) Hum. Gene. Ther. 7:1503-1514).

Enhancer

In another aspect, a modified nucleic acid encoding a therapeutic polypeptide further comprises an enhancer to increase expression of the therapeutic polypeptide (e.g., a ASPA protein). Typically, an enhancer element is located upstream of a promoter element but may also be located downstream or within another sequence (e.g., a transgene). An enhancer may be located 100 nucleotides, 200 nucleotides, 300 nucleotides or more upstream or downstream of a modified nucleic acid. An enhancer typically increases expression of a modified nucleic acid (e.g., encoding a therapeutic polypeptide, e.g., encoding ASPA) beyond the increased expression provided by a promoter element alone.

Many enhancers are known in the art, including, but not limited to, the cytomegalovirus major immediate-early enhancer. More specifically, the cytomegalovirus (CMV) MIE promoter comprises three regions: the modulator, the unique region and the enhancer (Isomura and Stinski (2003) J. Virol. 77(6):3602-3614). The CMV enhancer region can be combined with another promoter, or a portion thereof, to form a hybrid promoter to further increase expression of a nucleic acid operably linked thereto. For example, a chicken β-actin (CBA) promoter, or a portion thereof, can be combined with a CMV promoter/enhancer, or a portion thereof, to make a version of CBA termed the “CBh” promoter, which stands for chicken beta-actin hybrid promoter, as described in Gray et al. (2011, Human Gene Therapy 22:1143-1153). Like promoters, enhancers may be constitutive, tissue-specific or regulated.

In some embodiments of the present disclosure, an enhancer sequence (e.g., a CMV enhancer) is operably linked to a modified nucleic acid encoding ASPA. In some embodiments, an enhancer comprising or consisting of the nucleic acid sequence of SEQ ID NO:6 or SEQ ID NO:17 (e.g., a CMV enhancer) is operably linked to a modified nucleic acid encoding ASPA. In some embodiments, an enhancer comprising a nucleic acid sequence at least 80%, at least 85%, at least 90%, at least 95%, at least 98%, at least 99% or 100% identical to the nucleic acid sequence of SEQ ID NO:6 or SEQ ID NO:17 is operably linked to a nucleic acid comprising the nucleic acid sequence of SEQ ID NO:2, and optionally operably linked to a promoter comprising the nucleic acid sequence of SEQ ID NO:7. In some embodiments, an enhancer comprising a nucleic acid sequence at least 95% identical to the nucleic acid sequence of SEQ ID NO:6 or SEQ ID NO:17 is operably linked to a nucleic acid sequence at least 95% identical to the nucleic acid sequence of SEQ ID NO:2 and induces expression of a polypeptide encoded by the nucleic acid sequence of SEQ ID NO:2 in oligodendrocytes. In some embodiments, an enhancer comprising a nucleic acid sequence at least 95% identical to the nucleic acid sequence of SEQ ID NO:6 or SEQ ID NO:17 is operably linked to a nucleic acid sequence at least 95% identical to the nucleic acid sequence of SEQ ID NO:7, and is operably linked to a nucleic acid sequence at least 95% identical to the nucleic acid sequence of SEQ ID NO:2, and together the nucleic acid sequences of SEQ ID NO:6 (or SEQ ID NO:17) and SEQ ID NO:7 induce expression of a polypeptide encoded by the nucleic acid sequence of SEQ ID NO:2 in oligodendrocytes. In some embodiments, expression of a polypeptide encoded by the nucleic acid sequence of SEQ ID NO:2, operably linked to an enhancer comprising the nucleic acid sequence of SEQ ID NO:6 (or SEQ ID NO:17), is at a detectably greater level in a cell compared with the level of expression of a polypeptide encoded by SEQ ID NO:2, not operably linked to an enhancer comprising the nucleic acid of SEQ ID NO:5, in an otherwise identical cell. In some embodiments, a recombinant nucleic acid comprises an enhancer comprising a nucleic acid sequence at least 95% identical to the nucleic acid sequence of SEQ ID NO:6 or SEQ ID NO:17, operably linked to a nucleic acid sequence at least 95% identical to the nucleic acid sequence of SEQ ID NO:7, and operably linked to a nucleic acid sequence at least 95% identical to the nucleic acid sequence of SEQ ID NO:2, and together the nucleic acid sequences of SEQ ID NO:6 (or SEQ ID NO:17) and SEQ ID NO:7 induce expression of a polypeptide encoded by the nucleic acid sequence of SEQ ID NO:2 in oligodendrocytes.

Fillers, Spacers and Stuffers

As disclosed herein, a recombinant nucleic acid intended for use in an rAAV vector may include an additional nucleic acid element to adjust the length of the nucleic acid to near, or at the normal size (e.g., approximately 4.7 to 4.9 kilobases), of the viral genomic sequence acceptable for AAV packaging into an rAAV vector (Grieger and Samulski (2005) J. Virol. 79(15):9933-9944). Such a sequence may be referred to interchangeably as filler, spacer or stuffer. In some embodiments, filler DNA is an untranslated (non-protein coding) segment of nucleic acid. In some embodiments, a filler or stuffer polynucleotide sequence is a sequence between about 1-10, 10-20, 20-30, 30-40, 40-50, 50-60, 60-70, 70-80, 80-90-90-100, 100-150, 150-200, 200-250, 250-300, 300-400, 400-500, 500-750, 750-1000, 1000-1500, 1500-2000, 2000-3000 or more in length.

AAV vectors typically accept inserts of DNA having a size ranging from about 4 kb to about 5.2 kb or about 4.1 to 4.9 kb for optimal packaging of the nucleic acid into the AAV capsid. In some embodiments, an rAAV vector comprises a vector genome having a total length between about 3.0 kb to about 3.5 kb, about 3.5 kb to about 4.0 kb, about 4.0 kb to about 4.5 kb, about 4.5 kb to about 5.0 kb or about 5.0 kb to about 5.2 kb. In some embodiments, an rAAV vector comprises a vector genome having a total length of about 4.7 kb. In some embodiments, an rAAV vector comprises a vector genome that is self-complementary. While the total length of a self-complementary (sc) vector genome in an rAAV vector is equivalent to a single-stranded (ss) vector genome (i.e., from about 4 kb to about 5.2 kb), the nucleic acid sequence (i.e., comprising the transgene, regulatory elements and ITRs) encoding the sc vector genome must be only half as long as a nucleic acid sequence encoding a ss vector genome in order for the sc vector genome to be packaged in the capsid.

Introns and Exons

In some embodiments, a recombinant nucleic acid includes, for example, an intron, exon and/or a portion thereof. An intron may function as a filler or stuffer polynucleotide sequence to achieve an appropriate length for vector genome packaging into an rAAV vector. An intron and/or an exon sequence can also enhance expression of a polypeptide (e.g., a transgene) as compared to expression in the absence of the intron and/or exon element (Kurachi et al. (1995) J. Biol. Chem. 270 (10):576-5281; WO 2017/074526). Furthermore, filler/stuffer polynucleotide sequences (also referred to as “insulators”) are well known in the art and include, but are not limited to, those described in WO 2014/144486 and WO 2017/074526.

An intron element may be derived from the same gene as a heterologous polynucleotide, or derived from a completely different gene or other DNA sequence (e.g., chicken β-actin gene, minute virus of mice (MVM)). In some embodiments, a recombinant nucleic acid includes at least one element selected from an intron and an exon derived from a non-cognate gene (i.e., not derived from the modified nucleic acid, e.g., transgene). In some embodiments, an intron is derived from a chicken β-actin gene, for example comprising or consisting of the nucleic acid sequence of SEQ ID NO: 9. In some embodiments, an intron comprises a nucleic acid sequence about 80%, about 85%, about 90%, about 95%, about 98%, about 99% or 100% identical to the nucleic acid sequence of SEQ ID NO:9. In some embodiments, an intron is derived from a MVM, for example comprising or consisting of the nucleic acid sequence of SEQ ID NO:10. In some embodiments, an intron comprises a nucleic acid sequence about 80%, about 85%, about 90%, about 95%, about 98%, about 99% or 100% identical to the nucleic acid sequence of SEQ ID NO:10. In some embodiments, an exon is derived from a chicken β-actin gene, for example comprising or consisting of the nucleic acid sequence of SEQ ID NO:8. In some embodiments, an exon comprises a nucleic acid sequence about 80%, about 85%, about 90%, about 95%, about 98%, about 99% or 100% identical to the nucleic acid sequence of SEQ ID NO:8. In some embodiments, a recombinant nucleic acid is comprised of at least one of: an enhancer sequence (e.g., SEQ ID NO:6 or SEQ ID NO:17), a promoter sequence (e.g., SEQ ID NO:7), an exon (e.g., SEQ ID NO:8 or SEQ ID NO:18) and an intron (e.g., SEQ ID NO:9, SEQ ID NO:10) and modulates expression of a heterologous polypeptide, optionally encoded by the nucleic acid sequence of SEQ ID NO:2. In some embodiments, expression of a polypeptide encoded by the nucleic acid sequence of SEQ ID NO:2, operably linked to a regulatory region comprising at least one of: an enhancer sequence (e.g., SEQ ID NO:6 or SEQ ID NO:17), a promoter sequence (e.g., SEQ ID NO:7), an exon (e.g., SEQ ID NO:8 or SEQ ID NO:18) and an intron (e.g., SEQ ID NO:9, SEQ ID NO:10), is at a detectably greater level in a cell compared with the level of expression of a polypeptide encoded by the nucleic acid sequence of SEQ ID NO:2, not operably linked to such regulatory elements in an otherwise identical cell.

In some embodiments, a recombinant nucleic acid comprises a modified nucleic acid of SEQ ID NO:2, operably linked to a regulatory element comprising at least one of: an enhancer sequence (e.g., SEQ ID NO:6 or SEQ ID NO:17), a promoter sequence (e.g., SEQ ID NO:7), an exon (e.g., SEQ ID NO:8 or SEQ ID NO:18) and an intron (e.g., SEQ ID NO:9, SEQ ID NO:10).

Polyadenylation Signal Sequence (polyA)

Further regulatory elements may include a stop codon, a termination sequence, and a polyadenylation (polyA) signal sequence, such as, but not limited to a bovine growth hormone poly A signal sequence (BHG polyA). A polyA signal sequence drives efficient addition of a poly-adenosine “tail” at the 3′ end of a eukaryotic mRNA which guides termination of gene transcription (see, e.g., Goodwin and Rottman J. Biol. Chem. (1992) 267(23):16330-16334). A polyA signal acts as a signal for the endonucleolytic cleavage of the newly formed precursor mRNA at its 3′ end and for addition to this 3′ end of an RNA stretch consisting only of adenine bases. A polyA tail is important for the nuclear export, translation and stability of mRNA. In some embodiments, a poly A is a SV40 early polyadenylation signal, a SV40 late polyadenylation signal, an HSV thymidine kinase polyadenylation signal, a protamine gene polyadenylation signal, an adenovirus 5 E1b polyadenylation signal, a growth hormone polyadenylation signal, a PBGD polyadenylation signal or an in silico designed polyadenylation signal.

In some embodiments, and optionally in combination with one or more other regulatory elements described herein, a polyA signal sequence of a recombinant nucleic acid is a polyA signal that is capable of directing and effecting the endonucleolytic cleavage and polyadenylation of the precursor mRNA resulting from the transcription of a modified nucleic acid encoding ASPA (e.g., SEQ ID NO:2). In some embodiments, a polyA sequence comprises or consists of the nucleic acid sequence of SEQ ID NO:11. In some embodiments, a polyA sequence comprises a nucleic acid sequence about 80%, about 85%, about 90%, about 95%, about 98%, about 99% or 100% identical to the nucleic acid sequence of SEQ ID NO:11. In some embodiments, a recombinant nucleic acid comprises at least one of: an enhancer sequence (e.g., SEQ ID NO:6 or SEQ ID NO:17), a promoter sequence (e.g., SEQ ID NO:7), an exon (e.g., SEQ ID NO:8 or SEQ ID NO:18), an intron (e.g., SEQ ID NO:9, SEQ ID NO:10) and a polyA (SEQ ID NO:11) and modulates the expression of a heterologous polypeptide, optionally encoded by the nucleic acid sequence of SEQ ID NO:2.

In some embodiments, an rAAV vector (e.g., AAV/Olig001-ASPA), with tropism for oligodendrocytes, contains a self-complementary vector genome comprising AAV ITRs (e.g., AAV2 ITRs) and a recombinant nucleic acid comprising a modified (i.e., codon-optimized) nucleic acid encoding ASPA and at least one of the following regulatory elements: an enhancer (e.g., a CMV enhancer), a promoter (e.g., a CBh promoter), an exon (e.g., a CBA exon 1), an intron (e.g., CBA intron, MVM intron) and a poly A (e.g., a BHG polyA).

In some embodiments, an rAAV vector (e.g., AAV/Olig001-ASPA), with tropism for oligodendrocytes, contains a self-complementary genome comprising AAV ITRs (e.g., SEQ ID NO:5, SEQ ID NO:12 and/or SEQ ID NO:19) and a recombinant nucleic acid comprising a modified (i.e., codon-optimized) nucleic acid (e.g., SEQ ID NO:2) encoding ASPA and at least one of the following regulatory elements: an enhancer (e.g., SEQ ID NO:6 or SEQ ID NO:17), a promoter (e.g., SEQ ID NO:7), an exon (e.g., a CBA exon SEQ ID NO:8 or SEQ ID NO:18), an intron (e.g., SEQ ID NO:9 and SEQ ID NO:10) and a poly A (e.g., SEQ ID NO:11).

In some embodiments, an rAAV vector (e.g., AAV/Olig001-ASPA), with tropism for oligodendrocytes, contains a self-complementary genome comprising SEQ ID NO:20.

Biological Activity of rAAV Vectors of the Disclosure

In some embodiments, an rAAV vector of the present disclosure (e.g., comprising an ASPA transgene) transduces a target cell (e.g., an oligodendrocyte) and mediates a biological activity. In some embodiments, an rAAV vector (e.g., AAV/Olig001-ASPA) transduces a target cell (e.g., an oligodendrocyte) and mediates at least one detectable activity selected from the group consisting of:

(i) reduces NAA levels in cells in vitro;

(ii) improves, increases and/or enhances balance, grip strength and/or motor coordination;

(iii) improves, increases and/or enhances latency to fall (seconds);

(iv) improves, increases and/or enhances generalized motor function;

(v) reduces, inhibits and/or neutralizes accumulation of NAA levels in vivo;

(vi) reduces, inhibits and/or neutralizes vacuole volume fraction in the thalamus;

(vii) reduces, inhibits and/or neutralizes vacuole volume fraction in the cerebellar white matter/pons;

(viii) improves, increases and/or enhances the number of oligodendrocytes in the thalamus;

(ix) improves, increases and/or enhances the number of oligodendrocytes in the cortex;

(x) improves, increases and/or enhances the number of neurons in the thalamus;

(xi) improves, increases and/or enhances the number of neurons in the cortex; and

(xii) improves, increases and/or cortical myelination.

In some embodiments, an rAAV vector which transduces a target cell (e.g., an oligodendrocyte) and mediates at least one detectable activity of (i) through (xii) is AAV/Oligo001-ASPA.

In some embodiments, a cell transduced with an rAAV vector (e.g., AAV/Olig001-ASPA) has a reduced level of NAA as compared to a level of NAA in an otherwise identical cell transduced with an rAAV comprising a wild-type nucleic acid sequence encoding ASPA (e.g., SEQ ID NO:3). In some embodiments, a cell transduced with an rAAV vector (e.g., AAV/Olig001-ASPA) has a reduced level of NAA as compared to a level of NAA in an otherwise identical cell transduced with an rAAV comprising an alternative codon-optimized nucleic acid encoding ASPA (e.g., SEQ ID NO:1). In some embodiments, a cell transduced with an rAAV vector (e.g., AAV/Olig001-ASPA) has a reduced level of NAA as compared to a level of NAA in an otherwise cell comprising a mutant nucleic acid encoding ASPA that was not transduced.

In some embodiments, a cell transduced in vivo with an rAAV vector (e.g., AAV/Olig001-ASPA) has a reduced level of NAA as compared to a level of NAA in an otherwise identical cell transduced in vivo with an rAAV comprising a wild-type nucleic acid encoding ASPA (e.g., SEQ ID NO:3). In some embodiments, a cell transduced in vivo with an rAAV vector (e.g., AAV/Olig001-ASPA) has a reduced level of NAA as compared to a level of NAA in an otherwise identical cell transduced in vivo with an rAAV comprising an alternative codon-optimized nucleic acid encoding ASPA (e.g., SEQ ID NO:1). In some embodiments, a cell transduced in vivo with an rAAV vector (e.g., AAV/Olig001-ASPA) has a reduced level of NAA as compared to a level of NAA in an otherwise identical cell comprising a mutant ASPA gene that was not transduced.

In some embodiments, balance, grip strength and/or motor coordination in a subject with an ASPA gene mutation to whom an rAAV vector (e.g., AAV/Olig001-ASPA) has been administered is significantly improved as compared to balance, grip strength and/or motor coordination of an otherwise similar subject with an ASPA gene mutation to whom the rAAV vector has not been administered, or compared to the same subject prior to administration of the rAAV vector, as measured by, e.g., rotarod performance.

In some embodiments, balance, grip strength and/or motor coordination in a subject with an ASPA gene mutation to whom an rAAV vector (e.g., AAV/Olig001-ASPA) has been administered is indistinguishable from balance, grip strength and/or motor coordination in an otherwise similar subject without an ASPA gene mutation, and to whom the rAAV vector has not been administered, as measured by, e.g., rotarod performance. In some embodiments, an rAAV vector (e.g., AAV/Olig001-ASPA) is administered via an intracerebroventricular (ICV) route of administration. In some embodiments, rotarod performance is measured as latency to fall in seconds.

In some embodiments, generalized motor function of a subject with an ASPA gene mutation to whom an rAAV vector (e.g., AAV/Olig001-ASPA) has been administered is significantly improved as compared to generalized motor function of an otherwise similar subject with an ASPA gene mutation to whom the rAAV vector is not administered, or compared to the function in the subject prior to administration of the rAAV vector, as measured by, e.g., open field activity. In some embodiments, an rAAV vector (e.g., AAV/Olig001-ASPA) is administered via an intracerebroventricular (ICV) route of administration.

In some embodiments, generalized motor function in a subject with an ASPA gene mutation to whom an rAAV vector (e.g., AAV/Olig001-ASPA) is administered is indistinguishable from generalized motor function in an otherwise similar subject without an ASPA gene mutation, and to whom the rAAV has not been administered, as measured by, e.g., open field activity. In some embodiments, an rAAV vector (e.g., AAV/Olig001-ASPA) is administered via an intracerebroventricular (ICV) route of administration.

In some embodiments, an NAA level in the brain of subject with an ASPA gene mutation to whom an rAAV vector (e.g., AAV/Olig001-ASPA) is administered is significantly reduced as compared to a NAA level in the brain of an otherwise similar subject with an ASPA gene mutation to whom the rAAV vector is not administered, or as comparted with the NAA level in the subject prior to administration of the rAAV vector. In some embodiments, an NAA level in the brain of a subject with an ASPA gene mutation to whom an rAAV vector (e.g., AAV/Olig001-ASPA) is administered is reduced or indistinguishable as compared to an NAA level in the brain of an otherwise similar subject without a ASPA gene mutation, and to whom the rAAV vector has not been administered.

In some embodiments, vacuole volume fraction in the thalamus of a subject with an ASPA gene mutation to whom an rAAV vector (e.g., AAV/Olig001-ASPA) is administered is significantly reduced as compared to vacuole fraction in the thalamus of an otherwise similar subject with an ASPA gene mutation to whom the rAAV vector is not administered, or compared with the subject prior to administration of the rAAV vector, wherein the vacuole fraction is measured by, e.g., unbiased stereology. In some embodiments, vacuole volume fraction in the cerebellar white matter/pons of a subject with an ASPA gene mutation to whom an rAAV vector (e.g., AAV/Olig001-ASPA) is administered is significantly reduced as compared to vacuole fraction in the cerebellar white matter/pons of an otherwise similar subject with an ASPA gene mutation to whom the rAAV vector is not administered, or compared with the subject prior to administration of the rAAV vector, wherein the vacuole fraction is measured by, e.g., unbiased stereology.

In some embodiments, the number of oligodendrocytes in the thalamus of a subject with an ASPA gene mutation to whom an rAAV vector (e.g., AAV/Olig001-ASPA) is administered is significantly increased as compared to the number of oligodendrocytes in the thalamus of an otherwise similar subject with an ASPA gene mutation to whom the vector is not administered, or compared with the subject before the vector is administered, wherein the number of oligodendrocytes in the thalamus is measured by, e.g., IHC using Olig2 antibody and unbiased stereology. In some embodiments, the number of oligodendrocytes in the brain cortex of a subject with an ASPA gene mutation to whom an rAAV vector (e.g., AAV/Olig001-ASPA) is administered is significantly increased as compared to the number of oligodendrocytes in the brain cortex of an otherwise similar subject with an ASPA gene mutation to whom the rAAV vector is not administered, or compared with the same subject before the vector is administered, wherein the number of oligodendrocytes in the brain cortex is measured by, e.g., IHC using Olig2 antibody and unbiased stereology. In some embodiments, the number of oligodendrocytes in the brain cortex of a subject with an ASPA gene mutation to whom an rAAV vector (e.g., AAV/Olig001-ASPA) is administered is indistinguishable from the number of oligodendrocytes in the brain cortex of an otherwise similar subject without a ASPA gene mutation, and to whom the rAAV vector is not administered, wherein the number of oligodendrocytes in the brain cortex is measured by, e.g., IHC using Olig2 antibody and unbiased stereology.

In some embodiments, the number of neurons in the thalamus of a subject with an ASPA gene mutation to whom an rAAV vector (e.g., AAV/Olig001-ASPA) is administered is significantly increased as compared to the number of neurons in the thalamus of an otherwise identical subject with an ASPA gene mutation to whom the rAAV vector is not administered, or compared with the number of neurons in the thalamus of the subject prior to administration of the vector, wherein the number of neurons in the thalamus is measured by, e.g., IHC using NeuN antibody and unbiased stereology. In some embodiments, the number of neurons in the brain cortex of a subject with an ASPA gene mutation to whom an rAAV vector (e.g., AAV/Olig001-ASPA) is administered is significantly increased as compared to the number of neurons in the brain cortex of an otherwise similar subject with an ASPA gene mutation to whom the rAAV vector is not administered, or as compared with the number of neurons in the brain cortex of the subject prior to administration of the vector, wherein the number of neurons in the brain cortex is measured by, e.g., IHC using NeuN antibody and unbiased stereology. In some embodiments, the number of neurons in the brain cortex of a subject with an ASPA gene mutation to whom an rAAV vector (e.g., AAV/Olig001-ASPA) is administered is indistinguishable from the number of neurons in the brain cortex of an otherwise similar subject without an ASPA gene mutation, and to whom the rAAV vector is not administered, wherein the number of neurons in the brain cortex is measured by, e.g., IHC using NeuN antibody and unbiased stereology.

In some embodiments, cortical myelination in the brain of a subject with an ASPA gene mutation to whom an rAAV vector (e.g., AAV/Oligo001-ASPA) is administered is significantly increased as compared to cortical myelination in the brain of an otherwise similar subject with an ASPA gene mutation to whom the rAAV vector is not administered, or compared with the cortical myelination in the brain of the subject prior to administration of the vector, wherein the cortical myelination is measured by, e.g., cortical myelin basic protein-positive fiber length density (MBP-LD).

### Assembly of Viral Vectors

A viral vector (e.g., rAAV vector) carrying a transgene (e.g., ASPA) is assembled from a polynucleotide encoding a transgene, suitable regulatory elements and elements necessary for production of viral proteins which mediate cell transduction. Examples of a viral vector include but are not limited to adenoviral, retroviral, lentiviral, herpesvirus and adeno-associated virus (AAV) vectors, and in particular rAAV vector (as discussed, supra).

A vector genome component of an rAAV vector produced according to the methods of the disclosure include at least one transgene, e.g., a modified nucleic acid encoding ASPA and associated expression control sequences for controlling expression of the modified nucleic acid encoding ASPA.

In a preferred embodiment, a vector genome includes a portion of a parvovirus genome, such as an AAV genome with rep and cap deleted and/or replaced by a modified nucleic acid (e.g., transgene, e.g., modified nucleic acid encoding ASPA) and its associated expression control sequences. A modified nucleic acid encoding ASPA is typically inserted adjacent to one or two (i.e., is flanked by) AAV ITRs or ITR elements adequate for viral replication (Xiao et al. (1997) J. Virol. 71(2): 941-948), in place of the nucleic acid encoding viral rep and cap proteins. Other regulatory sequences suitable for use in facilitating tissue-specific expression of a modified nucleic acid encoding ASPA in the target cell (e.g., oligodendrocyte) may also be included.

Packaging Cell

One skilled in the art would appreciate that an rAAV vector comprising a transgene, and lacking virus proteins needed for viral replication (e.g., cap and rep), cannot replicate since such proteins are necessary for virus replication and packaging. Cap and rep genes may be supplied to a cell (e.g., a host cell, e.g., a packaging cell) as part of a plasmid that is separate from a plasmid supplying the vector genome with the transgene.

“Packaging cell” or “producer cell” means a cell or cell line which may be transfected with a vector, plasmid or DNA construct, and provides in trans all the missing functions which are required for the complete replication and packaging of a viral vector. The required genes for rAAV vector assembly include the vector genome (e.g., a modified nucleic acid encoding ASPA, regulatory elements, and ITRs), AAV rep gene, AAV cap gene, and certain helper genes from other viruses such as, e.g., adenovirus. One of ordinary skill would understand that the requisite genes for AAV production can be introduced into a packaging cell in various ways including, for example, transfection of one or more plasmids. However, in some embodiments, some genes (e.g., rep, cap, helper) may already be present in a packaging cell, either integrated into the genome or carried on an episome. In some embodiments, a packaging cell expresses, in a constitutive or inducible manner, one or more missing viral functions.

Any suitable packaging cell known in the art may be employed in the production of a packaged viral vector. Mammalian cells or insect cells are preferred. Examples of cells useful for the production of a packaging cell in the practice of the disclosure include, for example, human cell lines, such as PER.C6, WI38, MRCS, A549, HEK293 cells (which express functional adenoviral E1 under the control of a constitutive promoter), B-50 or any other HeLa cell, HepG2, Saos-2, HuH7, and HT1080 cell lines. Suitable non-human mammalian cell lines include, for example, VERO, COS-1, COS-7, MDCK, BHK21-F, HKCC or CHO cells.

In some embodiments, a packaging cell is capable of growing in suspension culture. In some embodiments, a packaging cell is capable of growing in serum-free media. For example, HEK293 cells are grow in suspension in serum free medium. In another embodiment, a packaging cell is a HEK293 cell as described in U.S. Pat. No. 9,441,206 and deposited as American Type Culture Collection (ATCC) No. PTA 13274. Numerous rAAV packaging cell lines are known in the art, including, but not limited to, those disclosed in WO 2002/46359.

A cell line for use as a packaging cell includes insect cell lines. Any insect cell which allows for replication of AAV and which can be maintained in culture can be used in accordance with the present disclosure. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, N J (1995); O'Reilly et al., Baculovirus Expression Vectors: A Laboratory Manual, Oxford Univ. Press (1994); Samulski et al. (1989) J. Virol. 63:3822-3828; Kajigaya et al. (1991) Proc. Nat'l. Acad. Sci. USA 88: 4646-4650; Ruffing et al. (1992) J. Virol. 66:6922-6930; Kimbauer et al. (1996) Virol. 219:37-44; Zhao et al. (2000) Virol. 272:382-393; and U.S. Pat. No. 6,204,059.

As a further alternative, viral vectors of the disclosure may be produced in insect cells using baculovirus vectors to deliver the rep/cap genes and rAAV template as described, for example, by Urabe et al. (2002) Human Gene Therapy 13:1935-1943. When using baculovirus production for AAV, in some embodiments, a vector genome is self-complementary. In some embodiments, a host cell is a baculovirus-infected cell (e.g., an insect cell) comprising, optionally, additional nucleic acids encoding baculovirus helper functions, thereby facilitating production of a viral capsid.

A packaging cell generally includes one or more viral vector functions along with helper functions and packaging functions sufficient to result in replication and packaging of the viral vector. These various functions may be supplied together, or separately, to the packaging cell using a genetic construct such as a plasmid or an amplicon, and they may exist extrachromosomally within the cell line, or integrated into the host cell's chromosomes. In some embodiments, a packaging cell is transfected with at least i) a plasmid comprising a vector genome comprising a codon-optimized human ASPA transgene (e.g., SEQ ID NO:2) and AAV ITRs (e.g., SEQ ID NO:5 and SEQ ID NO:12) and further comprising at least one of the following regulatory elements: an enhancer (e.g., SEQ ID NO:6), a promoter (e.g., SEQ ID NO:7), an exon (e.g., a CBA exon SEQ ID NO:8), an intron (e.g., SEQ ID NO:9 and SEQ ID NO:10) and a poly A (e.g., SEQ ID NO:11) and ii) a plasmid comprising a rep gene (e.g., AAV2 rep) and a cap gene (e.g., Olig001 cap).

In some embodiments, a host cell is supplied with one or more of the packaging or helper functions incorporated within, e.g., a host cell line with one or more vector functions incorporated extrachromosomally or integrated into the cell's chromosomal DNA.

Helper Function

AAV is a Dependovirus in that it cannot replicate in a cell without co-infection of the cell by a helper virus. Helper functions include helper virus elements needed for establishing active infection of a packaging cell, which is required to initiate packaging of the viral vector. Helper viruses include, typically, adenovirus or herpes simplex virus. Adenovirus helper functions typically include adenovirus components adenovirus early region 1A (Ela), E1b, E2a, E4, and viral associated (VA) RNA. Helper functions (e.g., E1a, E1b, E2a, E4, and VA RNA) can be provided to a packaging cell by transfecting the cell with one or more nucleic acids encoding various helper elements. Alternatively, a host cell (e.g., a packaging cell) can comprise a nucleic acid encoding the helper protein. For instance, HEK293 cells were generated by transforming human cells with adenovirus 5 DNA and now express a number of adenoviral genes, including, but not limited to E1 and E3 (see, e.g., Graham et al. (1977) J. Gen. Virol. 36:59-72). Thus, those helper functions can be provided by the HEK 293 packaging cell without the need of supplying them to the cell by, e.g., a plasmid encoding them. In some embodiments, a packaging cell is transfected with at least i) a plasmid comprising a vector genome comprising a codon-optimized human ASPA transgene (e.g., SEQ ID NO:2) and AAV ITRs (e.g., SEQ ID NO:5 and SEQ ID NO:12) and further comprising at least one of the following regulatory elements: an enhancer (e.g., SEQ ID NO:6), a promoter (e.g., SEQ ID NO:7), an exon (e.g., a CBA exon SEQ ID NO:8), an intron (e.g., SEQ ID NO:9 and SEQ ID NO:10) and a poly A (e.g., SEQ ID NO:11), ii) a plasmid comprising a rep gene (e.g., AAV2 rep) and a cap gene (e.g., Olig001 cap) and iii) a plasmid comprising a helper function.

Any method of introducing a nucleotide sequence carrying a helper function into a cellular host for replication and packaging may be employed, including but not limited to, electroporation, calcium phosphate precipitation, microinjection, cationic or anionic liposomes, and liposomes in combination with a nuclear localization signal. In some embodiments, helper functions are provided by transfection using a virus vector, or by infection using a helper virus, standard methods for producing viral infection may be used.

The vector genome may be any suitable recombinant nucleic acid, such as a DNA or RNA construct and may be single stranded, double stranded, or duplexed (i.e., self complementary as described in WO 2001/92551).

### Production of Packaged Viral Vector

Viral vectors can be made by several methods known to skilled artisans (see, e.g., WO 2013/063379). A preferred method is described in Grieger, et al. (2015) Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using a triple transfection method (e.g., WO 96/40240), a HEK293 cell line suspension can generate greater than 1×10⁵ vector genome containing particles (vg)/cell, or greater than 1×10¹⁴ vg/L of cell culture, when harvested 48 hours post-transfection. More specifically, triple transfection refers a method whereby a packaging cell is transfected with three plasmids: one plasmid encodes the AAV rep and cap (e.g., Olig001 cap) genes, another plasmid encodes various helper functions (e.g., adenovirus or HSV proteins such as E1a, E1b, E2a, E4, and VA RNA, and another plasmid encodes a transgene (e.g., ASPA) and various elements to control expression of the transgene.

Single-stranded vector genomes are packaged into capsids as the plus strand or minus strand in about equal proportions. In some embodiments of an rAAV vector, a vector genome is in the plus strand polarity (i.e., the sense or coding sequence of the DNA strand). In some embodiments an rAAV vector, a vector is in the minus strand polarity (i.e., the antisense or template DNA strand). Given the nucleotide sequence of a plus strand in its 5′ to 3′ orientation, the nucleotide sequence of a minus strand in its 5′ to 3′ orientation can be determined as the reverse-complement of the nucleotide sequence of the plus strand.

To achieve the desired yields, a number of variables are optimized such as selection of a compatible serum-free suspension media that supports both growth and transfection, selection of a transfection reagent, transfection conditions and cell density.

An rAAV vector may be purified by methods standard in the art such as by column chromatography or cesium chloride gradients. Methods for purifying rAAV vectors are known in the art and include methods described in Clark et al. (1999) Human Gene Therapy 10(6):1031-1039; Schenpp and Clark (2002) Methods Mol. Med. 69:427-443; U.S. Pat. No. 6,566,118 and WO 98/09657

A universal purification strategy, based on ion exchange chromatography methods, may be used to generate high purity vector preps of AAV serotypes 1-6, 8, 9 and various chimeric capsids (e.g., Olig001). In some embodiment, this process can be completed within one week, result in high full to empty capsid ratios (>90% full capsids), provide post-purification yields (>1×10¹³ vg/L) and purity suitable for clinical applications. In some embodiments, such a method is universal with respect to all serotypes and chimeric capsids. Scalable manufacturing technology may be utilized to manufacture GMP clinical and commercial grade rAAV vectors (e.g., for the treatment of Canavan disease).

After rAAV vectors of the present disclosure have been produced and purified, they can be titered (e.g., the amount of rAAV vector in a sample can be quantified) to prepare compositions for administration to subjects, such as human subjects with Canavan disease. rAAV vector titering can be accomplished using methods know in the art.

In some embodiments, the number of viral particles, including particles containing a vector genome and “empty” capsids that do not contain a vector genome, can be determined by electron microscopy, e.g., transmission electron microscopy (TEM). Such a TEM-based method can provide the number of vector particles (or virus particles in the case of wild type AAV) in a sample.

In some embodiments, rAAV vector genomes can be titered using quantitative PCR (qPCR) using primers against sequences in the vector genome, for example ITR sequences (e.g, SEQ ID NO:5, SEQ ID NO:12 or SEQ ID NO:19), and/or sequences in the transgene (e.g., SEQ ID NO:2) or regulatory elements. By performing qPCR in parallel on dilutions of a standard of known concentration, such as a plasmid containing the sequence of the vector genome, a standard curve can be generated permitting the concentration of the rAAV vector to be calculated as the number of vector genomes (vg) per unit volume such as microliters or milliliters. By comparing the number of vector particles as measured by, e.g., electron microscopy, to the number of vector genomes in a sample, the number of empty capsids can be determined. Because the vector genome contains the therapeutic transgene, vg/kg or vg/ml of a vector sample may be more indicative of the therapeutic amount of the vector that a subject will receive than the number of vector particles, some of which may be empty and not contain a vector genome. Once the concentration of rAAV vector genomes in the stock solution is determined, it can be diluted into or dialyzed against suitable buffers for use in preparing a composition for administration to subjects (e.g., subjects with Canavan disease).

### Methods of Treatment

A modified nucleic acid, such as a modified nucleic acid encoding ASPA, as disclosed herein, may be used for gene therapy treatment and/or prevention of a disease, disorder or condition associated with deficiency or dysfunction of an ASPA polypeptide (e.g., Canavan disease), and of any other condition and or illness in which an upregulation of an ASPA gene may produce a therapeutic benefit or improvement, e.g., a disease, disorder or condition mediated by, or associated with, a decrease in the level or function of an ASPA polypeptide compared with the level or function of an ASPA polypeptide in an otherwise healthy individual.

A vector genome and/or an rAAV vector comprising a modified nucleic acid encoding ASPA, as disclosed, herein may be used for gene therapy treatment and/or prevention of a disease, disorder or condition associated with or caused by deficiency or dysfunction of an ASPA enzyme (e.g., Canavan disease), and of any other condition and/or illness in which an upregulation of an ASPA enzyme may produce a therapeutic benefit or improvement. In some embodiments, methods of the disclosure include use of an rAAV vector, or a pharmaceutical composition thereof, in the treatment of Canavan disease in a subject. In some embodiments, methods of the disclosure include use of an rAAV vector (e.g., AAV/Oligo001-ASPA), or pharmaceutical composition thereof, to increase the level of ASPA in a subject in need thereof.

A modified nucleic encoding ASPA, a vector genome comprising a modified nucleic acid encoding ASPA and/or an rAAV vector (e.g., AAV/Oligo001-ASPA) comprising a modified nucleic acid encoding ASPA of the disclosure, may be used in the preparation of a medicament for use in the treatment and/or prevention of a disease, disorder or condition associated with or caused by deficiency or dysfunction of ASPA (e.g., a decreased level of functional ASPA enzyme such as in Canavan disease) and of any other condition or illness in which an upregulation of ASPA may produce a therapeutic benefit or improvement.

In some embodiments, gene therapy treatment and/or prevention of a disease, disorder or condition associated with deficiency or dysfunction of an ASPA enzyme (e.g., Canavan disease), and of any other condition and/or illness in which an upregulation of ASPA gene expression, and/or increased expression of a functional ASPA enzyme, may produce a therapeutic benefit or improvement, comprises administration of a therapeutically effective amount of a modified nucleic acid encoding ASPA, a vector genome comprising a modified nucleic acid encoding ASPA and/or an rAAV vector (e.g., AAV/Oligo001-ASPA) comprising a modified nucleic acid encoding ASPA of the disclosure to a subject (e.g., a patient) in need of treatment.

Treatment of a subject (e.g., a patient) with a therapeutically effective amount of a modified nucleic acid encoding ASPA, a vector genome comprising a modified nucleic acid encoding ASPA and/or an rAAV vector (e.g., AAV/Oligo001-ASPA) comprising a modified nucleic acid ASPA of the disclosure may alleviate, ameliorate, treat, prevent or reduce the severity of one or more symptoms of Canavan disease as compared to a baseline measurement, such as a measurement in the same individual prior to initiation of treatment described herein, or a measurement in a control individual (or multiple control individuals thereby establishing a level for comparision) in the absence of the treatment described herein. In some embodiments, a “control individual” is an individual afflicted with the same form of disease or injury as an individual being treated, but who is not currently being treated, but may receive treatment in the future.

For example, treatment of a subject with a therapeutically effective amount of a modified nucleic acid encoding ASPA, a vector genome comprising a modified nucleic acid encoding ASPA and/or an rAAV vector (e.g., AAV/Oligo001-ASPA) may reduce NAA accumulation as compared to NAA accumulation in a control individual, or as compared to NAA accumulation in the same individual prior to treatment. In some embodiments, NAA accumulation is reduced by about 10%, about 20%, about 30%, about 40%, about 50%, about 60%, about 70%, about 80%, about 90% or by about 100% in a subject who is treated as compared to a control individual, or as compared with the same individual prior to treatment.

In some embodiments, treatment of a subject with a therapeutically effective amount of a modified nucleic acid encoding ASPA, a vector genome comprising a modified nucleic acid encoding ASPA and/or an rAAV vector (e.g., AAV/Oligo001-ASPA) may increase aspartate and/or increase acetate levels as compared to aspartate and/or acetate levels in a control individual, or as compared to aspartate and/or acetate levels in the same individual prior to treatment. In some embodiments, aspartate and/or acetate levels are increased by about 10%, about 20%, about 30%, about 40%, about 50%, about 60%, about 70%, about 80%, about 90% or by about 100% in a subject who is treated as compared to a control individual, or as compared with the same individual prior to treatment.

In some embodiments, treatment may also alleviate, ameliorate, treat, prevent or reduce the severity of degeneration of myelin in the brain and spinal cord, intellectual disability, loss of previously acquired motor skills, feeding difficulties, abnormal muscle tone, macrocephaly, paralysis and seizures and/or a delay in development of speech and motor skills as compared to the same in a control individual, or in a subject prior to treatment. In some embodiments, treatment of a subject (e.g., a patient) with a therapeutically effective amount of a modified nucleic acid encoding ASPA, a vector genome comprising a modified nucleic acid encoding ASPA and/or an rAAV vector comprising a modified nucleic acid encoding ASPA of the disclosure may also increase, improve, prevent further loss of, or enhance balance, grip, strength and or motor coordination and generalized motor function as compared to the same in a control individual, or as compared to the same subject prior to treatment. In some embodiments, treatment of a subject (e.g., a patient) with a therapeutically effective amount of a modified nucleic acid encoding ASPA, a vector genome comprising a modified nucleic acid encoding ASPA and/or an rAAV comprising a modified nucleic acid encoding ASPA of the disclosure may reduce vacuole volume fraction in the brain (e.g., thalamus, cerebellar white matter/pons), increase the number of oligodendrocytes in the brain (e.g., thalamus, cortex), increase the number of neurons in the brain (e.g., thalamus, cortex) and/or increase cortical myelination as compared to the same in a control individual, or as compared to the same subject prior to treatment.

A subject appropriate for treatment includes any subject having, or at risk of, producing an insufficient amount, or having a deficiency of, a functional gene product (protein), or that produces an aberrant, partially functional or non-function gene product (protein, e.g., an enzyme), which can lead to disease. In some embodiments, a patient is treated with a vector or pharmaceutical composition of the present disclosure prior to exhibiting any symptoms of a disease, disorder or condition (e.g., Canavan disease). In some embodiments, a patient who has been diagnosed as at-risk for a disease, disorder or condition (e.g., Canavan disease) by genetic analysis is treated with an rAAV vector or composition of the present disclosure prior to exhibiting symptoms.

In some embodiments, a subject to be treated may be mammal, and in particular a subject is a human patient, for example, a patient with Canavan disease. A subject may be in need of treatment because, as a result of one or more mutations in the coding sequence of the ASPA gene, the ASPA protein has an incorrect amino acid sequence, and thereby has decreased or no function, is expressed in the wrong tissues or at the wrong time, is under expressed or not expressed at all. A modified nucleic acid encoding ASPA of the present invention may be administered to enhance, improve or provide production of a functional ASPA enzyme which can, in turn, catalyze the breakdown of NAA to aspartate and acetate, among other biological functions as discussed elsewhere herein.

A target cell of the rAAV vector of the instant invention is a cell, in particular an oligodendrocyte, this is normally, endogenously capable of expressing the ASPA enzyme, such as those of in the brain of a mammal.

In embodiments that refer to a method of treatment as described herein, such embodiments are also further embodiments for use in that treatment, or alternatively for the manufacture of a medicament for use in that treatment.

### Pharmaceutical Compositions

In particular embodiments, the present disclosure provides a pharmaceutical composition, or medicament, for preventing or treating a disease, disorder or condition mediated by or associated with decreased expression and/or activity of ASPA, e.g., Canavan disease. In some embodiments, a pharmaceutical composition comprises a modified nucleic acid, a recombinant nucleic acid, a viral vector genome, an expression vector, a host cell or an rAAV vector, and a pharmaceutically acceptable carrier.

In some embodiments, a pharmaceutical composition comprises a therapeutically effective amount of a vector (e.g., viral vector genome, expression vector, rAAV vector) or host cell comprising a modified nucleic acid encoding ASPA which can increase the level of expression and/or the level of activity of ASPA in a cell.

In some embodiments, a pharmaceutical composition comprises a therapeutically effective amount of a vector (e.g., viral vector genome, expression vector, rAAV vector) or host cell (e.g., for ex vivo gene therapy) comprising a modified, nucleic acid encoding ASPA wherein the composition further comprises a pharmaceutically-acceptable carrier, adjuvant, diluent, excipient and/or other medicinal agents. A pharmaceutically acceptable carrier, adjuvant, diluent, excipient or other medicinal agent is one that is not biologically or otherwise undesirable, e.g., the material may be administered to a subject without causing undesirable biological effects which outweigh the advantageous biological effects of the material.

Any suitable pharmaceutically acceptable carrier or excipient can be used in the preparation of a pharmaceutical composition according to the invention (See e.g., Remington The Science and Practice of Pharmacy, Alfonso R. Gennaro (Editor) Mack Publishing Company, April 1997).

A pharmaceutical composition is typically sterile, pyrogen-free and stable under the conditions of manufacture and storage. A pharmaceutical composition may be formulated as a solution (e.g., water, saline, dextrose solution, buffered solution, or other pharmaceutically sterile fluid), microemulsion, liposome, or other ordered structure suitable to accommodate a high product (e.g., viral vector particles, microparticles or nanoparticles) concentration. In some embodiments, a pharmaceutical composition comprising a modified nucleic acid, vector genome comprising the modified nucleic acid, host cell or rAAV vector of the disclosure is formulated in water or a buffered saline solution. A carrier may be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by use of a coating such as lecithin, by maintenance of a required particle size, in the case of dispersion, and by the use of surfactants. In some embodiments, it may be preferable to include isotonic agents, for example, a sugar, a polyalcohol such as mannitol, sorbitol, or sodium chloride in the composition. Prolonged adsorption of an injectable composition can be brought about by including, in the composition, an agent which delays absorption, e.g., a monostearate salt and gelatin. In some embodiments, a nucleic acid, vector and/or host cell of the disclosure may be administered in a controlled release formulation, for example, in a composition which includes a slow release polymer or other carrier that protects the product against rapid release, including an implant and microencapsulated delivery system.

In some embodiments, a pharmaceutical composition of the disclosure is a parenteral pharmaceutical composition, including a composition suitable for intravenous, intraarterial, subcutaneous, intradermal, intraperitoneal, intramuscular, intraarticular, intraparenchymal (IP), intrathecal (IT), intracerebroventricular (ICV) and/or intracisternal magna (ICM) administration. In some embodiments, a pharmaceutical composition comprising an rAAV vector comprising a modified nucleic acid encoding ASPA is formulated for administration by ICV injection.

In some embodiments, an rAAV vector (e.g., AAV/Olig001 ASPA) is formulated in 350 mM NaCl and 5% D-sorbitol in PBS.

### Methods of Administration

A modified nucleic acid encoding a transgene (e.g., ASPA), or a vector (e.g., vector genome, rAAV vector) comprising a modified nucleic acid of the disclosure, may be administered to a subject (e.g., a patient) in order to treat the subject. Administration of a vector to a human subject, or an animal in need thereof, can be by any means known in the art for administering a vector. A target cell of a vector of the present disclosure includes cells of the CNS, preferably oligodendrocytes.

A vector can be administered in addition to, and as an adjunct to, the standard of care treatment. That is, the vector can be co-administered with another agent, compound, drug, treatment or therapeutic regimen, either simultaneously, contemporaneously, or at a determined dosing interval as would be determined by one skilled in the art using routine methods. Uses disclosed herein include administration of an rAAV vector of the disclosure at the same time, in addition to and/or on a dosing schedule concurrent with, the standard of care for Canavan disease as known in the art.

In some embodiments, a combination composition includes one or more immunosuppressive agents. In some embodiments, a combination composition includes an rAAV vector comprising a transgene (e.g., a modified nucleic acid encoding ASPA) and one or more immunosuppressive agents. In some embodiments, a method includes administering or delivering an rAAV vector comprising a transgene (e.g., a modified nucleic acid encoding ASPA) to a subject and administering an immunosuppressive agent to the subject either prophylactically prior to administration of the vector, or after administration of the vector (i.e., either before or after symptoms of a response against the vector and/or the protein provided thereby are evident).

In some embodiments, an rAAV of the invention can be co-administered with empty capsids (i.e., a virus capsid that does not contain a nucleic acid molecule or vector genome) comprising the same, or a different, capsid protein as an rAAV vector comprising a modified nucleic acid (e.g., encoding ASPA). One skilled in the art would understand that co-administration of empty capsids may decrease an immune response, e.g., a neutralizing response, to an rAAV of the disclosure. Without wishing to be bound by any particular theory, an empty capsid may serve as an immune decoy allowing an rAAV vector comprising a modified nucleic acid (e.g., encoding ASPA) to avoid a neutralizing antibody (Nab) immune response as discussed in, e.g., WO 2015/013313.

In one embodiment, a vector of the disclosure (e.g., an rAAV vector comprising a modified nucleic acid encoding ASPA) is administered systemically. Exemplary methods of systemic administration include, but are not limited to, intravenous (e.g., portal vein), intraarterial (e.g., femoral artery, hepatic artery), intravascular, subcutaneous, intradermal, intraperitoneal, transmucosal, intrapulmonary, intralymphatic and intramuscular administration, and the like, as well as direct tissue or organ injection. One skilled in the art would appreciate that systemic administration can deliver a modified nucleic acid (e.g., a modified nucleic acid encoding ASPA) to all tissues. In some embodiments, direct tissue or organ administration includes administration to the liver. In some embodiments, direct tissue or organ administration includes administration to areas directly affected by ASPA deficiency (e.g., brain and/or central nervous system). In some embodiments, vectors of the disclosure, and pharmaceutical compositions thereof, are administered to the brain parenchyma (i.e., by intraparenchymal administration), to the spinal canal or the subarachnoid space so that it reaches the cerebrospinal fluid (CSF) (i.e., by intrathecal administration), to a ventricle of the brain (i.e., by intracerebroventricular administration) and/or to the cisterna magna of the brain (i.e., by intracisternal magna administration).

Accordingly, in some embodiments, a vector of the present disclosure comprising a modified nucleic acid encoding ASPA is administered by direct injection into the brain (e.g., into the parenchyma, ventricle, cisterna magna, etc.) and/or into the CSF (e.g., into the spinal canal or subarachnoid space) to treat a neurodegenerative aspect of Canavan disease. A target cell of a vector of the present disclosure includes a cell located in the cortex, subcortical white matter of the corpus callosum, striatum and/or cerebellum. In some embodiments, a target cell of a vector of the present disclosure is an oligodendrocyte. Additional routes of administration may also comprise local application of a vector under direct visualization, e.g., superficial cortical application, or other nonstereotaxic application.

In some embodiments, a vector of the disclosure is administered by at least two routes. For example, a vector is administered systemically and also directly into the brain. If administered via at least two routes, the administration of a vector can be, but need not be, simultaneous or contemporaneous. Instead, administration via different routes can be performed separately with an interval of time between each administration.

A modified nucleic acid encoding ASPA, a vector genome comprising a modified nucleic acid encoding ASPA and/or an rAAV vector comprising a modified nucleic acid encoding ASPA of the disclosure, may be used for transduction of a cell ex vivo or for administration directly to a subject (e.g., directly to the CNS of a patient with Canavan disease). In some embodiments, a transduced cell (e.g., a host cell) is administered to a subject to treat or prevent a disease, disorder or condition (e.g., cell therapy for Canavan disease). An rAAV vector comprising a modified therapeutic nucleic acid (e.g., encoding ASPA) is preferably administered to a cell in a biologically-effective amount. In some embodiments, a biologically-effective amount of a vector is an amount that is sufficient to result in transduction and expression of a modified nucleic acid encoding ASPA (i.e., a transgene) in a target cell.

In some embodiments, the disclosure includes a method of increasing the level and/or activity of ASPA in a cell by administering to a cell (in vivo, in vitro or ex vivo) a modified nucleic acid encoding ASPA, either alone or in a vector (including a plasmid, a virus vector, a nanoparticle, a liposome, or any known method for providing a nucleic acid to a cell).

The dosage amount of an rAAV vector depends upon, e.g., the mode of administration, disease or condition to be treated, the stage and/or aggressiveness of the disease, individual subject's condition (age, sex, weight, etc.), particular viral vector, stability of protein to be expressed, host immune response to the vector, and/or gene to be delivered. Generally, doses range from at least 1×10⁸, or more, e.g., 1×10⁹, 1×10¹⁰, 1×10¹¹, 1×10¹², 1×10¹³, 1×10¹⁴, 1×10¹⁵ or more vector genomes (vg) per kilogram (kg) of body weight of the subject to achieve a therapeutic effect.

In some embodiments, a modified nucleic acid encoding ASPA may be administered as a component of a DNA molecule (e.g., a recombinant nucleic acid) having a regulatory element (e.g, a promoter) appropriate for expression in a target cell (e.g., oligodendrocytes). The modified nucleic acid encoding ASPA may be administered as a component of a plasmid or a viral vector, such as an rAAV vector. An rAAV vector may be administered in vivo by direct delivery of the vector (e.g., directly to the CNS) to a patient (e.g., a Canavan patient) in need of treatment. An rAAV vector may be administered to a patient ex vivo by administration of the vector in vitro to a cell from a donor patient in need of treatment, followed by introduction of the transduced cell back into the donor (e.g., cell therapy).

The present disclosure includes a method of administration that results in a level of mRNA encoding ASPA, a level of ASPA protein expression, and/or a level of ASPA activity that is detectably greater than the level of ASPA expression (mRNA and/or protein) or ASPA activity in an otherwise identical cell that is not administered a modified nucleic acid (e.g., a modified nucleic acid encoding ASPA).

In another embodiment, the present disclosure includes a method of administration that results in a level of mRNA encoding functional ASPA, and/or a level of functional (e.g., biologically active) ASPA protein expression, that is detectably greater than the level of functional ASPA (mRNA and/or protein) present in an otherwise identical cell that is not administered the modified nucleic acid (e.g., a modified nucleic acid encoding ASPA). That is, the present invention includes method of increasing the level of functional ASPA in a cell where the cell produces a normal level of ASPA but the ASPA protein lacks activity or demonstrates decreased activity compared with normal wild type ASPA.

A skilled artisan would understand that a cell can be cultured or grown in vitro, or can be present in an organism (i.e., in vivo). Further, a cell may express endogenous ASPA such that the level of ASPA in the cell is increased, and/or the cell expresses an endogenous ASPA that is a mutant or variant of wild type ASPA, e.g., ASPA having the sequence of SEQ ID NO:3, especially as there may be more than one wild type allele for human ASPA. Thus, the level of ASPA is increased as compared with the level of ASPA expressed in an otherwise identical, but untreated cell.

### Kits

The present disclosure provides a kit with packaging material and one or more components therein. A kit typically includes a label or packaging insert including a description of the components or instructions for use in vitro, in vivo or ex vivo, of the components therein. A kit can contain a collection of such components, e.g., a modified nucleic acid, a recombinant nucleic acid, a vector genome, an rAAV vector an rAAV, and optionally a second active agent such as a compound, therapeutic agent, drug or composition.

A kit refers to a physical structure that contains one or more components of the kit. Packaging material can maintain the components in a sterile manner and can be made of material commonly used for such purposes (e.g., paper, glass, plastic, foil, ampules, vials, tubes, etc).

A label or insert can include identifying information of one or more components therein, dose amounts, clinical pharmacology of the active ingredients(s) including mechanism of action, pharmacokinetics and pharmacodynamics. A label or insert can include information identifying manufacture, lot numbers, manufacture location and date, expiration dates. A label or insert can include information on a disease (e.g., Canavan disease) for which a kit component may be used. A label or insert can include instructions for a clinician or subject for using one or more of the kit components in a method, use or treatment protocol or therapeutic regimen. Instructions can include dosage amounts, frequency of duration and instructions for practicing any of the methods, uses, treatment protocols or prophylactic or therapeutic regimens described herein.

A label or insert can include information on potential adverse side effects, complications or reaction, such as a warning to a subject or clinician regarding situations where it would not be appropriate to use a particular composition.

### EQUIVALENTS

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, text books, and the like, and the references cited therein, to the extent that they are not already, are hereby incorporated herein by reference in their entirety.

### Exemplary Embodiments

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1: Dose-Responsive Reduction in NAA Using an rAAV Vector Comprising a Codon-Optimized Nucleic Acid Encoding ASPA

Human embryonic kidney (HEK) cells were transfected with 1.0 ug of plasmid expressing NAA synthase (Nat8L) and co-transfected with 0.1, 0.2, 0.5 or 1.0 μg of a plasmid comprising either the wild type human ASPA nucleic acid sequence (SEQ ID NO:3), a codon-optimized nucleic acid encoding ASPA (comprising the nucleic acid sequence of SEQ ID NO:1, see, Francis et al. (2016) Neurobiol. Dis. 96:323-334) or a codon-optimized nucleic acid encoding ASPA comprising the nucleic acid sequence of SEQ ID NO:2. NAA concentration was measured by HPLC (n=4/group). A dose-responsive reduction in NAA was observed in cultures transfected using the codon-optimized nucleic acid encoding ASPA of SEQ ID NO:2 relative to the cultures transfected with either the wild-type nucleic acid encoding ASPA or the codon-optimized nucleic acid encoding ASPA of SEQ ID NO:1 (FIG. 1).

### Example 2: Biodistribution of an Oligotropic AAV/Olig001

This study was undertaken to define the most effective dose and route of administration (ROA) of an oligotropic AAV (AAV/Olig001; (WO2014/052789; Powell et al. (2016) Gen. Ther. 23:807-814)) capsid variant in promoting widespread CNS oligodendrocyte transduction in a mouse model of the inherited human leukodystrophy, Canavan disease. Three doses of AAV/Olig001, delivered via four distinct ROA were tested in adult, symptomatic Canavan mice (nur7), and vector spread and transduction quantified two weeks post-transduction by generating stereological estimates of reporter green fluorescent protein (GFP) positive cells in four anatomical regions of interest. The tropism of AAV/Olig001 delivered via each ROA was assessed by scoring the incidence of lineage-specific antigens colabeling with GFP in these same regions to validate oligotropism. ROA employed were intraparenchymal (IP), intrathecal (IT), intracerebroventricular (ICV), and intracisterna magna (ICM). Three doses of vector were administered via each route, 1×10¹⁰, 1×10¹¹, and 5×10¹¹ total vector genomes (VG), with the volume of material delivered constant across all treated cohorts and direct pair-wise comparisons of each undertaken to define the optimal combination of dose and ROA for AAV/Olig001 application to Canavan disease. Six-week old aspartoacylase deficient nur7 mice (Traka et al. (2008) J. Neurosci 28:11537-11549) were employed, representing an acutely symptomatic phase of Canavan disease. The results generated by this study formed a foundation for subsequent preclinical efficacy studies to support the clinical application of AAV/Olig001 to currently intractable white matter diseases, such as Canavan disease.

Materials

AAV/Olig001 Vectors

Two lots of AAV/Olig001 vector containing a constitutive expression cassette for a GFP reporter gene were produced (Lot #7660 and Lot #LAV38A). All vector produced contained a GFP reporter gene driven by a hybrid CMV/chicken β-actin promoter (CBh) flanked by self-complimentary AAV ITRs. Vector was produced by transient transfection of HEK293 cells followed by iodixanol gradient centrifugation and ion-exchange chromatography (Gray et al., (2013) Gene Ther. 20:450-9). Concentration of vector was defined as total numbers of viral vector genomes (vg), determined by qPCR quantification of DNAse-resistant AAV inverted terminal repeat (ITR) sequence in the stock preparation.

Animals

All animals used in this study were generated from a colony maintained at the Rowan School of Osteopathic Medicine animal facility under approved institutional protocols. Founder animals originated from a commercial source (Jackson Laboratories). The nur7 mouse is a well-characterized model of Canavan disease that harbors an inactivating point mutation in the gene encoding for the glial hydrolytic enzyme aspartoacylase (aspa), rendering the protein non-functional (Traka et. al. J. Neuroscience (2008) 28(45)11537-11549). Homozygous nur7 mutant animals were generated from the pairing of heterozygous carrier animals and genotyped using an in-house customized SNP assay and real-time PCR.

AAV/Olig001-GFP, diluted to the appropriate concentration in 0.9% saline, was delivered by stereotaxic injection to 6-week old nur7 mutant mice under inhalation anesthesia (4% induction and maintenance titered to effect. Four treatment cohorts distinguished by each providing a different route of administration (ROA) were generated; intrathecal (IT), intraparenchymal (IP), intracerebroventricular (ICV), and intracisterna magna (ICM). Within each ROA cohort, subgroups of animals defined by vector dose administered by each ROA were established (1×10¹⁰, 1×10¹¹, and 5×10¹¹ total vector genomes).

Thus, for each ROA, three subgroups, defined by dose, were generated, with n=5 animals for each dose at each ROA giving a total of 60 nur7 mice for the study. AAV/Olig001-GFP was administered to anesthetized mice, and for all surgeries, regardless of dose or ROA, a total delivered volume of 5 μL was constant. IP ROA required 5× injections of 1 μL of vector at 5 stereotaxic coordinates, two in each hemisphere to anterior and posterior subcortical white matter (i.e., 4 injections total to the cingulum) and 1 additional injection in cerebellar white matter (to give a total of 5) at a rate of 0.1 μL/min using a digital pump. IT ROA animals received a single 5 μL infusion of vector into the subarachnoid space accessed via lumbar puncture between L5 and L6. ICV ROA animals received two 2.5 μL injections of vector, one in each lateral ventricle at a rate of 0.1 μL/min. ICM ROA animals received 5 μL of vector delivered directly to the CSF via the cisterna magna at a rate of 0.1 μL/min. All animals received 0.5 mL 20% mannitol (ip) 20 minutes prior to surgery. All animals were group-housed for two weeks following AAV/Olig001-GFP delivery then sacrificed for post mortem analyses.

Groups of naïve 2-week and 8-week old wild type and nur7 mice were given systemic BrdU (50 mg/kg, ip) twice a day for two consecutive days then sacrificed on the third day. BrdU was administered at a concentration of 50 mg/kg to animals. Brain tissue sections were processed for BrdU staining, after DNA hydrolysis in 1 M HCL, using a commercially-available antibody (Millipore-Sigma).

Methods

Quantification of Vector Biodistribution by Unbiased Stereology

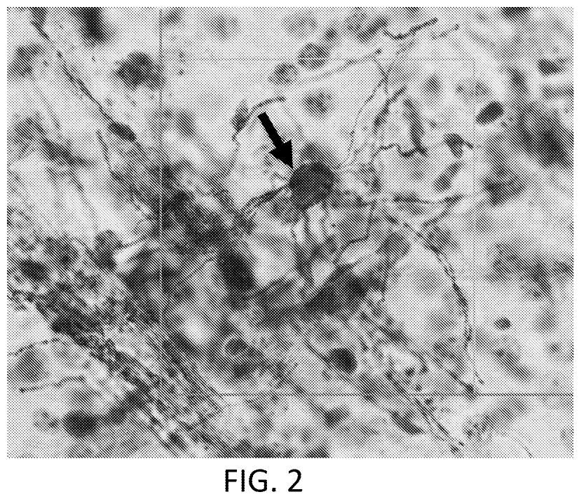

Two weeks after vector surgeries, animals were deeply anesthetized, and brains prepared by transcardial perfusion with 0.9% saline followed by freshly prepared buffered 4% paraformaldehyde. Perfused brains were excised and post-fixed in 4% PFA overnight at 4° C. Fixed brains were cryopreserved and flash frozen in a dry ice/isopentane bath and stored at −80° C. prior to immunohistochemical processing. Serial 40 μm sagittal sections were generated for each brain (144 total sections) and every 4^{th} section stained for GFP using a commercially available antibody (Sigma/Millipore). GFP-positive soma in the cortex, subcortical white matter, striatum, and cerebellum were scored by unbiased stereology using the optical fractionator method (FIG. 2) (West et al. Anat. Rec. (1991) 231:482-97). Stereology software (Stereologer, Stereology Resource Center) coupled to an upright bright field microscope fitted with a motorized stage was used to generate counts of GFP-positive soma within four different regions of interest, namely, the cerebral cortex, subcortical white matter of the corpus callosum and external capsule, striatum, and cerebellum. GFP-positive cells in the sampling faction were reconverted to absolute estimates throughout each region of interest using the formula, ΣQ*(t/h)*(1/asf)*(1/ssf), where ΣQ=particles counted, t=section thickness, h=counting frame height, asf=area sampling fraction, and ssf=section sampling fraction. For all data sets thus generated, intra sample variation was monitored by calculation of the coefficient of error (CE) which satisfied a less than 15% contribution to total variance (CV) threshold to reduce technical noise masking true biological variance between samples. Significant differences in mean group mean estimates of N were determined by unpaired two-tailed student's t-test with a threshold significance of p≤0.05.

Quantification of Vector Tropism

Vector tropism in AAV/Olig001-GFP transduced brains was quantified by scoring for lineage-specific antigen colabeling with GFP fluorescence. Alternate sections were processed for NeuN (which is present in most CNS and PNS neuronal cell types of vertebrates), GFAP (glial fibrillary acidic protein), or Olig2 (oligodentrocyte lineage transcription factor 2) immunohistochemistry using commercially available antibodies (Sigma/Millipore) to label neurons, astrocytes, and oligodendrocytes, respectively. Scanning confocal microscopy was employed to generate multipoint image stacks throughout each region of interest. NIS-Elements Advanced Research software (Nikon) was used to count total GFP-positive cells and the number of GFP-positive cells colabeling with each lineage-specific antigen (Olig2 and NeuN) in each stack. Numbers were collated for each individual brain (8 serial sections in total sampled from each brain with a sample interval of 4). ROI in individual sections were outlined by software and individual points placed every 200 um2 sampled at high magnification to score for both GFP immunofluorescent soma and GFP/Olig2 or NeuN positive cell bodies. The total number of GFP-positive soma co-labelling with either Olig2 or NeuN was calculated by dividing the number of GFP-positive soma by lineage-specific co-labeling in each series of sections. Means for each ROA were calculated (n=5 animals).

Results

Intraparenchymal (IP) ROA Dose Response

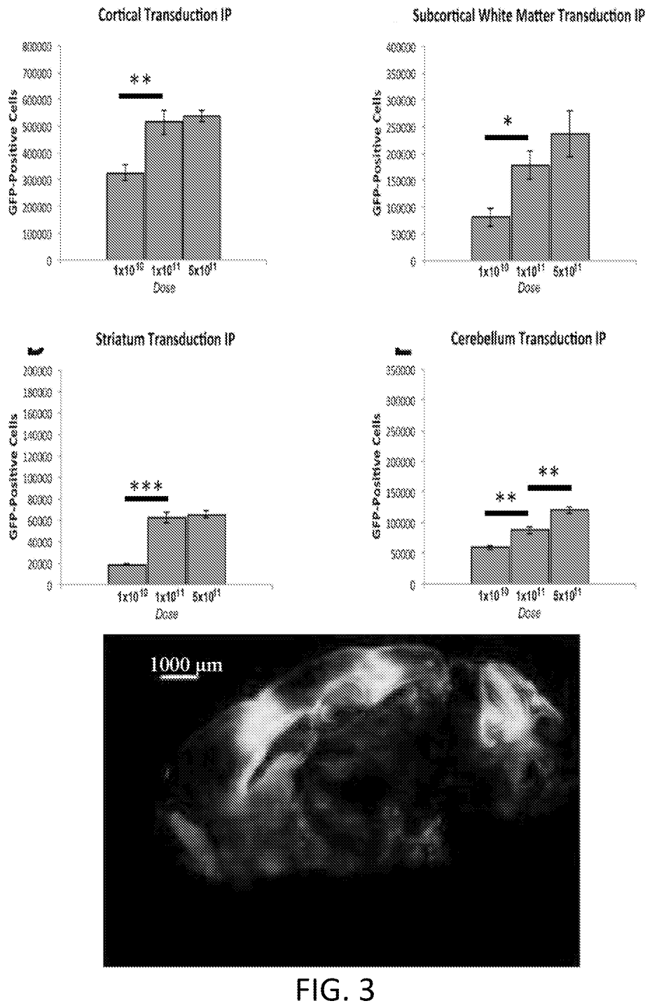

IP ROA animals were given 5 individual injections targeting subcortical white matter in both hemispheres and the cerebellum. Treated animals were sacrificed 2-weeks post-vector administration (8 weeks of age) and brains were processed for GFP immunohistochemistry and GFP-positive soma in the cortex, subcortical white matter, striatum and cerebellum were scored using unbiased stereology using the optical fractionator to provide absolute estimates of transduced cells in each region of interest. All three doses of AAV/Olig001-GFP administered resulted in significant levels of transduction of cells throughout the brain. (FIG. 3) Within the cortex, an increase in transduced cell numbers being significant between the 1×10¹⁰ and 1×10¹¹ vg doses (+1.6-fold, p=0.0096), but no further increase at the highest 5×10¹¹ dose was evident (p=0.659), suggesting saturation (FIG. 3). In subcortical white matter of the corpus callosum and external capsule, high levels of transduction were evident, with positive cells most concentrated immediately adjacent to the four injection sites. Subcortical white matter GFP-positive cells were also increased in a dose-dependent manner, with the 2.2-fold increase from the 1×10¹⁰ to 1×10¹¹ dose being statistically significant (p=0.0144), but the 1.3-fold increase from 1×10¹¹ to 5×10¹¹ failed to reach statistical significance (p=0.283). Modest striatal transduction was evident. The 3.4-fold increase from 1×10¹⁰ to 1×10¹¹ was highly significant (p=4.38×10⁻⁵), but no further increase in transduction was evident at the 5×10¹¹ dose (p=0.706). Transgene expression within the cerebellum was limited to an area immediately surrounding the single injection site with both the 1×10¹¹ and 5×10¹¹ doses resulting in significant increases over the preceding dose (1×10¹¹: 1.5-fold increase [p=0.0016]; 5×10¹¹: 1.4-fold increase [p=0.0019]). The cortex presented the highest numbers of transduced cells (513,477), followed by subcortical white matter (178,362), cerebellum (86,820), and finally striatum (62,706). GFAP co-labeling was less than 2%.

Intrathecal (IT) ROA Dose Response

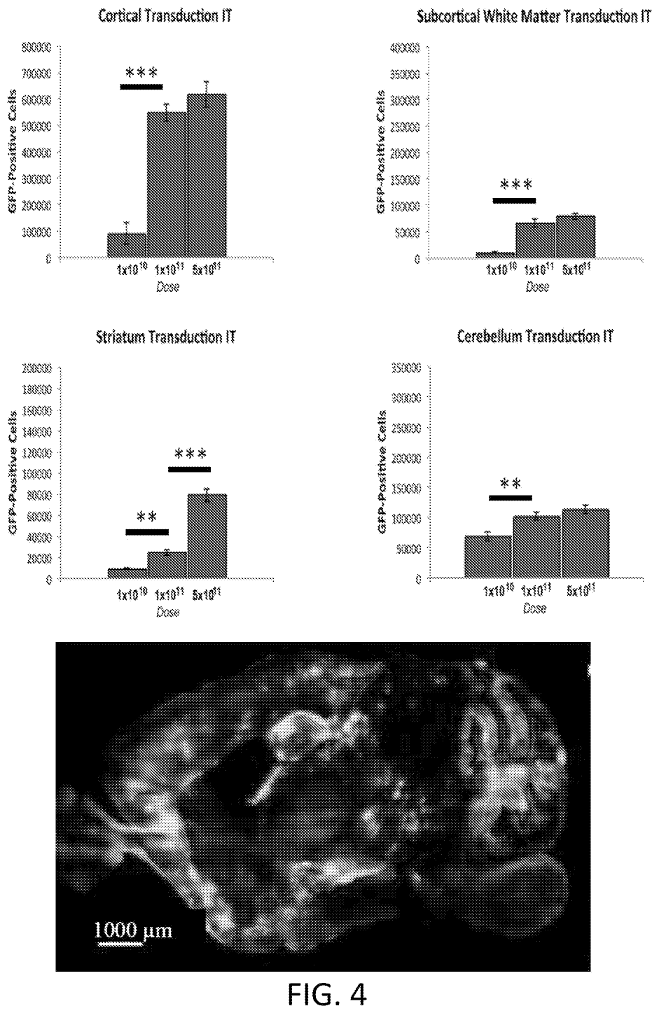

IT administration of AAV/Olig001-GFP resulted in excellent distribution of transgene expression throughout the brain with the exception of subcortical white matter of the corpus callosum and external capsule (FIG. 4). A highly significant increase in cortical transduction from 1×10¹⁰ to 1×10¹¹ vg was evident (6.1-fold increase, p=0.000026), with no significant increase at the 5×10¹¹ vg dose (p=0.273). While the distribution of GFP expression in the IT ROA cortex was excellent, the intensity of expression was somewhat reduced compared to IP brains. Appreciable GFP expression was noted, unsurprisingly, in the lumbar region of the spinal cord, suggesting some dilution of the vector by spinal cord tissue en route to the brain. The most striking observation in IT ROA brains was the paucity of transgene expression in the corpus callosum and external capsule. While a very significant increase in GFP-expressing white matter tract cells were seen when the dose was increased from 1×10¹⁰ to 1×10¹¹ (6.3-fold increase, p=0.00021), absolute number of transduced white matter cells in IT ROA brains was relatively modest. Mean numbers of positive cells in this white matter tract region of the brain were 64,970 at the 1×10¹¹ dose, compared with 178,362 for IP brains at the same dose. As with the cortical ROA, further increasing the dose from 1×10¹¹ to 5×10¹¹ in IT ROA brains did not significantly increase numbers of transduced white matter tract cells (p=0.203).

The striatum presented with a dose responsive increase in transduced cells at each successive dose. Increasing the dose from 1×10¹⁰ to 1×10¹¹ resulted in 2.7-fold more GFP-positive cells in the striatum (p=0.001). A subsequent increase to the 5×10¹¹ dose saw a 3.2-fold increase in positive cells (p=0.000037), which resulted in numbers comparable to IP ROA brain striatal transduction (IT at 5×10¹¹ mean 79,444, IP at 5×10¹¹ mean 65,203).

The IT ROA resulted in strong cerebellar transgene expression, with a significant 1.5-fold increase observed when moving from the 1×10¹⁰ to the 1×10¹¹ dose (p=0.0064), but no further increase was seen at the 5×10¹¹ dose. Cerebellar transduction was comparable to IP ROA brains, and was slightly, but not significantly higher at the 1×10¹¹ and 5×10¹¹ doses.

Intracerebroventricular (ICV) ROA Dose Response

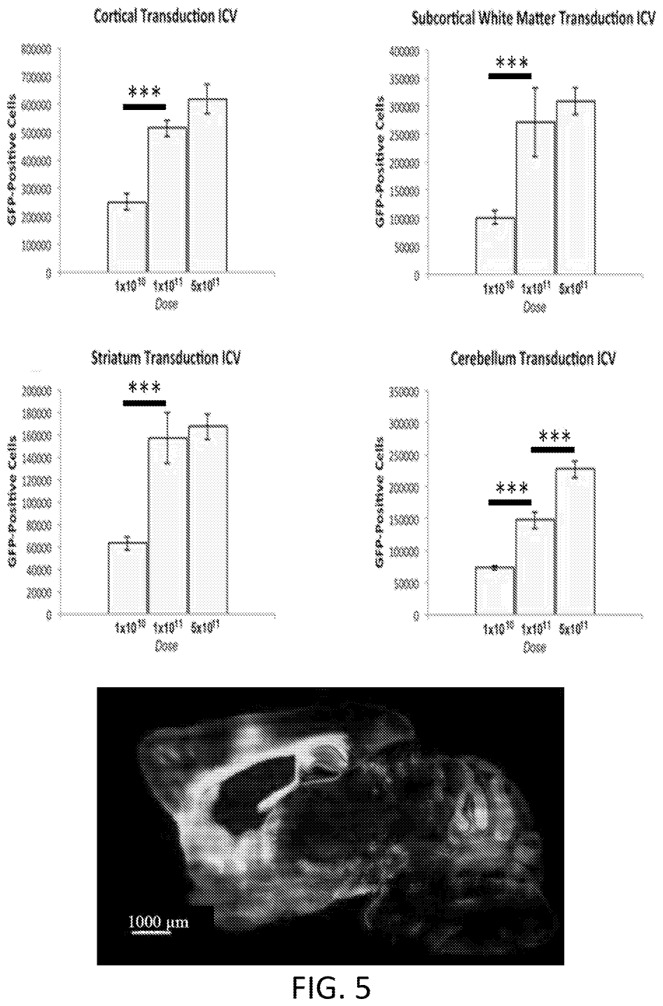

ICV administered AAV/Olig001-GFP resulted in prominent transgene expression throughout all areas of interest, with particularly robust transduction of subcortical white matter notable (FIG. 5). All regions of interest displayed a dose responsive increase in numbers of transduced cells when the dose was increased from 1×10¹⁰ to 1×10¹¹, although there were subtle non-significant increases in most regions at the 5×10¹¹ dose compared to the 1×10¹¹ dose, except for the cerebellum. Cortical transgene expression was comparable to IP ROA brains, with a 2-fold increase in transgene positive cells when dose was increased from 1×10¹⁰ to 1×10¹¹ (p=0.00029), and a further 1.2-fold increase following administration of the 5×10¹¹ dose failing to reach statistical significance (p=0.123).

Subcortical white matter transduction in ICV brains was substantial, with an observed 2-fold increase in GFP-positive white matter tract cells when dose was increased from 1×10¹⁰ to 1×10¹¹ (p=0.00052). A modest non-significant increase was observed at the highest 5×10¹¹ dose (p=0.334). Subcortical white matter transduction in ICV brains at the 1×10¹¹ dose was significantly increased 1.5-fold relative to IP brains (p=0.041), and increased 4.2-fold relative to IT ROA brains (p=0.0001).

A very similar pattern of transgene expression was seen in the striatum of ICV dose cohorts, with a significant 2-fold increase in GFP-positive cells observed when dose was increased from 1×10¹⁰ to 1×10¹¹ (p=0.000043), but no significant further increase at the 5×10¹¹ dose (p=0.537). Robust striatal transgene expression was evident in ICV brains, with an increase in GFP-positive cells in this region of 2.5-fold over IP brains (p=0.00004).

Cerebellar ICV transduction was robust, with dose dependent increases in GFP-positive cells observed at both successive higher doses (+2-fold at 1×10¹¹, p=9.56×10⁻⁶; +1.5-fold at 5×10¹¹, p=0.00073). There was a 1.7-fold increase in GFP positive cells relative to IP brains (p=0.0001), and a 1.4-fold increase over IT numbers (p=0.0013) at the 1×10¹¹ dose. The cerebellum was the only region that presented with a further appreciable increase in GFP-positive cells in ICV ROA brains.

A prominent point of difference between IP and ICV ROA brains that was evident throughout the sampling process was the greater distribution of vector in the ICV groups. Transgene expression at injection sites was more intense in IP brains but diluted rapidly from the site. By contrast, ICV transgene expression was relatively evenly distributed over a far greater area of the brain.

Intracisterna Magna (ICM) ROA Dose Response

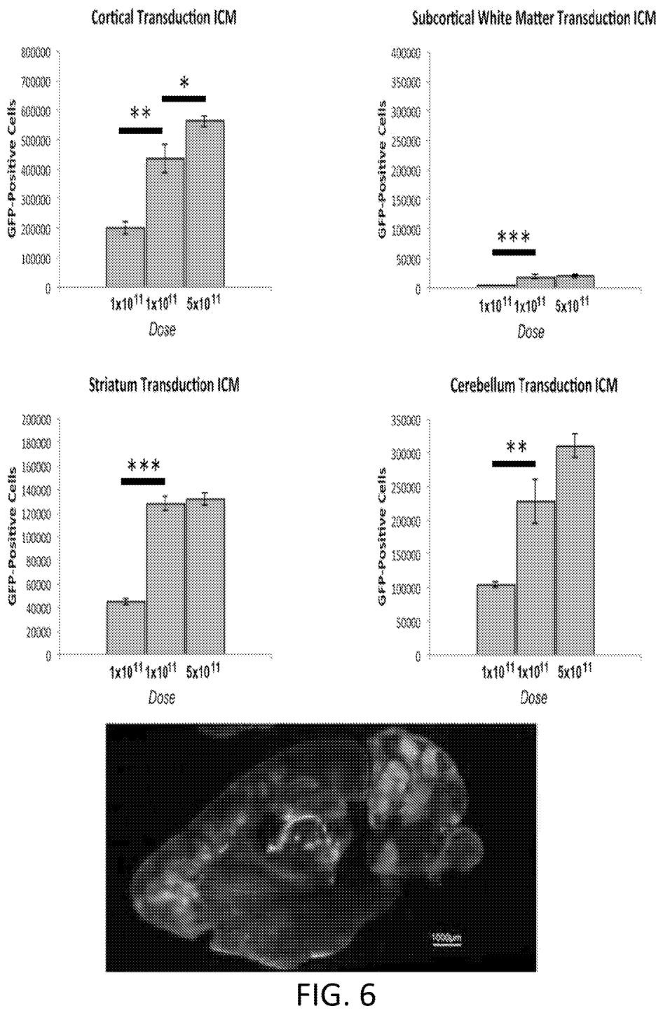

ICM administration of AAV/Olig001-GFP resulted in relatively widespread transgene yet modest transgene expression in the cortex, striatum and cerebellum. However, like IT ROA brains, there was an absence of significant transgene expression in subcortical white matter of ICM brains (FIG. 6). Cortical transgene expression was dose-responsive, with each successively higher dose resulting in a significant increase in GFP-positive cells (1×10¹¹, 2.2-fold increase p=0.018; 5×10¹¹, 1.3-fold increase p=0.043). Both the striatum and cerebellum saw significant increases in GFP-positive cells at the 1×10¹¹ dose (p=2.49×10⁻⁶, and p=0.0062 for striatum and cerebellum, respectively), with the highest 5×10¹¹ dose resulting in further increases in positive cells in the cerebellum only (p=0.061). Transduction of subcortical white matter tracts by the ICM ROA was decidedly modest. While increasing administered dose from 1×10¹⁰ to 1×10¹¹ resulted in a significant increase in GFP-positive cells (p=0.00086), the actual number of transgene positive cells present was relatively negligible.

Relative to ICV brains, ICM subcortical white matter GFP-positive cells were reduced 14.2-fold (ICV mean 271,274; ICM mean 18,996, p=0.00002) and relative to the next lowest subcortical white matter transduced ROA group of animals, IT, was reduced 3.4-fold (IT mean 64,970), making ICM the least effective ROA in transducing white matter. Distribution across other regions of interest were comparable to other ROA treatment groups, with no significant difference in cortical transduction evident when compared to all three other ROA. Striatal transduction via ICM was slightly reduced when compared to ICV ROA (p=0.043). Striatal ICM GFP expression was significantly greater than both IP (+2.0-fold, p=0.00005) and IT (+5.1-fold, p=0.0000005) at the 1×10¹¹ dose. ICM brains presented with the highest numbers of transduced cerebellar cells of any of the four ROA examined. At the 1×10¹¹ dose, cerebellar ICM transduction was increased over ICV by 1.5-fold (ICM mean 228,282; ICV mean 157,203), by 2.6-fold over IP, and by 2.2-fold over IT.

Routes of Administration (ROA) Compared

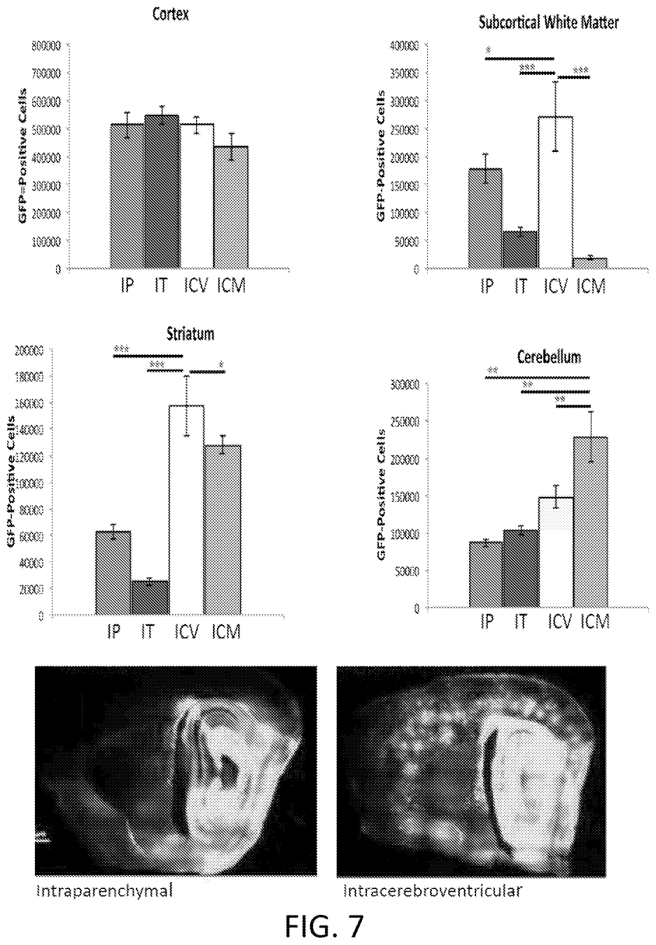

For all ROA explored here, in all regions of interest, increasing vector dose from 1×10¹⁰ to 1×10¹¹ elicited a 2-3 fold increase in transduced cell numbers, while a further dose escalation to 5×10¹¹ resulted in negligible increases in transduced cells overall. A direct comparison of all four ROA at the 1×10¹¹ dose in each region of interest revealed clear differences in absolute numbers of transduced cells in all four ROI (FIG. 7). Numbers of transduced cells in the cortex of brains transduced with 1×10¹¹ vector genomes did not differ significantly with each ROA, with all resulting in an average of 44,000-50,000 positive cell soma. In contrast there was a clear advantage to the ICV ROA in subcortical white matter, where transduced cell numbers were significantly higher in ICV brains than any other group. ICV and IP-transduced brains gave the highest and second highest numbers of transduced white matter tract cells respectively. The average 2.7×10⁵ positive cells in white matter tracts of brains transduced with 1×10¹¹ AAV/Olig001-GFP vector genomes via the ICV ROA was significantly greater than the average 1.8×10⁵ positive cells present in IP brains subject to the same dose (p=0.041).

IT and ICM ROA were both inefficient at transducing subcortical white matter cells, with the average 1.9×10⁴ cells in the ICM group a significant 14-fold less, and the IT group 4-fold less (p=0.0001) than the average 2.7×10⁵ positive cells in the ICV group (p=0.000083). This may be of concern in a disease model system that presents with deficits in myelin.

The ICV route also results in efficient transduction of cells in the striatum with higher numbers of GFP-positive cells in ICV brains than all other ROA (ICV vs. IP p=3.68×10⁻⁵; ICV vs. IT p=1.61×10⁻⁵; ICV vs. ICM p=0.043). The efficiency of transduction of the cerebellum was comparable across all of IP, IT, and ICV ROA, but ICM brains presented with the highest numbers of transduced cerebellar cells (ICM vs. ICV p=0.045).

Although IP and ICV ROA brains were comparable in absolute numbers of cells transduced by AAV/Olig001-GFP in specific regions, the bulk of positive cell counts in IP brains were the product of sections immediately adjacent to injection sites, while positive cells in ICV brains were relatively evenly distributed. Systemic non-random stereological sampling allows for the identification of variance between sections sampled from individual brains (intrasample variance), and is represented as the coefficient of error (CE) in a dataset, calculated by the standard error of the mean of repeated estimates divided by the mean. CE is one half of total variance in a sampled population, with true biological variance (CV), or difference in the mean between individual brains, constituting the other half. The mean CE for individual IP brains was calculated as ˜12% of total variance, while that for ICV brains was ˜3%, meaning GFP-positive cells were more evenly distributed across all sections sampled in ICV brains. In IP brains, actual numbers of positive cells in individual sections sampled became fewer the further laterally from injection sites the sampled section was, while positive cells numbers in ICV brains were consistently closer to the intrasample mean in all sections sampled. The net result of this difference was a greater spread of vector in ICV ROA brains relative to IP brains, particularly in the cortex and subcortical white matter (FIG. 7).

Conclusion

Using four distinct ROA, a combination of dose and ROA conducive to global CNS oligodendrocyte transduction in acutely symptomatic animals that closely model the Canavan brain at time of diagnosis was defined. Administration of AAV/Olig001-GFP vector resulted in greater than 70% oligotropism in all regions of interest, bar the cerebellum, without the need for lineage-specific expression elements. A dose-dependent increase in transgene-positive oligodendrocytes was apparent in all ROA, with an intracerebroventricular ROA promoting higher numbers of transduced white matter tract cells while maintaining a greater than 90% oligotropism in this key region of interest. These data emphasize the capsid-cell surface interaction as a primary determinant of oligotropism, which is most relevant to clinical application to abnormalities specific to oligodendrocytes, such as Canavan disease. These data also demonstrate that the Olig001 capsid has a potential therapeutic capsid for the treatment of oligo-dendrocyte-related diseases, disorders and/or conditions, including Canavan disease.

### Example 3: Vector Tropism by Route of Administration (ROA)

A distinguishing characteristic of AAV/Olig001 is its clear oligotropism as compared to other AAV capsid variants (Powell et al. (2016) Gen. Ther. 23:807-814; Francis et al. (2016) Neurobiol. Dis. 96:323-334). For application to Canavan disease, a white matter disorder by definition, AAV/Olig001 vectors must be capable of exhibiting this tropism when applied by different ROA. Oligotropism may vary due to variables such as age of intervention (Gholizadeh et al. Hum. Gene Ther. Methods (2013) 24:205-13; Foust et al. Nature Biotech. (2009) 27:59-65), and while previous work has documented the oligotropic potential of AAV/Olig001 in neonatal nur7 mice (Francis et al. Neurobiol. Dis. (2016) 96:323-334), translation of this tropic potential to older, symptomatic animals remains untested. To this end, all four ROA at the 1×10¹¹ dose employed in Example 2 were assessed for potential impact on vector tropism in 6-week old animals. The cortex, subcortical white matter, striatum, and cerebellum used for the generation of absolute numbers of GFP-positive cells were analyzed for co-labeling of GFP transgene with the lineage specific antigens Olig2 (i.e., target specific labeling for oligodendrocytes) and NeuN (i.e., target specific labeling for neurons).

Results

All four ROA generated comparable results, with oligotropism intact. Non-oligodendrocyte transgene expression was attributable to neurons, with very few astrocytes observed expressing GFP in all 4 ROA cohorts (<5%).

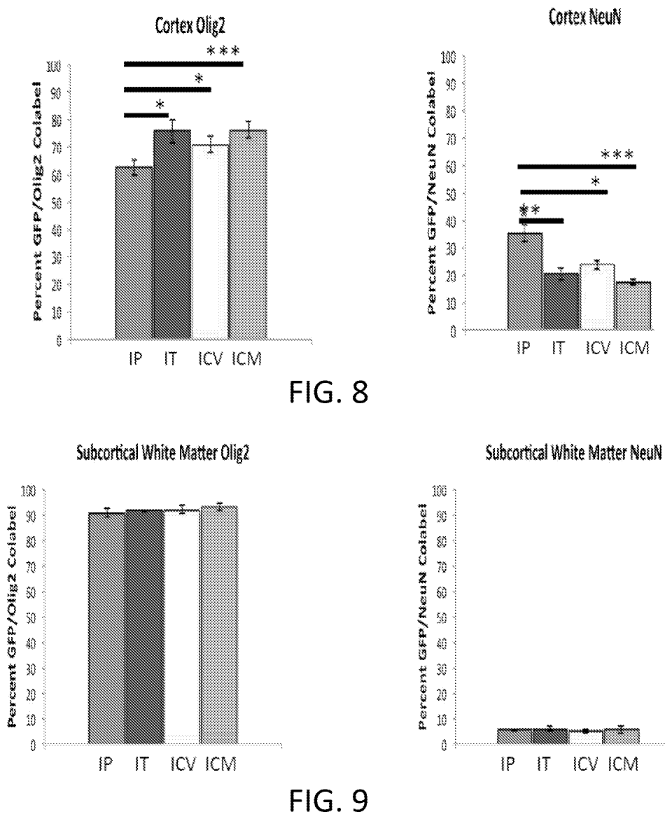

Cortical co-labeling of Olig2 with GFP was comparable amongst IT, ICV and ICM ROA with the percentage of total GFP positive cells co-labeling with Olig2 consistently around 75%. In IP transduced brains, about 62.3% of GFP-positive cells co-labeled with Olig2, a small but significant reduction (FIG. 8). GFP-positive cells within these same brains co-labeling with NeuN essentially accounted for the remaining transduced cortical population (35.1%). All three of IT, ICV and ICM ROA presented around 20% NeuN co-labeling. In IT ROA brains 75.5% of cortical GFP-positive cells co-labelled with Olig2 and 20.2% with NeuN. ICV ROA brains presented with 70.8% oligotropism and 23.6% neurotropism in the cortex, while the cortex of ICM brains manifest 76% GFP co-labelling with Olig2, and 17.4% with NeuN. The difference in oligotropism manifest amongst the 4 different ROA was small, but the IP ROA did present with a significant increase in NeuN co-labelling (p=0.0043 vs. IT; p=0.0119 vs. ICV; p=0.00059 vs. ICM) that coincided with slight but significant reductions in Olig2-co-labelling relative to the other 3 ROA (p=0.026 vs. IT; p=0.048 vs. ICV; p=0.0085 vs. ICM), suggesting that IP ROA promoted small increases in neurotropism at the expense of oligotropism. Again, the IP ROA was notable for an increase in NeuN co-labeling (+1.5-fold, p=0.012), suggesting reduced Olig2 co-labeling is accounted for by increased neuronal transduction in this ROA. Most of the GFP-NeuN co-labeling in IP ROA brains was clustered around injection sites, indicating saturating quantities of AAV/Olig001-GFP immediately adjacent to the site of injection.

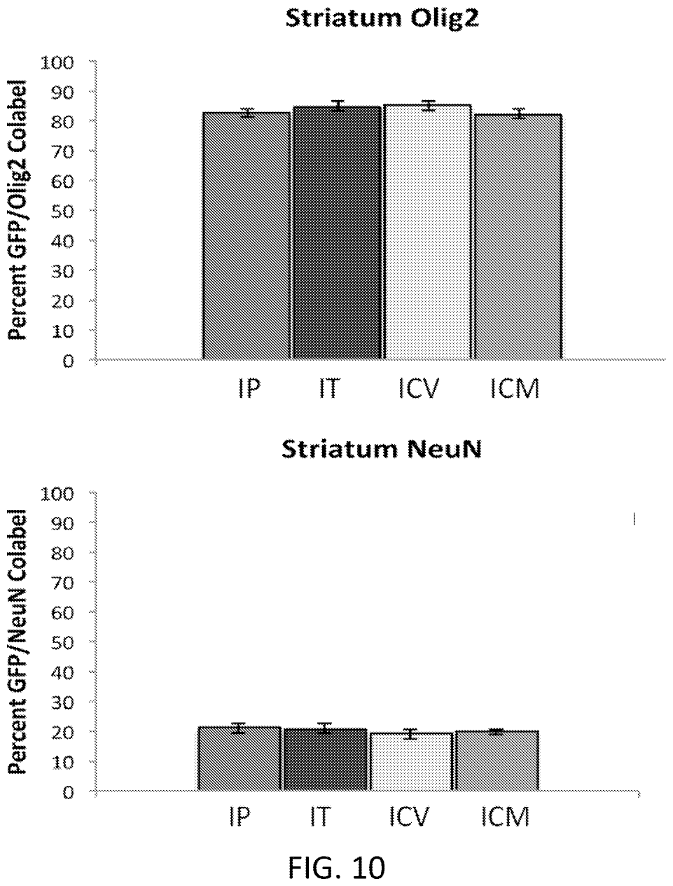

Subcortical white matter co-labeling of Olig2 with GFP was >90% in all four ROA (FIG. 9). Co-labeling of NeuN with GFP was <6% in all four ROA. No significant difference in percentage co-labeling with either antigen was observed between ROA, indicating a strong preference for oligodendrocytes in white matter-rich regions regardless of ROA. By ICV transduction, there was near ubiquitous Olig2 co-labeling and absence of NeuN co-labeling in the corpus callosum.

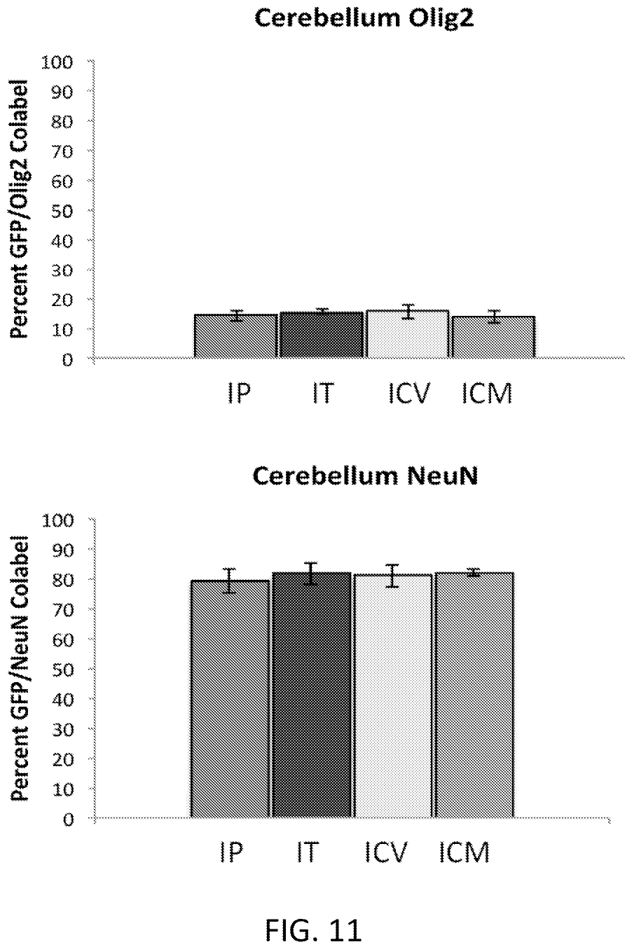

Striatum co-labeling of Olig2 with GFP was comparable amongst all ROA with the percentage of total GFP positive cells co-labeling with Olig2 >80% (FIG. 10). The remaining GFP positive cells in the striatum (<20%) co-labeled with NeuN.

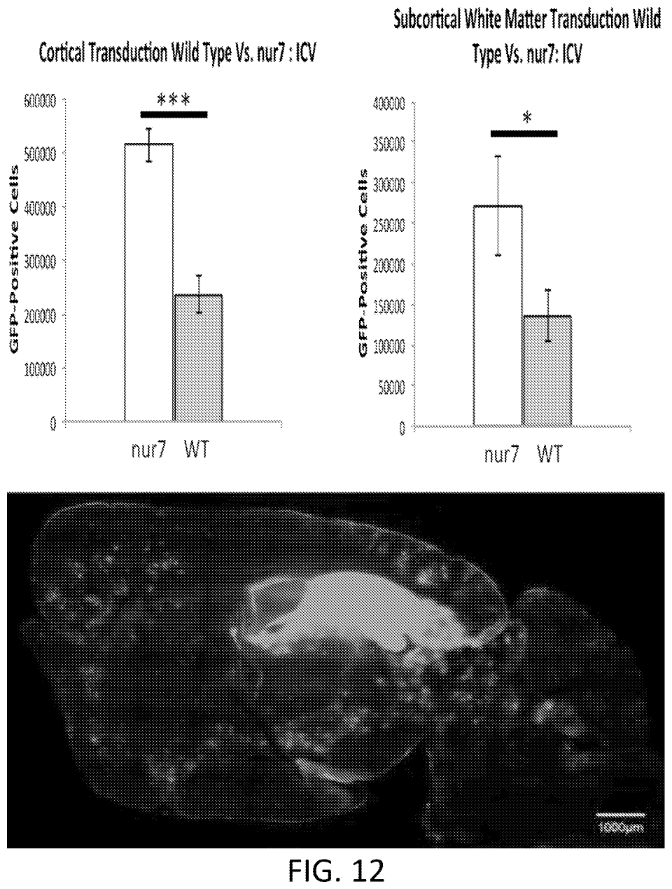

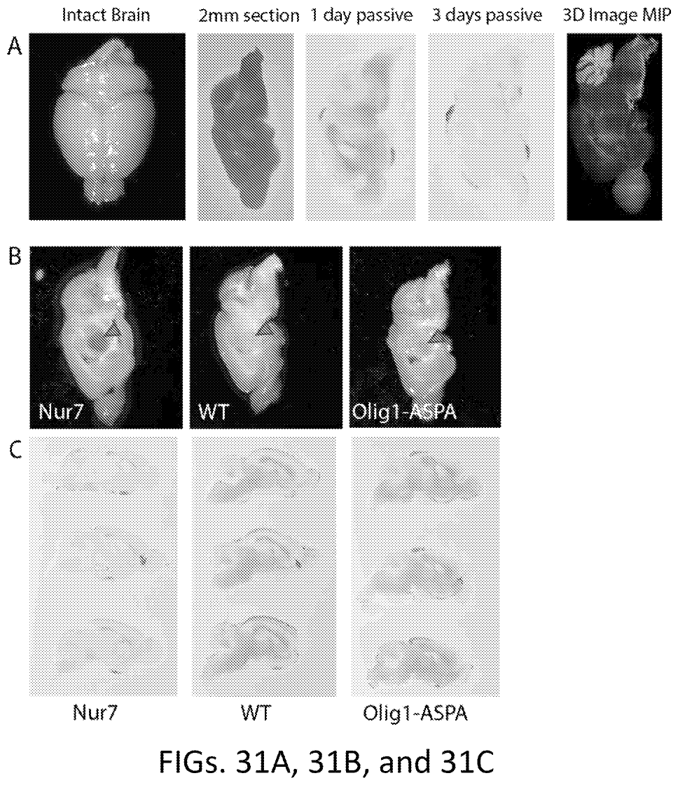

Cerebellar co-labeling demonstrated opposite ratios of Olig2 and NeuN in all four ROA as compared with the other regions of the brain that were studied. The percentage of Olig2 co-labeling was 10% of total GFP positive cells in all four ROA (FIG. 11). Transgene expression was dominated by neurons in the cerebellum, which accounted for over 80% of GFP expressing cells. No significant difference in percent co-label with either antigen was observed between ROA cohorts in the cerebellum. Large purkinje neurons in the granule cell layer were intensively GFP positive (FIG. 29C) with only sporadic Olig2/GFP co-labeling within the cerebellar white matter tracts. This was in contrast to the near 100% oligotropism observed in subcortical white matter (FIG. 29B), and the 70% to 80% oligotropism observed in comparatively neuron-dense regions such as the cortex and striatum (FIG. 29D). Total GFP-positive cells scored for each ROA at the 1×10¹¹ dose were ranked in order of highest to lowest mean of total GFP positive cells (+/−sd) with n=5: ICV 1104256.4 (106816.96); IP 841365.6 (121722.7); ICM 815486.9 (106979.7); IT 742143.1 (79496.5).

The ICV ROA results in the highest number of total GFP-positive cells (sum of all ROA counts in individual brains), which were 1.3-fold more than the next ranked ROA, IP (p=0.0067). Total numbers in ICV brains were significantly increased over all ROA, including ICM (p=0.0027) and IT (p=0.0003). Numbers of cells in IP ROA brains were not significantly increased over either ICM (p=0.730) or IT numbers (p=0.165), marking the ICV ROA clearly superior in total cells transduced. Approximately 75% of the difference in overall GFP-positive cell numbers between ICV and IP cohorts (˜262,891) was accounted for by subcortical white matter (35%) and striatal (36%) ROIs, which manifested >80% oligotropism in both ROA cohorts. This means that ICV brains contained somewhere in the region of at least 210,000 more transduced oligodendrocytes than IP brains. If this analysis is restricted to within subcortical white matter, an ROI presenting >90% oligotropism by all ROA, then at least 83,000 more transduced oligodendrocytes per brain are to be expected when administering AAV/Olig001 via the ICV ROA. When assessed against the ROA cohort presenting the poorest levels of GFP transgene expression, the ICM cohort, ICV administration resulted in an increase in AAV/Olig001-transduced oligodendrocytes of over 200,000 cells per brain.

The adult mammalian CNS is known to harbor significant numbers of oligodendrocyte precursor cells in white matter (Dawson et al. Mol. Cell Neurosci. (2003) 24:476-488), and evidence of attempted remyelination in juvenile nur7 in the form of an increased turnover of immature oligodendrocytes (Francis et al. J. Cerebral Blood Flow Metabolism (2012) 32:1725-36) has previously been shown. Given that white matter has a significant capacity for remyelination, even in the adult brain, the persistence of a resident population of immature oligodendrocytes in adult nur7 white matter must be considered an ideal target for an oligotropic gene delivery vector.

In order to assess relative numbers of proliferating oligodendrocyte progenitors/immature oligodendrocytes, both nur7 and wild type mice were given systemic BrdU twice a day for two days and sacrificed, on the third day to processes for BrdU/Olig2 co-labeling (FIG. 29E-G). BrdU administration was initiated in both 2 and 8 week old cohorts to quantify the possible persistence of proliferating oligodendrocytes in young and adult brains. Counts of BrdU-positive cells in the corpus callosum and external capsule of genotype cohorts at each age revealed a significant 1.8-fold increase in BrdU-positive cells in 2-week old nur7 brains relative to wild type (p=0.029) and a 1.6-fold increase in nur7 brains at 8 weeks (p=0.034) (FIG. 29F). The vast majority of BrdU cells in nur7 white matter, at both ages, co-labeled with Olig2, indicating the persistence of proliferating progenitor/immature oligodendrocytes in white matter of adult symptomatic nur7 mice. A subset of three 6-week old nur7 mice were given systemic BrdU for two days prior to transduction with 1×10¹¹ vg of AAV/Olig001-GFP, and these animals were sacrificed 2 weeks-post transduction for evidence of transduction of proliferating cells in white matter tracts. Numerous BrdU/GFP co-labelled cells were observed in white matter tracts of these animals, indicating the successful transduction of resident progenitor/immature cells.

A group of healthy wild type animals, age matched to nur7 ROA cohorts (i.e. 6 weeks of age) were transduced with 1×10¹¹ vg of AAV/Olig001-GFP via the ICV ROA, and sacrificed 2 weeks later for generation of stereological estimates of GFP-positive cells within the cortex and subcortical white matter tracts (FIG. 8). Estimates of GFP-positive cells revealed a significant 2-fold reduction in both the cortex and subcortical white matter of wild type brains as compared with nur7 brains (p=0.00032 and p=0.0116 for each respective ROI). Subcortical white matter GFP transgene expression in wild type brains was very much restricted to regions immediately surrounding the lateral ventricles in wild type brains, while cortical expression, although reasonably diffuse, was very modest in absolute number of transduced cells.

Conclusions

Examples 2 and 3 demonstrate that intracerebroventricular (ICV) route of administration of the AAV/Olig001 GFP vector provided the best combination of vector spread and oligodendrocyte tropism. Crucially, this ROA appears well suited to the transduction of subcortical white matter, the tissue impacted by Canavan disease pathology. Thus, the ability to transduce hundreds of thousands of cells, and maintain a near 100% tropism for oligodendrocytes, confers a significant advantage to AAV/Olig001 over other AAV capsids. Four to six week old nur7 corpus callosum/external capsule have approximately 1,500,000 Olig2-positive cells, thus, administration of a 1×10¹¹ dose of AAV/Olig001 vector via the ICV ROA has the potential to transduce ˜20% of the resident oligodendrocyte population. It should be noted that white matter tracts of nur7 mice present with evidence of attempted remyelination and contain significant numbers of proliferating oligodendrocyte progenitors. Given that a single oligodendrocyte is capable of myelinating multiple axons, the potential for remyelination following transduction of white matter with a therapeutic AAV/Olig001 vector is significant.

Other CSF-targeted ROA, namely IT and ICM, presented with relatively poor white matter tract transduction, and would not be a first choice for consideration as a therapeutic ROA. IP brains approached comparable levels of transduction in terms of numbers of cells transduced, but the majority of these cells were concentrated about injection sites. Cells at these sites likely had a greater vector genome copy number/cell of any other ROA, but vector spread away from these sites was markedly lower as compared to the ICV ROA. The broader distribution of the GFP transduction by ICV administration is advantageous in the appropriate balance may be achieved between the number of cells transduced and the number of copies of the vector per transduced cell.

Indeed, the intense concentration of transgene expression in IP brains in Examples 2 and 3 was associated with a small but significant reduction in oligodendrocyte tropism and a balancing increase in neurotropism within the cortex. This indicates that saturating a region with AAV/Olig001 may result in a decrease in oligodendrocyte specificity. It should be noted that cortical oligodendrocytes in nur7 mice of the of animals used in the present study are reduced in number from wild type and present with evidence of stress and apoptosis (Francis et al. (2012) J. Cereb. Blood Fl. Metab. 32:1725-1736), which may be expected to impact transduction efficiency.

Vector tropism in all regions of interest was 75-90% oligotropic, with the exception of the cerebellum. This region presented with >80% neurotropism in all ROA groups. Particularly strong transgene expression was seen in granule layer purkinje neurons. The reason for this apparent reversal of tropism is not readily apparent, but the cerebellum is clearly a distinct anatomical entity with respect to resident cell types. Purkinje cells within the cerebellum express Olig2 at low but appreciable levels, and it is possible that the AAV/Olig001 capsid has a markedly different interaction with the Purkinje neurons surface than the surface of other neurons in other regions of the brain.

The current Examples show that AAV/Olig001 promotes robust oligodendroglial transgene expression throughout the brain of nur7 Canavan disease mice, with the important exception of the cerebellum. In all other areas of the brain, >70% oligotropism was achieved without the need for a lineage specific promoter. The inherent affinity of the AAV/Olig001 capsid for the oligodendroglial surface is a significant advantage over selective promoter use in other non-oligotropic capsid serotypes as it ensures that as close as possible to the total dose of vector delivered will express in target cells. These data identify advantages of distinct ROA for targeting white matter in the brain, with the ICV ROA demonstrating applicability to pre-clinical efficacy studies in symptomatic adult nur7 mice as a model for the treatment of Canavan disease.

### Example 4: Difference in Efficiency of AAV/Olig001-GFP Transduction Between Wild Type and nur7 Brains

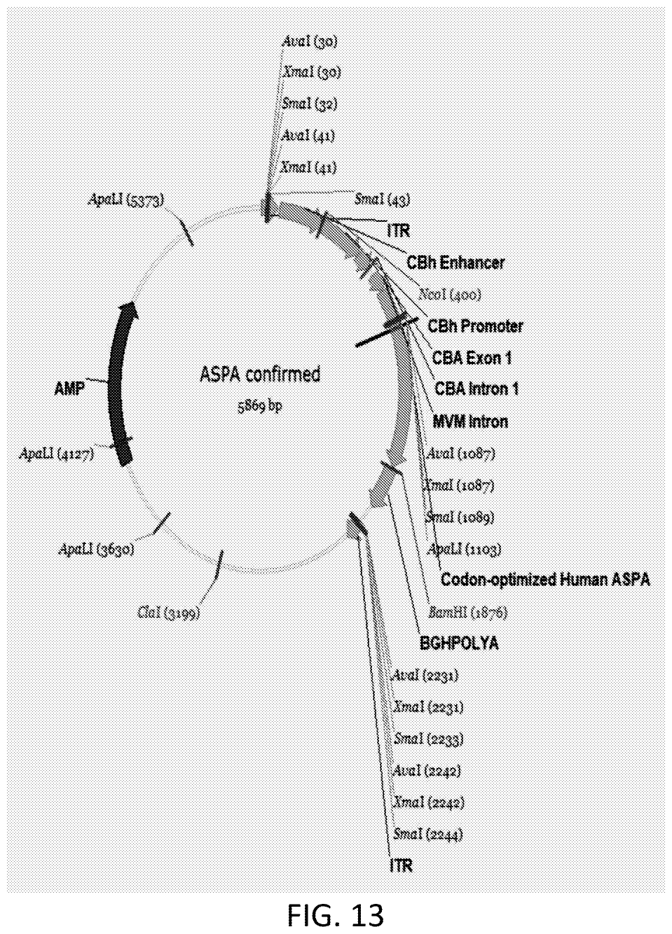

The nur7 mouse model of Canavan disease manifests symptoms of gross motor dysfunction at 2 weeks of age. By 6 weeks of age, the nur7 brain has suffered significant cell loss, loss of white matter and is extensively vacuolated. The 6-week nur7 brain is therefore a markedly different microenvironment than a healthy brain, possibly influencing AAV/Olig001-GFP spread and transduction. Indeed, in a cohort of 6-week old wild type mice, administration of the 1×10¹¹ dose via the ICV ROA resulted in a significantly reduced level of transduction in the cortex and subcortical white matter (FIG. 12) (n=5 animal for each group, mean+/−sem is shown, *p≤0.05, **p≤0.01) as compared to the level of transduction in the brains of nur7 mice.

Stereological estimates of GFP-positive cells in the cortex and subcortical white matter demonstrated a significantly reduced incidence of transgene expression (at least 50% reduction) in the wild type brain. Intense GFP fluorescence is restricted to areas immediately adjacent to lateral ventricles, with modest cortical and subcortical white matter GFP fluorescence signal in the wild type brain. Transgene expression in the cerebellum was poor. These data indicate genotype-specific effects on AAV/Olig001 spread and transduction efficiency. Also, because the nur7 brain, like the human Canavan brain, is heavily vacuolated, has excessively large ventricles, and has profoundly elevated NAA, these signs and symptoms may potentially influence vector spread and biodistribution of a human AAV/Olig001 therapeutic.

### Example 5: In Vivo Administration of AAV/Olig001-ASPA to Nur7 Mice Improves Rotarod Performance

Methods

6-week old nur7 mice were administered a dose of AAV/Olig001-ASPA comprising the codon-optimized ASPA sequence of SEQ ID NO:2. The expression plasmid encoding the codon-optimized ASPA and regulatory elements is shown in FIG. 13. A total dose of 2.5×10¹¹, 7.5×10¹⁰ or 2.5×10¹⁰ vg was administered via the intracerebroventricular (ICV) route of administration (ROA). Vector for all dose cohorts was delivered in a total volume of 5 μl, with 2.5 μl injected in the lateral ventricle of each hemisphere of the brain. A control cohort of age-matched nur7 animals was generated by injection of an equivalent volume of physiological saline via the same ROA. Age-matched naïve wild type animals were used as a calibration reference for all motor function testing. Two weeks after administration of vector, animals were tested once a month for four months for latency to fall from an accelerating rotarod and for generalized activity using open field activity chambers. All behavioral tests were performed by individuals blinded to treatment.

Results

Rotarod Performance

At the highest dose administered (2.5×10¹¹ vg), AAV/Olig001-ASPA rescued progressively deteriorating balance, grip strength and/or motor coordination as measured by rotarod performance in nur7 mice to a level indistinguishable from age-matched wild type animals and highly significantly improved over sham nur7 controls. At this dose, increased rotarod performance in AAV/Olig001-ASPA treated animals was significant across the entire study period, as determined by repeat measures ANOVA (p=0.028) and significantly higher at each individual time point as determined by unpaired Students t-test. At the mid-range dose (7.5×10¹⁰ vg), AAV/Olig001-ASPA also promoted significantly improved rotarod performance in nur7 mice at each time point tested, but this improvement was not significant over the entire study period (repeat measures ANOVA p=0.19). At the lowest dose administered (2.5×10¹⁰ vg), AAV/Olig001-ASPA was effective at promoting improved rotarod performance in the last two time points tested only (18 and 22 months). Table 1 provides mean latency to fall measured in seconds for each treatment group (with standard deviation). For each group 12 mice (6 male and 6 female) were tested. Table 2 provides p-values for unpaired t-test comparisons between AAV/Olig001-ASPA treated and sham nur7 mice at each age. Statistically significant improvements were observed in all groups except for mice administered 2.5×10¹⁰ vs. sham treated mice at 10 and 14 weeks.

TABLE 1
Rotarod latency to fall.

| | Mean (SD) latency to fall (seconds) | | | | | |
|---|---|---|---|---|---|---|
| Dose | 10 weeks | | 14 weeks | 18 weeks | 22 weeks | |
| 2.5 × 10¹¹ | 239.83 | (36.7) | 234.89 (32.3) | 217.78 (41.1) | 201.63 | (50.2) |
| 7.5 × 10¹⁰ | 230.278 | (52.1) | 222.67 (64.7) | 199.78 (55.7) | 183.28 | (50.4) |
| 2.5 × 10¹⁰ | 218.25 | (68.6) | 195.89 (97.6) | 171.14 (55.7) | 169.08 | (55.8) |
| Sham | 187.94 | (41.8) | 148.39 (39.5) | 119.39 (27.2) | 96.06 | (23.5) |
| Wild type | 232.95 | (34.5) | 234.12 (37.8) | 227.63 (31.1) | 208.78 | (55.5) |

TABLE 2
P values for difference in rotarod latency between AAV/Olig001-
ASPA treated mice and sham treated mice.

| | P value for unpaired t-test | | | |
|---|---|---|---|---|
| Dose | 10 weeks | 14 weeks | 18 weeks | 22 weeks |
| 2.5 × 10¹¹ vs. sham | 0.00385137 | 6.5919E−06 | 6.0627E−07 | 1.2282E−06 |
| 7.5 × 10¹⁰ vs. sham | 0.03906939 | 0.00272419 | 0.00018 | 1.8767E−05 |
| 2.5 × 10¹⁰ vs. sham | 0.2046576 | 0.13253963 | 0.00849161 | 0.00039049 |

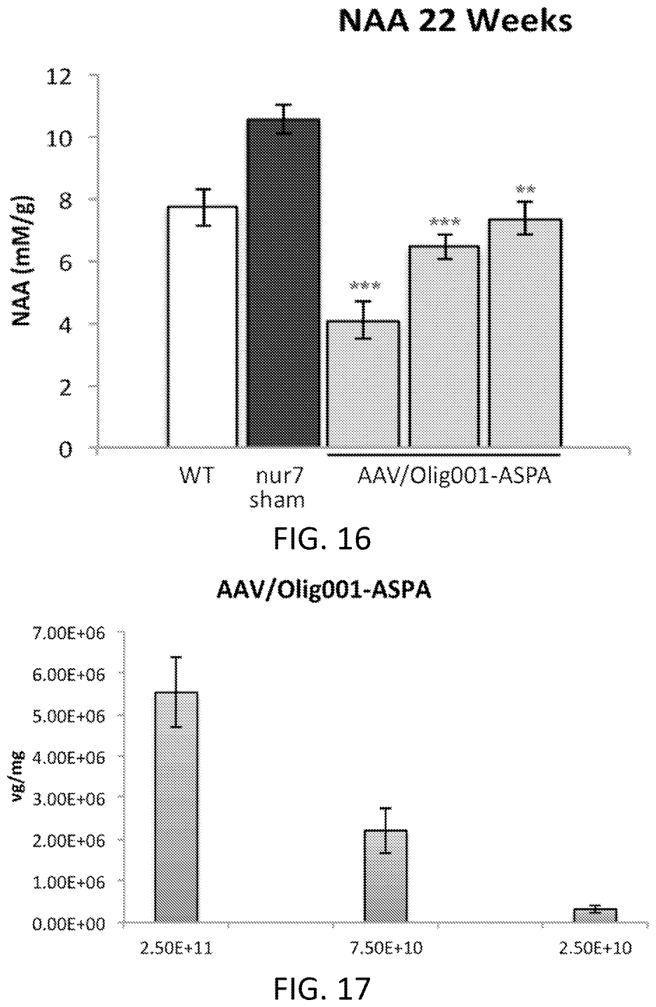

FIG. 14 shows plotted rotarod mean latency to fall over the course of in-life study period for each AAV/Olig001-ASPA nur7 dose cohort, sham nur7, and naïve wild type controls. Latency to fall was increased in all 3 dose cohorts, with the highest dose being significant over the whole study period by repeat measures ANOVA (*).

Open Field Activity

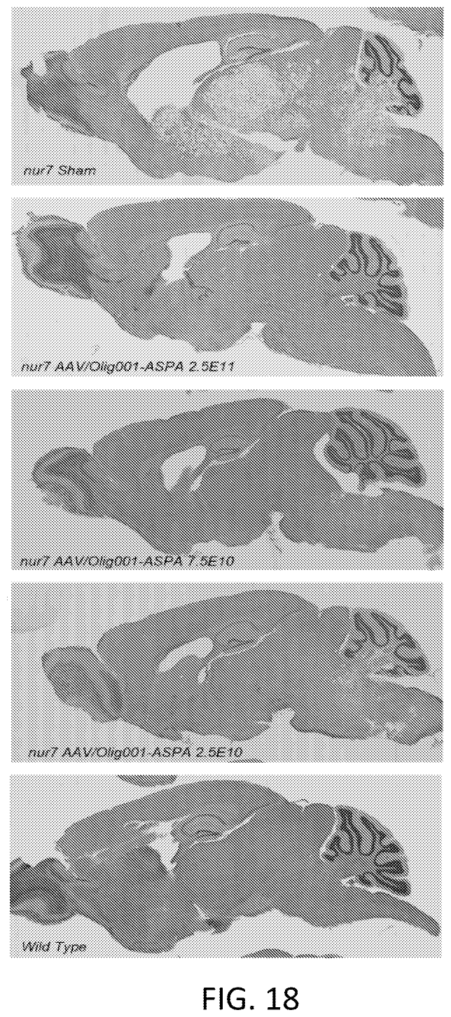

At each age for which rotarod was conducted, animals were also assessed for generalized motor function in open field activity chambers (FIG. 15). Animals were given single 20-minute sessions each time, and total distance travelled per session was recorded. Relative to age-matched wild type animals, sham nur7 mice exhibited significantly hyperactive at all ages, particularly over the latter time points. At 22 weeks of age, sham nur7 animals presented with a significant 3-fold increase in activity (distance travelled; p=0.0202) over wild type. By contrast, the 2.5×10¹¹ dose of AAV/Olig001-ASPA resulted in normalized activity levels in nur7 mice that were statistically significant relative to sham controls (p=0.0312) and indistinguishable from age-matched wild type. The lower 7.5×10¹⁰ dose of AAV/Olig001-ASPA resulted in activity patterns that more closely resembled wild type than sham nur7 patterns, but were just below the threshold for statistical significance versus sham at 22 weeks of age (p=0.1181). The lowest)(2.5×10¹⁰ dose of AAV/Olig001-ASPA did not significantly normalize pathological hyper-activity and more closely resembled sham nur7 controls than wild type references.

Assessment of open field activity in these same animals demonstrated a dose-dependent normalization of hyperactivity in AAV/Olig001-ASPA treated nur7 animals. The data are presented as mean+/−sem with n=6 animals per group.

NAA Accumulation and Vector Genome (Vg) Copy Number

Following rotarod testing at 22 weeks, mice were sacrificed, and brain tissue was isolated. One hemisphere of each brain was processed for the HPLC analysis of NAA, and the remaining hemisphere processed for analysis of vector genome (vg) copy number by quantitative PCR.

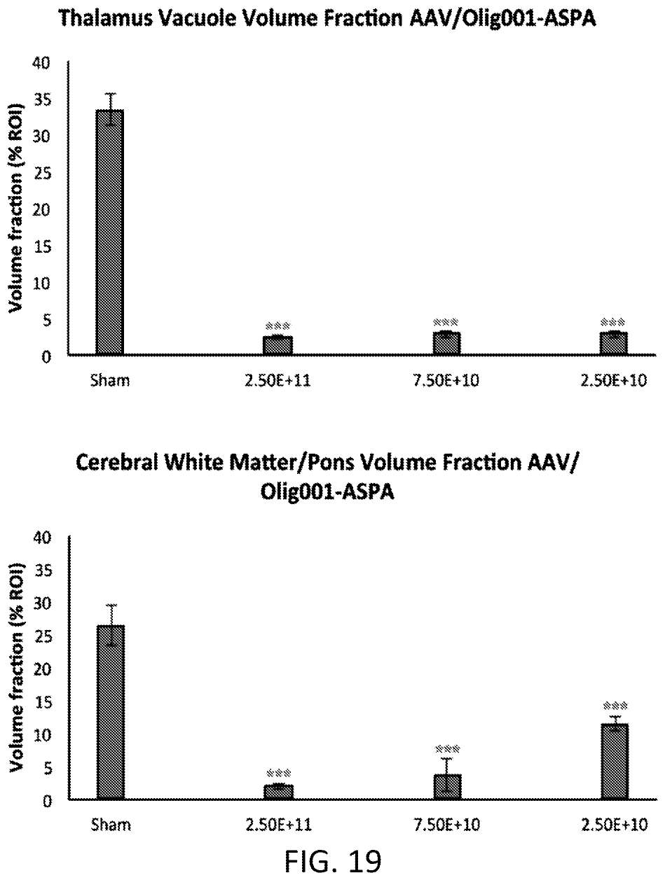

Sham saline treated nur7 mouse brains contained typically elevated NAA as expected from loss of ASPA function (FIG. 16). A dose responsive reduction in pathologically elevated NAA was observed in AAV/Olig001-ASPA treated cohorts, with the highest 2.5×10¹¹ dose resulting in a highly significant 2.6-fold reduction (p=5.06×10⁻⁶), the mid 7.5×10¹⁰ dose a 1.6-fold reduction (p=5.17×10⁻⁵), and the lowest 2.5×10¹⁰ dose a 1.4-fold reduction (p=0.001). NAA in nur7 brains treated with the highest dose of AAV/Olig001-ASPA was in fact significantly lower than in age-matched wild type brains (p=0.0012).

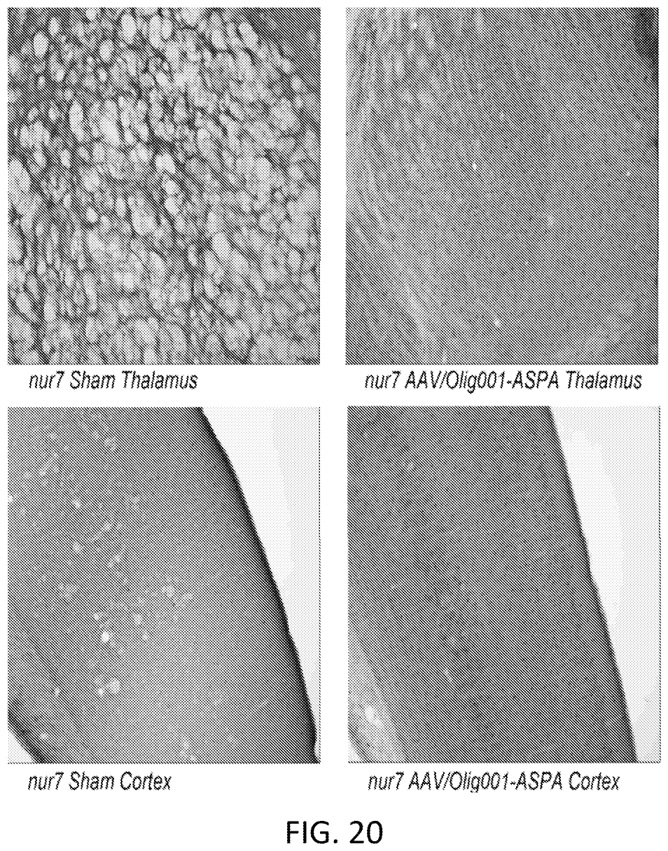

The hemispheres remaining from brains analyzed for NAA were used to quantify vector genome (vg) copy number by quantitative PCR using a custom TaqMan probe/primer set targeted to the bovine growth hormone (BGH) polyadenylation sequence of the recombinant AAV/Olig001-ASPA expression cassette. Total DNA content of hemispheres was isolated using commercially available DNA purification columns and kits (Qiagen) and samples of DNA thus generated run against a purified plasmid standard curve to generate vg/wet tissue weight for each sample. VG/mg of tissue values generated reflected the dose of AAV/Olig001-ASPA administered (FIG. 17), consistent with the response of NAA to vector dose.

Vacuolation Analysis

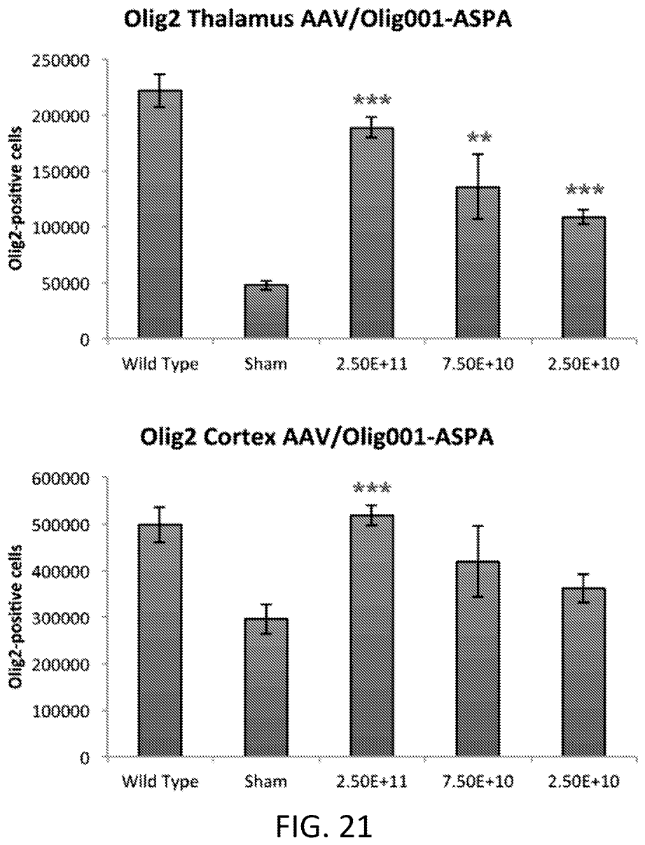

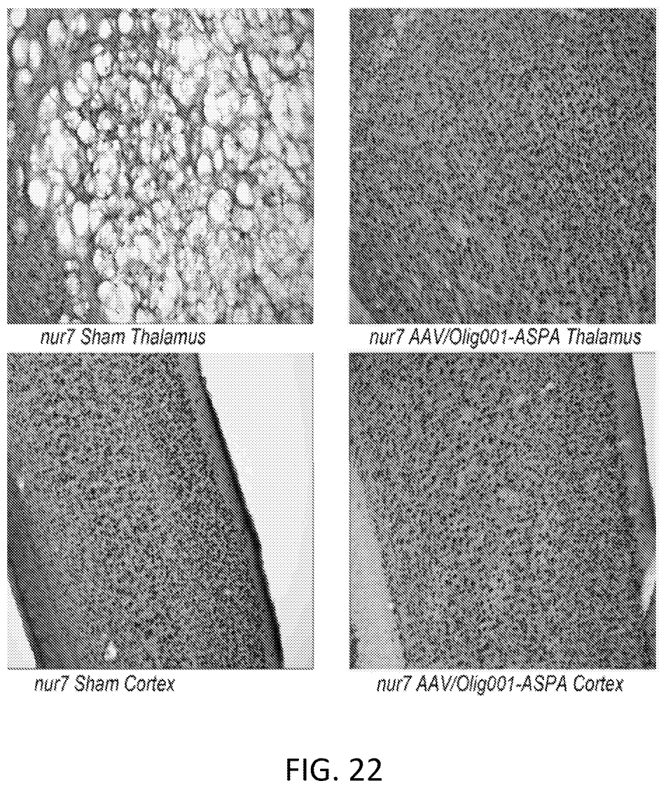

Brains of nur7 mice treated with AAV/Olig001-ASPA were analyzed by unbiased stereology to quantify vacuole volume fraction in the thalamus and cerebellar white matter/pons as a function of vector dose (FIG. 18). The areas within each region of interest occupied by empty space were defined as vacuoles and presented as a percentage of overall region of interest volume. At each dose, AAV/Olig001-ASPA treatment resulted in full rescue of thalamic vacuolation as shown by highly significant reductions in thalamic vacuole volume fractions (2.5×10¹¹, p=4.6×10⁻⁸; 7.5×10¹⁰, p=6.4×10⁻⁸; and 2.5×10¹⁰, p=6.2×10⁻⁸) as compared to sham treated mice (FIG. 19). Vacuolation in cerebellar white matter/pons was also significantly rescued at all doses (2.5×10¹¹, p=1.3×10⁻⁵; 7.5×10¹⁰, p=2.5×10⁻⁵; and 2.5×10¹⁰, p=0.0009) as compared to sham treated mice, but the degree of rescue was proportional to dose of vector administered. The lowest 2.5×10¹⁰ dose cohort presented with a vacuole volume fraction that was significantly increased over that for the highest 2.5×10¹¹ dose (p=5.74×10⁻⁶) while still significantly less than vacuole volume fraction in sham treated controls (p=0.0009) (FIG. 19).

Oligodendrocyte Recovery

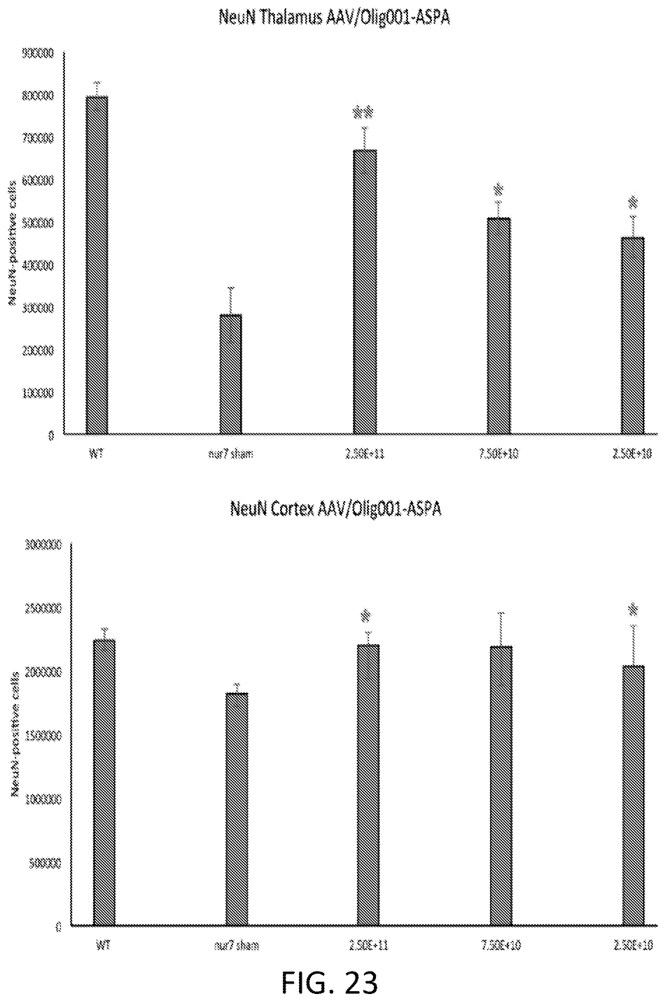

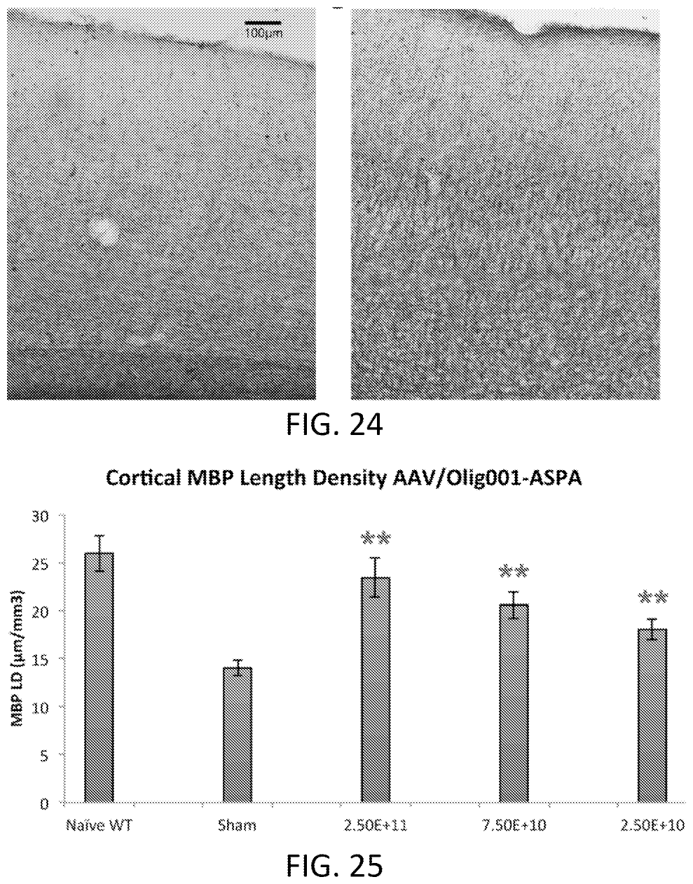

The same brains analyzed for vacuolation were processed for Olig2 immunohistochemistry to identify oligodendrocytes. Both the thalamus and cortex were sampled for Olig2-positive cells by unbiased stereology to identify significant differences in resident white matter producing cells in areas both affected and unaffected by vacuolation, respectively (FIG. 20). Sham nur7 brains presented a massive 4.6-fold loss of Olig2-positive cells relative to age-matched wild type brains, representing only 21% of the normal wild type content (p=4.9×10⁻⁷). Olig2 counts in the thalamus of AAV/Olig001-ASPA treated nur7 mice and sham treated nur7 mice (FIG. 21) revealed a significant increase in oligodendrocytes in all three AAV/Olig001-ASPA treated nur7 cohorts relative to sham controls (2.5×10¹¹ vg, p=6.75×10⁻⁸; 7.7×10¹⁰ vg, p=0.026; 2.3×10¹⁰ vg, p=3.18×10⁻⁵). Olig2 loss in cortical areas was less dramatic but significant (1.7-fold reduction in sham treated nur7 mice vs. wild type mice; p=0.0025). The Olig2 content of the cortex (FIG. 21) of 2.5×10¹¹ vg treated nur7 brains was also significantly increased relative to sham treated nur7 control mice (p=0.0002), but the two lower dose cohort brains were not.

Neuronal Recovery

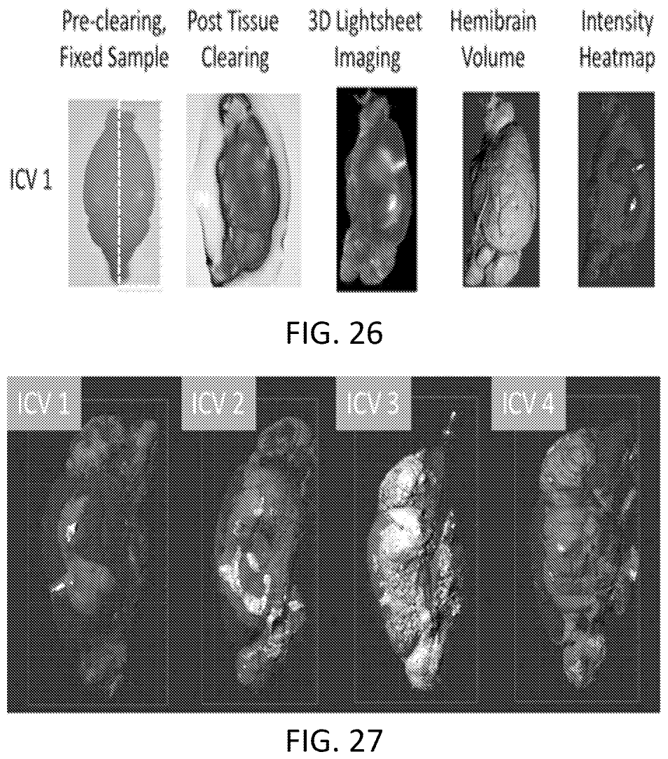

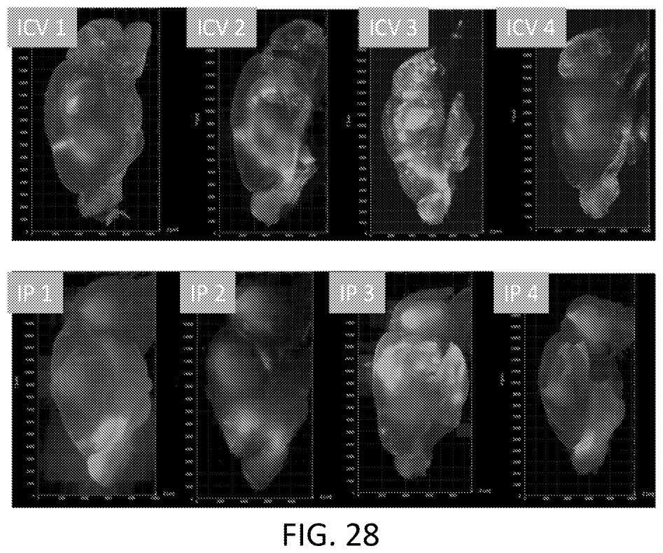

The thalamus and cortex were scored for NeuN-positive neurons in the same 22-week old brains used for Olig2 staining (FIG. 22). Sham treated nur7 animals presented with numbers of thalamic neurons that were ˜35% of age-matched wild type animal values (p=2.8×10⁻⁵) (FIG. 23). Nur7 mice treated with 2.5×10¹¹ AAV/Olig001-ASPA contained numbers of thalamic neurons that were increased 2.3-fold over sham treated control mice (p=0.0009) and about 84% of thalamic neurons observed in wild type mice. At the two lower doses, 7.5×10¹⁰ and 2.5×10¹⁰, AAV/Olig001-ASPA promoted increased thalamic NeuN-positive cells that were 1.8 and 1.6-fold increased over sham treated control mice, respectively (p=0.012; p=0.042). In the cortex (motor and somatosensory), neuronal loss in sham treated nur7 mouse brains relative to age-matched wild type mouse brains was less profound, but significant. Cortices from sham treated mice contained about 80% of NeuN-positive cells observed in wild type mice, representing a 1.2-fold reduction (p=0.005). Nur7 mice treated with 2.5×10¹¹ AAV/Olig001-ASPA contained numbers of cortical neurons that were ˜98% of the cortical neurons observed in wild type mice, and 1.2-fold increased the number of cortical neurons observed in sham treated nur7 mice (p=0.013). Successive doses of AAV/Olig001-ASPA resulted in a stable 1.2-fold increase in cortical neurons relative to sham treated. For the 7.5×10¹⁰ dose, a high variance in sampled data rendered this increase nonsignificant (p=0.113). At the lowest 2.5×10¹⁰ dose, AAV/Olig001-ASPA treated mice maintained a significant 1.2-fold increase in cortical neurons over sham treated controls (p=0.05).

Improved Myelination

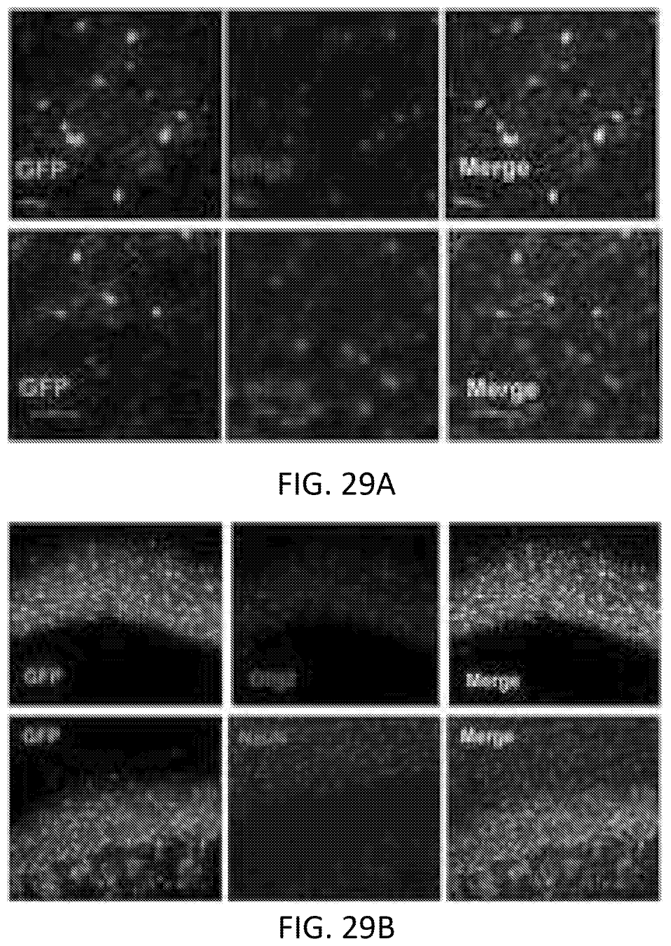

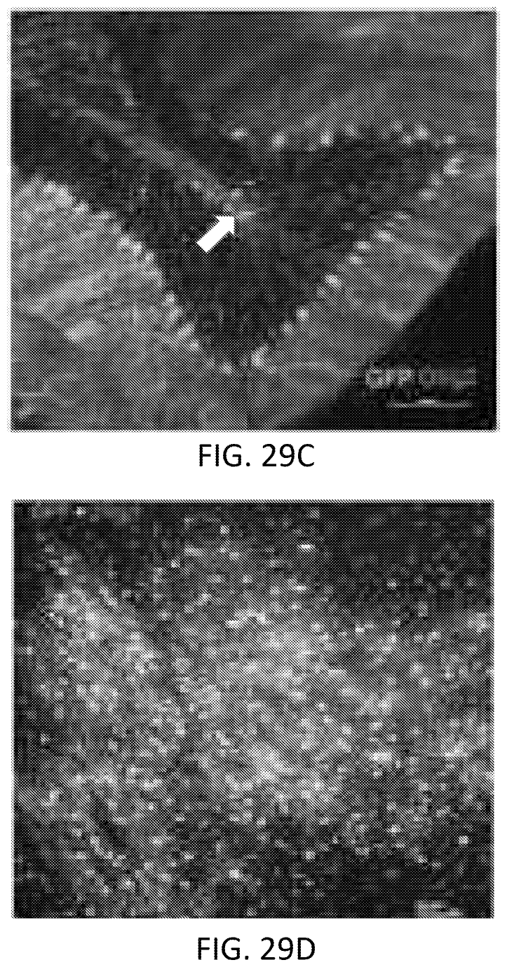

Unbiased stereology was used to quantify cortical myelin basic protein-positive fiber length density (MBP-LD) throughout the cortex of sham treated and AAV/Olig001-ASPA treated 22-week old nur7 brains to provide an index of the degree of recovery of myelination following treatment with AAV/Olig001-ASPA. The motor and somatosensory cortex was sampled for MBP-positive fibers using a computer-generated probe to score for isotropic probe fiber interactions in the 3-dimensional tissue space, and the sum total MBP-positive fiber length within cortices divided by volume of tissue sampled to give a final MBP length density (μm fibers per mm³) (FIG. 24). When compared to age-matched wild type brains, Sham nur7 brains presented a highly significant 2-fold reduction in cortical MBPLD (p=0.0001). Treatment with AAV/Olig001-ASPA at all three doses resulted in significant increases in cortical MBP-LD relative to sham controls, with degree of improvement proportional to dose (2.5×10¹¹ p=0.0014; 7.5×10¹⁰ p=0.003; 2.5×10¹⁰ p=0.016). Sham treated and AAV/Olig001-ASPA treated nur7 mouse brains were stained with anti-myelin basic protein (MBP) (FIG. 25).

These data demonstrate that AAV/Olig001-ASPA treatment of a mouse model of Canavan disease improves balance, grip strength and/or motor coordination, motor function, reduces the amount of NAA present in the brain, reduces vacuolation of the brain, increases the number of Olig2 and NeuN positive cells and restores myelination.

### Example 6: CLARITY-Aided Biodistribution for Canavan Gene Therapy

The biodistribution of an oligodendrocyte-tropic rAAV vector (Olig001) with a Green Fluorescent Protein (GFP) transgene in Canavan disease-phenotype presenting Nur7 mouse brains was evaluated using a three-dimensional (3D) tissue clearing and imaging method. This allowed for a global representation and volumetric measurement of the vector biodistribution within Nur7 mice hemibrains administered via alternate routes of administration (ROA). Intracerebroventricular (ICV) and intraparenchymal (IP) ROAs were compared for biodistribution efficacy and this method was used to supplement conventional stereology data obtained from traditional, two-dimensional (2D) histological evaluation.

This example demonstrates the applicability of the 3D method, and its significance in assessing AAV/Olig001-GFP biodistribution, in adult murine hemibrains of Canavan disease mouse models. Results are presented as visual qualitative and quantitative representations of 3D cleared brain images of lightsheet microscopy data and tabulated parameters of biodistribution estimations.

Sample Preparation and Imaging

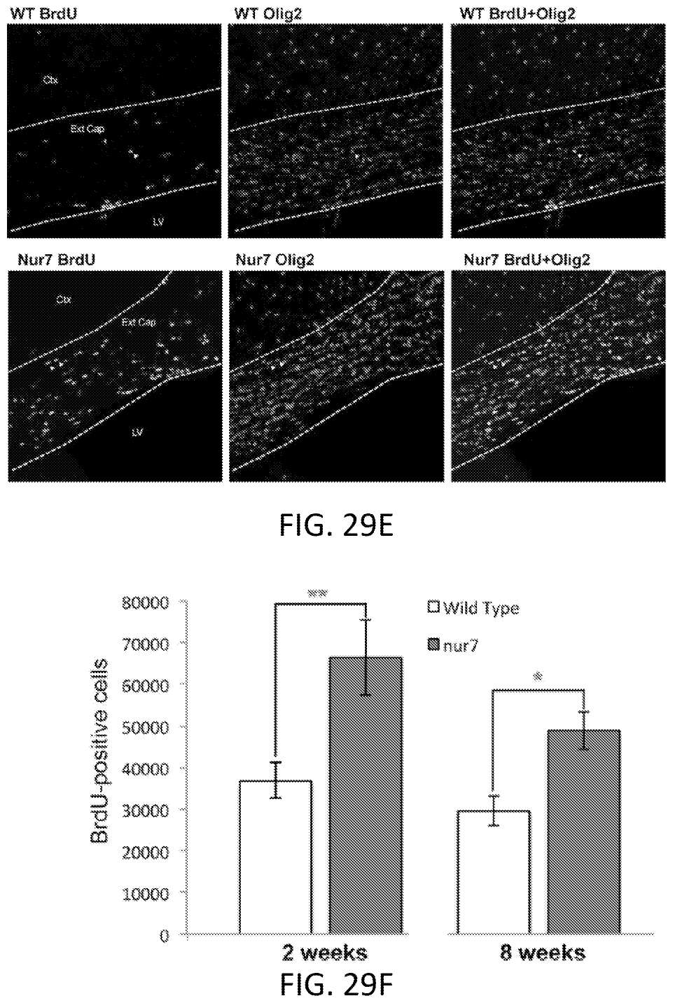

Four adult mice per ROA (eight total) received 5×10¹¹ vector genome (vg)/animal at 6 weeks of age and sacrificed two weeks post dosing. PFA-fixed brains were received and prepared for 3D tissue clearing and volumetric lightsheet microscopy imaging. Each brain was sagittally bisected and the right hemispheres were subjected to tissue clearing using CLARITY (Chung et al., Nature, 2013). Each sample was prepared identically with hydrogel embedding and polymerization followed by electrophoretic tissue clearing using a commercial device (X-Clarity, Logos Biosystems) utilizing commercially available reagents (Logos Biosystems). Macroscopic micrographs at major steps during the sample handling were acquired to document sample conditions (FIG. 26).

Full, 3D microscopy imaging of each cleared hemibrain was performed using a Zeiss Z.1 lightsheet microscope, utilizing a 5× magnification objective and tiling-based acquisition covering the entirety of each hemibrain. Imaging parameters were adjusted to detect the GFP expression and kept constant across all samples to ensure consistency and enable relative comparisons across samples. All samples were processed and imaged under identical conditions from tissue clearing through image acquisition and analysis.

Image Processing and Analyses

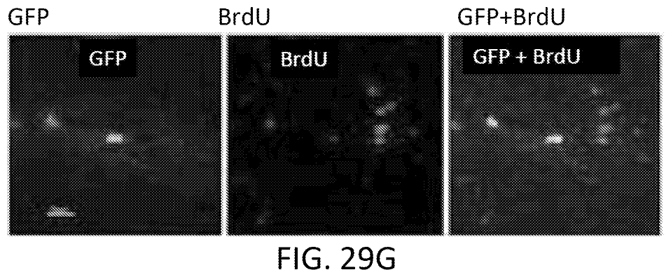

The raw dataset was preprocessed and reconstructed into a full, seamless 3D image using an in-house custom designed algorithm for each hemibrain. Final images each contained one hemibrain and were imported into a commercial 3D image processing and analysis program (Imaris, Bitplane) for a global, quantitative biodistribution analysis. First, a global average and median (GFP) signal value within the full hemibrain volume was calculated. Furthermore, two GFP intensity thresholds were chosen to designate “low” or “high” GFP expression (FIG. 27). These thresholds were then kept constant across all samples for consistency. The volumes of these classified intensity regions were then determined and compared to the full hemibrain volume to give rise to the “vol % high/low expression” (Table 3).

Results

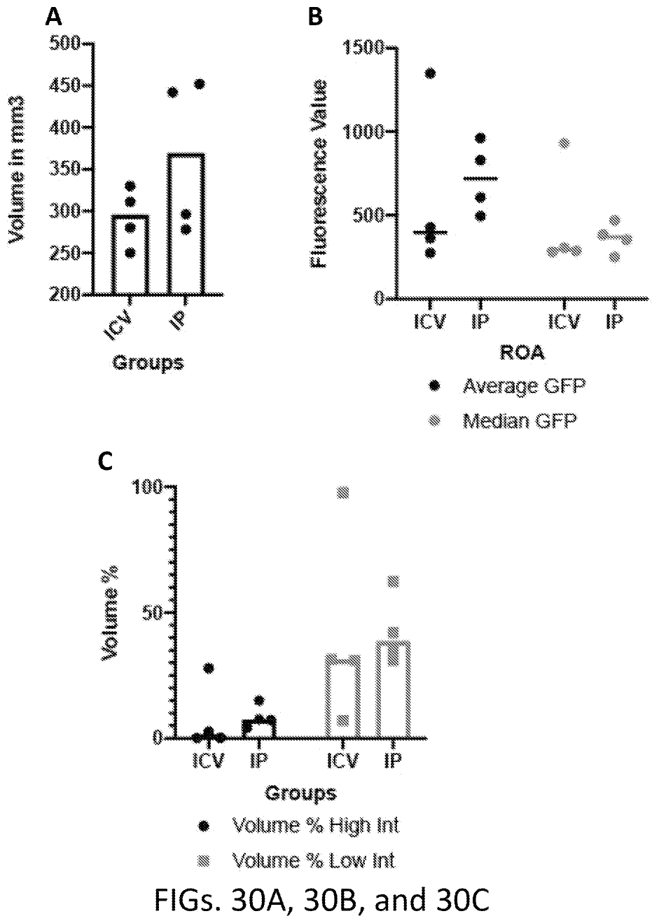

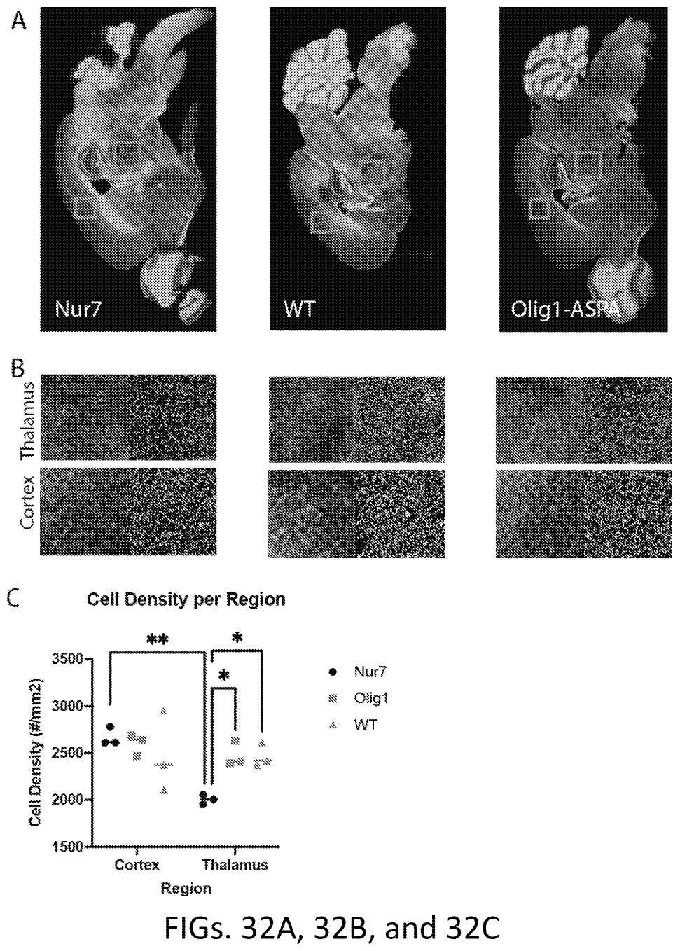

The macroscopic micrographs and the complete 3D imaging of each hemibrain revealed variable biodistribution patterns of GFP expression across the two ROAs (IP vs. ICV; FIG. 28 and FIG. 30). Additionally, cell-type tropism was evaluated by visual assessment of cell morphology and their spatial location determined. While these biodistribution patterns differed across samples depending on the extent of vector spread, similarities in subregional transduction patterns remained consistent across samples such as the high expression in Purkinje cells in the cerebellum. The quantification of ‘low’ and ‘high’ GFP expression along with global intensities were then calculated and tabulated for each hemibrain (Table 3). Consistent with stereological assessment in the prior Example, cleared hemibrains displayed superior vector spread within the subcortical white matter following ICV injections, which is a critical region for Canavan disease. Additionally, while IP injection resulted in subregions of high GFP intensity, the majority of these subregions were concentrated around injection sites, supporting the conclusion obtained from stereological assessment.

TABLE 3
Quantification for 4 ICV-injected hemibrains.

| | Average | Median | Volume in | Vol % high | | Vol % low | |
|---|---|---|---|---|---|---|---|
| | Intensity | Intensity | mm³ | expression | | expression | |
| ICV1 | 276 | 282 | 280 | 0.14% | [0.39 mm³] | 7.04% | [19.72 mm³] |
| ICV2 | 428 | 287 | 330 | 2.69% | [8.87 mm³] | 30.94% | [102.1 mm³] |
| ICV3 | 1348 | 931 | 250 | 27.86% | [69.64 mm³] | 97.82% | [244.54 mm³] |
| ICV4 | 363 | 305 | 311 | 0.30% | [0.92 mm³] | 31.34% | [97.47 mm³] |

Conclusion and Significance

Volumetric imaging of intact, tissue clarified, murine brains provide a more comprehensive and holistic assessment of AAV/Olig001 biodistribution. The custom algorithms to enable full acquisition and quantification of the distribution supports higher-resolution quantification obtained from stereology methods. Assessment of organ-level imaging provides global evaluation of this biodistribution retaining 3D spatial structural and regional connectivity. Finally, the digital compilation of various ROAs can be used to generate a digital ‘library’ to be used for future references when performing additional assessments into AAV/Olig001 ROAs to assess optimal transduction efficiencies and cell-type specific tropism.

### Example 7: CLARITY-Based Volumetric Assessment of AAV Biodistribution and Pharmacodynamic Effect

In this example, the CLARITY tissue clearing technique described in Example 6 above was utilized to assess and demonstrate the global and local transgene-mediated pharmacological effect of reversal in demyelination after injection with AAV/Olig001-ASPA in nur7 mouse brains.

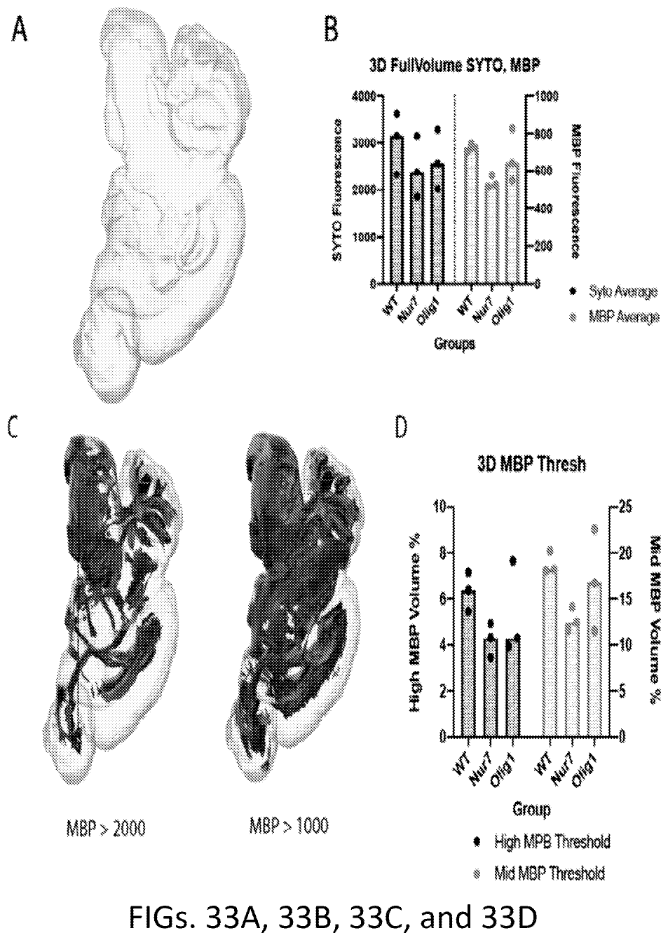

Briefly, nur7 mice were divided into two groups and administered with AAV/Olig001-ASPA (“Olig1” or “Olig1-ASPA”) or saline (“Nur7”) via the ICV or IP route in the manner described above. Brains of the two groups of mice were then analyzed to quantify vacuole volume fraction in the thalamus and cerebellar white matter/pons in the manner described above. Brains of wild type mice (“WT”) as a control group were also analyzed. The results are shown in FIG. 31. More specifically, the arrowheads in FIG. 31B indicate that the thalamic region of nur7 mice exhibited visible vacuolation, which was non-existent in WT and almost fully rescued in Olig1-ASPA treated tissue. In addition, as shown in FIG. 31C, after one day of passive clearing, nur7 mouse tissues reached higher transparency than both WT and Olig1-ASPA treated tissues. These results demonstrate that AAV/Olig001-ASPA treatment reduced vacuolation of the brain and restored myelination in the nur7 mice.

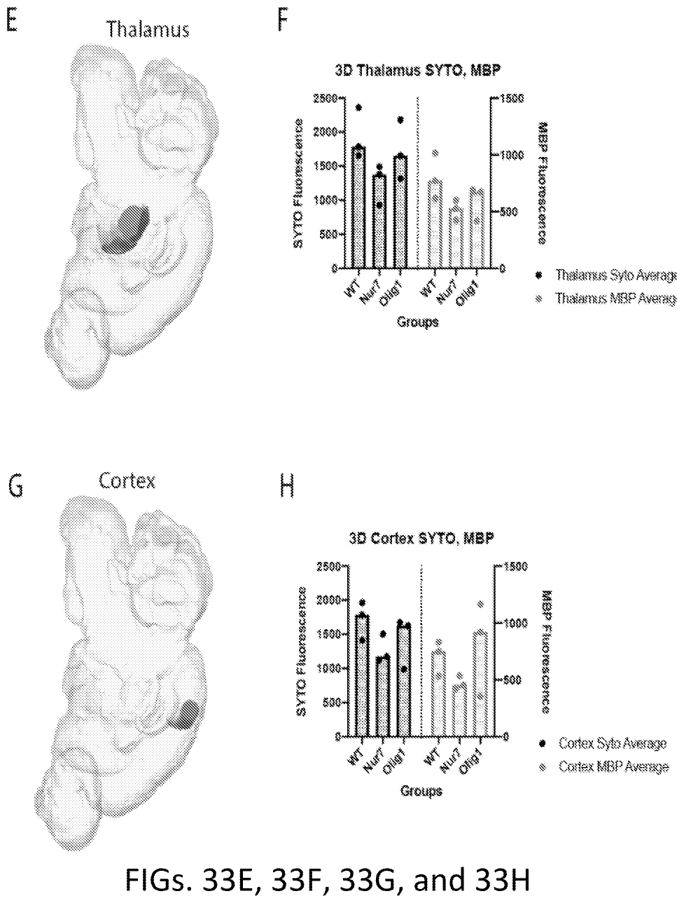

Cell counting analysis was also carried out in extracted 2D single slices of 3D images from all three groups with similar anatomical orientation (FIG. 32A). As shown in FIGS. 32B and 32C, although average nuclei density (counts normalized by segmentation area) showed overall little difference in cell density within the cortical region, mice of the Nur7 group had a significantly lower overall nuclei density/nuclei area in the thalamic region. In contrast, the Olig1-ASPA group and the WT group appeared to have similar overall nuclei densities or nuclei areas in the thalamic regions. These results demonstrate that AAV/Olig001-ASPA treatment of the nur7 mice maintained or increased the number of cells in the thalamic region to a level close to that seen in the WT group.

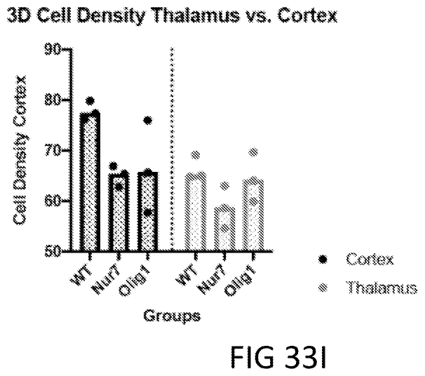

The brains analyzed for vacuolation were processed for immunofluorescent staining of MBP to identify oligodendrocytes in the manner described above. To that end, 3D volumetric analysis was carried out to examine pharmacodynamic treatment effect. The full 3D volume of a 2-mm tissue slice was determined and the average fluorescence intensity calculated for SYTO (nuclear marker), as well as for MBP. It was found that tissues from mice of the Nur7 group exhibited a lower average MBP fluorescence value. In contrast, the Olig1-ASPA treated group had increased overall MBP signals, which were almost to the levels of the WT group (FIG. 33B).

Additional 3D volumetric analysis was carried out, where the MBP volume was calculated via a signal threshold. The threshold was performed either more restrictively with a threshold set at fluorescence value of over 2000 (FIG. 33C, left panel), or more inclusively with a threshold at 1000 (FIG. 33C, right panel). In both cases, MBP deficits were observed in the mice of the Nur7 group (FIG. 33D). In contrast, an increase in the MBP volume was clearly seen in the Olig1-ASPA group and particularly when using the lower threshold, where the overall MBP volume value approached the level of the WT group (FIG. 33D).

Region-based analyses were performed in 3D in the thalamic region. A manual segmentation of a portion of the region was shown in FIG. 33E. Average fluorescence intensities within this region for both nuclei (SYTO) and myelin (MBP) markers were shown in FIG. 33F. It was found that the SYTO and MBP levels of the Olig1-ASPA group almost reached to the levels of the WT group. In contrast, the Nur7 samples exhibited lower average fluorescence values in both markers. Region-based analyses were also performed on a portion of the cortex. Shown in FIGS. 33G and 33H were average fluorescence intensity levels within this cortical region for both nuclei (SYTO) and myelin (MBP) markers. The overall trends were similar to those shown in FIG. 33F. 3D cell concentrations (nuclei per 100 um²) in the cortex and the thalamic region were also obtained. As shown in FIG. 33I, the overall nuclei concentrations in both regions in the mice of the Nur7 group were lower. In contrast, the 3D cell concentrations in thalamic regions of the mice of the Olig1-ASPA group exhibited levels that were close to the levels of the WT group.

These results demonstrate that administration of AAV/Olig001-ASPA rescued or reversed demyelination and cell loss in nur7 mouse brains.

### EQUIVALENTS

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, text books, and the like, and the references cited therein, to the extent that they are not already, are hereby incorporated herein by reference in their entirety.

TABLE 4
SEQUENCES

| SEQ ID NO: | Description | Sequence |
|---|---|---|
| SEQ ID NO: 1 | Codon- | ATGACCTCCTGTCATATAGCCGAGGAGCACATCCAGAAAGTGGCCATT |
| | optimized | TTCGGCGGGACACATGGGAACGAGCTGACTGGCGTTTTCCTGGTCAAG |
| | ASPA- | CACTGGCTCGAAAATGGCGCGGAAATTCAGAGAACGGGCCTGGAGGTC |
| | original | AAACCTTTTATTACTAACCCCCGCGCGGTGAAGAAATGTACCCGGTAC |
| | | ATCGACTGCGATCTTAACCGAATCTTTGATCTGGAAAATCTGGGAAAA |
| | | AAAATGAGCGAGGACCTGCCCTACGAAGTCCGCAGAGCACAGGAGATT |
| | | AATCATCTCTTCGGACCCAAGGACTCCGAGGACAGCTACGATATCATC |
| | | TTCGACTTGCACAATACTACTTCCAATATGGGATGTACCTTGATACTG |
| | | GAGGACTCACGAAATAACTTCTTGATTCAGATGTTCCATTAGATCAAA |
| | | ACCTCTCTCGCTCCTCTCCCTTGCTACGTATATTTGATCGAGCACCCT |
| | | AGTCTGAAATATGCCACTACACGAAGCATAGCTAAGTATCCCGTTGGT |
| | | ATTGAGGTGGGCCCCCAGCCCCAGGGAGTGCTGCGGGCTGACATCCTT |
| | | GACCAGATGAGAAAAATGATCAAACACGCCCTTGACTTCATCCACCAC |
| | | TTTAATGAAGGCAAAGAGTTTCCTCCCTGTGCCATAGAGGTGTATAAA |
| | | ATCATCGAAAAAGTTGACTATCCACGGGATGAGAACGGCGAGATCGCT |
| | | GCCATCATCCATCCCAATTTGCAAGATCAGGATTGGAAACCTTTGCAC |
| | | CCAGGCGACCCTATGTTCCTGACATTGGATGGCAAGACCATACCCCTG |
| | | GGTGGTGATTGCACTGTGTACCCAGTTTTCGTAAACGAGGCAGCGTAC |
| | | TATGAAAAGAAAGAGGCATTTGCAAAAACCACTAAGTTGACACTGAAT |
| | | GCCAAGAGCATTAGATGCTGTCTTCATTAA |
| SEQ ID NO: 2 | Codon- | ATGACCTCCTGTCATATAGCCGAGGAGCACATCCAGAAAGTGGCCATT |
| | optimized | TTCGGCGGGACACACGGAAACGAACTTACAGGAGTGTTTCTGGTGAAA |
| | ASPA-new | CACTGGCTTGAAAATGGTGCGGAGATCCAAAGGACCGGCCTGGAGGTC |
| | | AAACCTTTTATTACAAATCCCCGGGCGGTCAAGAAGTGCACACGGTAC |
| | | ATTGATTGTGATCTTAATCGCATATTCGACCTGGAGAACCTTGGGAAG |
| | | AAAATGTCTGAAGATCTGCCCTACGAAGTGAGGCGAGCACAAGAGATA |
| | | AACCACCTGTTCGGACCGAAAGACAGTGAAGACTCCTATGACATCATT |
| | | TTCGACCTGCACAACACTACGAGTAACATGGGGTGTACCCTGATCCTC |
| | | GAAGACTCCCGAAACAATTTCCTGATACAGATGTTTCATTAGATCAAA |
| | | ACTAGTCTGGCCCCTCTCCCCTGCTACGTTTATCTGATCGAACACCCT |
| | | TCTCTCAAATACGCTACCACCCGCTCTATTGCTAAGTACCCCGTCGGG |
| | | ATCGAGGTCGGCCCACAACCTCAAGGTGTGCTCCGGGCCGATATTTTG |
| | | GACCAGATGAGAAAGATGATTAAACACGCTCTCGACTTCATTCACCAC |
| | | TTTAACGAGGGGAAGGAATTTCCCCCTTGTGCCATCGAGGTTTATAAG |
| | | ATTATCGAGAAGGTGGACTACCCAAGAGACGAAAACGGGGAGATAGCT |
| | | GCCATCATCCACCCTAATTTGCAAGATCAGGACTGGAAGCCCCTGCAC |
| | | CCAGGAGACCCCATGTTTCTGACCTTGGATGGAAAGACGATCCCCCTG |
| | | GGCGGTGATTGTACAGTGTACCCAGTCTTTGTCAACGAGGCCGCTTAC |
| | | TATGAGAAAAAGGAGGCTTTTGCAAAGACAACAAAGCTCACTTTGAAT |
| | | GCAAAGTCCATCAGGTGCTGTCTGCACTAA |
| SEQ ID NO: 3 | Nucleotide | ATGACTTCTTGTCACATTGCTGAAGAACATATACAAAAGGTTGCTATC |
| | sequence | TTTGGAGGAACCCATGGGAATGAGCTAACCGGAGTATTTCTGGTTAAG |
| | encoding | CATTGGCTAGAGAATGGCGCTGAGATTCAGAGAACAGGGCTGGAGGTA |
| | wild type | AAACCATTTATTACTAACCCCAGAGCAGTGAAGAAGTGTACCAGATAT |
| | ASPA | ATTGACTGTGACCTGAATCGCATTTTTGACCTTGAAAATCTTGGCAAA |
| | (NM_000049.4) | AAAATGTCAGAAGATTTGCCATATGAAGTGAGAAGGGCTCAAGAAATA |
| | | AATCATTTATTTGGTCCAAAAGACAGTGAAGATTCCTATGACATTATT |
| | | TTTGACCTTCACAACACCACCTCTAACATGGGGTGCACTCTTATTCTT |
| | | GAGGATTCCAGGAATAACTTTTTAATTCAGATGTTTCATTAGATTAAG |
| | | ACTTCTCTGGCTCCACTACCCTGCTACGTTTATCTGATTGAGCATCCT |
| | | TCCCTCAAATATGCGACCACTCGTTCCATAGCCAAGTATCCTGTGGGT |
| | | ATAGAAGTTGGTCCTCAGCCTCAAGGGGTTCTGAGAGCTGATATCTTG |
| | | GATCAAATGAGAAAAATGATTAAACATGCTCTTGATTTTATACATCAT |
| | | TTCAATGAAGGAAAAGAATTTCCTCCCTGCGCCATTGAGGTCTATAAA |
| | | ATTATAGAGAAAGTTGATTACCCCCGGGATGAAAATGGAGAAATTGCT |
| | | GCTATCATCCATCCTAATCTGCAGGATCAAGACTGGAAACCACTGCAT |
| | | CCTGGGGATCCCATGTTTTTAACTCTTGATGGGAAGACGATCCCACTG |
| | | GGCGGAGACTGTACCGTGTACCCCGTGTTTGTGAATGAGGCCGCATAT |
| | | TACGAAAAGAAAGAAGCTTTTGCAAAGACAACTAAACTAACGCTCAAT |
| | | GCAAAAAGTATTCGCTGCTGTTTACATTAG |
| SEQ ID NO: 4 | Amino acid | MTSCHIAEEHIQKVAIFGGTHGNELTGVFLVKHWLENGAEIQRTGLEV |
| | sequence | KPFITNPRAVKKCTRYIDCDLNRIFDLENLGKKMSEDLPYEVRRAQEI |
| | of human | NHLFGPKDSEDSYDIIFDLHNTTSNMGCTLILEDSRNNFLIQMFHYIK |
| | wild type | TSLAPLPCYVYLIEHPSLKYATTRSIAKYPVGIEVGPQPQGVLRADIL |
| | ASPA | DQMRKMIKHALDFIHHFNEGKEFPPCAIEVYKIIEKVDYPRDENGEIA |
| | (NP_000040.1) | AIIHPNLQDQDWKPLHPGDPMFLTLDGKTIPLGGDCTVYPVFVNEAAY |
| | | YEKKEAFAKTTKLTLNAKSIRCCLH |
| SEQ ID NO: 5 | 5′ ITR | CTGCGCGCTCGCTCGCTCACTGAGGCCGCCCGGGCAAAGCCCGGGCGT |
| | | CGGGCGACCTTTGGTCGCCCGGCCTCAGTGAGCGAGCGAGCGCGCAGA |
| | | GAGGGAGTGG |
| SEQ ID NO: 6 | CMV | CGTTACATAACTTACGGTAAATGGCCCGCCTGGCTGACCGCCCAACGA |
| | enhancer | CCCCCGCCCATTGACGTCAATAGTAACGCCAATAGGGACTTTCCATTG |
| | | ACGTCAATGGGTGGAGTATTTACGGTAAACTGCCCACTTGGCAGTACA |
| | | TCAAGTGTATCATATGCCAAGTACGCCCCCTATTGACGTCAATGACGG |
| | | TAAATGGCCCGCCTGGCATTTGCCCAGTACATGACCTTATGGGACTTT |
| | | CCTACTTGGCAGTACATCTACGTATTAGTCATCGCTATTACCAT |
| SEQ ID NO: 7 | CBh | TCGAGGTGAGCCCCACGTTCTGCTTCACTCTCCCCATCTCCCCCCCCT |
| | promoter | CCCCACCCCCAATTTTGTATTTATTTATTTTTTAATTATTTTGTGCAG |
| | | CGATGGGGGCGGGGGGGGGGGGGGGGCGCGCGCCAGGCGGGGCGGGGC |
| | | GGGGCGAGGGGCGGGGCGGGGCGAGGCGGAGAGGTGCGGCGGCAGCCA |
| | | ATCAGAGCGGCGCGCTCCGAAAGTTTCCTTTTATGGCGAGGCGGCGGC |
| | | GGCGGCGGCCCTATAAAAAGCGAAGCGCGCGGCGGGCG |
| SEQ ID NO: 8 | CBA exon 1 | GGAGTCGCTGCGCGCTGCCTTCGCCCCGTGCCCCGCTCCGCCGCCGCC |
| | | TCGCGCCGCCCGCCCCGGCTCTGACTGACCGCGT |
| SEQ ID NO: 9 | CBA intron 1 | GTGAGCGGGCGGGACGGCCCTTCTCCTCCGGGCTGTAATTAGC |
| SEQ ID NO: 10 | MVM intron | AAGAGGTAAGGGTTTAAGGGATGGTTGGTTGGTGGGGTATTAATGTTT |
| | | AATTACCTGGAGCACCTGCCTGAAATCACTTTTTTTCAGGTTGG |
| SEQ ID NO: 11 | BGH polyA | CTGTGCCTTCTAGTTGCCAGCCATCTGTTGTTTGCCCCTCCCCCGTGC |
| | | CTTCCTTGACCCTGGAAGGTGCCACTCCCACTGTCCTTTCCTAATAAA |
| | | ATGAGGAAATTGCATCGCATTGTCTGAGTAGGTGTCATTCTATTCTGG |
| | | GGGGTGGGGTGGGGCAGGACAGCAAGGGGGAGGATTGGGAAGACAACA |
| | | GCAGGCATGCTGGGGATGCGGTGGGCTCTATGG |
| SEQ ID NO: 12 | 3′ ITR | TCGCCCGACGCCCGGGCTTTGCCCGGGCGGCCTCAGTGAGCGAGCGAG |
| | | CGCGCAGCTGGCGTAATAGCGAAGAGGCCCGCACCGATCGCCCTTCCC |
| | | AACAGTTGCGCAGCCTG |
| SEQ ID NO: 13 | nucleic | ATGGCTGCCGATGGTTATCTTCCAGATTGGCTCGAGGACACTCTCTCT |
| | acid | GAAGGAATAAGACAGTGGTGGAAGCTCAAACCTGGCCCACCACCACCA |
| | sequence | AAGCCCGCAGAGCGGCATAAGGACGACAGCAGGGGTCTTGTGCTTCCT |
| | for | GGGTACAAGTACCTCGGACCCTTCAACGGACTCGACAAGGGAGAGCCG |
| | Olig001 | GTCAACGAGGCAGACGCCGCGGCCCTCGAGCACGACAAAGCCTACGAC |
| | (BNP61) | CGGCAGCTCGACAGCGGAGACAACCCGTACCTCAAGTACAACCACGCC |
| | capsid | GACGCGGAGTTTCAGGAGCGCCTTAAAGAAGATACGTCTTTTGGGGGC |
| | | AACCTCGGGCGAGCAGTCTTCCAGGCCAAAAAGAGGCTTCTTGAACCT |
| | | CTTGGTCTGGTTGAGGAAGCGGCTAAGACGGCTCCTGGAAAGAAGAGG |
| | | CCTGTAGAGCAGTCTCCTCAGGAACCGGACTCCTCCTCGGGCATCGGC |
| | | AAGACAGGCCAGCAGCCCGCTAAAAAGAGACTCAATTTCGGTCAGACT |
| | | GGCGACACAGAGTCAGTCCCAGACCCTCAACCAATCGGAGAACCTCCC |
| | | GCAGCCCCCTCAGGTGTGGGATCTCTTACAATGGCTTCAGGTGGTGGC |
| | | GCACCAGTGGCAGACAATAACGAAGGTGCCGATGGAGTGGGTAGTTCC |
| | | TCGGGAAATTGGCATTGCGATTCCCAATGGCTGGGGGACAGAGTCATC |
| | | ACCACCAGCACCCGAACCTGGGCCCTGCCCACCTACAACAATCACCTC |
| | | TACAAGCAAATCTCCAACGGGACATCGGGAGGAGCCACCAACGACAAC |
| | | ACCTACTTCGGCTACAGCACCCCCTGGGGGTATTTTGACTTTAACAGA |
| | | TTCCACTGCCACTTTTCACCACGTGACTGGCAGCGACTCATCAACAAC |
| | | AACTGGGGATTCCGGCCCAAGAGACTCAGCTTCAAGCTCTTCAACATC |
| | | CAGGTCAAGGAGGTCACGCAGAATGAAGGCACCAAGACCATCGCCAAT |
| | | AACCTTACCAGCACGGTCCAGGTCTTCACGGACTCGGAGTACCAGCTG |
| | | CCGTACGTTCTCGGCTCTGCCCACCAGGGCTGCCTGCCTCCGTTCCCG |
| | | GCGGACGTGTTCATGATTCCCCAGTACGGCTACCTAACACTCAACAAC |
| | | GGTAGTCAGGCCGTGGGACGCTCCTCCTTCTACTGCCTGGAATACTTT |
| | | CCTTCGCAGATGCTGAGAACCGGCAACAACTTCCAGTTTACTTACACC |
| | | TTCGAGGACGTGCCTTTCCACAGCAGCTACGCCCACAGCCAGAGCTTG |
| | | GACCGGCTGATGAATCCTCTGATTGACCAGTACCTGTACTACTTGTCT |
| | | CGGACTCAAACAACAGGAGGCACGGCAAATACGCAGACTCTGGGCTTC |
| | | AGCCAAGGTGGGCCTAATACAATGGCCAATCAGGCAAAGAACTGGCTG |
| | | CCAGGACCCTGTTACCGCCAACAACGCGTCTCAACGACAACCGGGCAA |
| | | AACAACAATAGCAACTTTGCCTGGACTGCTGGGACCAAATACCATCTG |
| | | AATGGAAGAAATTGATTGGCTAATCCTGGCATCGCTATGGCAACACAC |
| | | AAAGACGACAAGGAGCGTTTTTTTCCCAGTAACGGGATCCTGATTTTT |
| | | GGCAAACAAAATGCTGCCAGAGACAATGCGGATTACAGCGATGTCATG |
| | | CTCACCAGCGAGGAAGAAATCAAAACCACTAACCCTGTGGCTACAGAG |
| | | GAATACGGTATCGTGGCAGATAACTTGCAGCAGCAAAACACGGCTCCT |
| | | CAAATTGGAACTGTCAACAGCCAGGGGGCCTTACCCGGTATGGTTTGG |
| | | CAGAACCGGGACGTGTACCTGCAGGGTCCCATCTGGGCCAAGATTCCT |
| | | CACACGGACGGCAACTTCCACCCGTCTCCGCTGATGGGCGGCTTTGGC |
| | | CTGAAACATCCTCCGCCTCAGATCCTGATCAAGAACACGCCTGTACCT |
| | | GCGGATCCTCCGACCACCTTCAACCAGTCAAAGCTGAACTCTTTCATC |
| | | ACGCAATACAGCACCGGACAGGTCAGCGTGGAAATTGAATGGGAGCTG |
| | | CAGAAGGAAAACAGCAAGCGCTGGAACCCCGAGATCCAGTACACCTCC |
| | | AACTACTACAAATCTACAAGTGTGGACTTTGCTGTTAATACAGAAGGC |
| | | GTGTACTCTGAACCCCACCCCATTGGCACCCGTTACCTCACCCGTCCC |
| | | CTGTAA |
| SEQ ID NO: 14 | Amino acid | MAADGYLPDWLEDTLSEGIRQWWKLKPGPPPPKPAERHKDDSRGLVLP |
| | sequence | GYKYLGPFNGLDKGEPVNEADAAALEHDKAYDRQLDSGDNPYLKYNHA |
| | for | DAEFQERLKEDTSFGGNLGRAVFQAKKRLLEPLGLVEEAAKTAPGKKR |
| | Olig001 | PVEQSPQEPDSSSGIGKTGQQPAKKRLNFGQTGDTESVPDPQPIGEPP |
| | (BNP61) | AAPSGVGSLTMASGGGAPVADNNEGADGVGSSSGNWHCDSQWLGDRVI |
| | capsid | TTSTRTWALPTYNNHLYKQISNGTSGGATNDNTYFGYSTPWGYFDFNR |
| | | FHCHFSPRDWQRLINNNWGFRPKRLSFKLFNIQVKEVTQNEGTKTIAN |
| | | NLTSTVQVFTDSEYQLPYVLGSAHQGCLPPFPADVFMIPQYGYLTLNN |
| | | GSQAVGRSSFYCLEYFPSQMLRTGNNFQFTYTFEDVPFHSSYAHSQSL |
| | | DRLMNPLIDQYLYYLSRTQTTGGTANTQTLGFSQGGPNTMANQAKNWL |
| | | PGPCYRQQRVSTTTGQNNNSNFAWTAGTKYHLNGRNSLANPGIAMATH |
| | | KDDKERFFPSNGILIFGKQNAARDNADYSDVMLTSEEEIKTTNPVATE |
| | | EYGIVADNLQQQNTAPQIGTVNSQGALPGMVWQNRDVYLQGPIWAKIP |
| | | HTDGNFHPSPLMGGFGLKHPPPQILIKNTPVPADPPTTFNQSKLNSFI |
| | | TQYSTGQVSVEIEWELQKENSKRWNPEIQYTSNYYKSTSVDFAVNTEG |
| | | VYSEPHPIGTRYLTRPL |
| SEQ ID NO: 15 | Amino acid | MAADGYLPDWLEDNLSEGIREWWDLKPGAPKPKANQQKQDDGRGLVLP |
| | sequence | GYKYLGPFNGLDKGEPVNAADAAALEHDKAYDQQLKAGDNPYLRYNHA |
| | for | DAEFQERLQEDTSFGGNLGRAVFQAKKRVLEPLGLVEEGAKTAPGKKR |
| | Olig002 | PVEQSPQEPDSSSGIGKTGQQPAKKRLNFGQTGDTESVPDPQPIGEPP |
| | (BNP62) | AAPSGVGSLTMASGGGAPVADNNEGADGVGSSSGNWHCDSQWLGDRVI |
| | capsid | TTSTRTWALPTYNNHLYKQISSASTGASNDNHYFGYSTPWGYFDFNRF |
| | | HCHFSPRDWQRLINNNWGFRPKRLNFKLFNIQVKEVTDNNGVKTIANN |
| | | LTSTVQVFTDSDYQLPYVLGSAHQGCLPPFPADVFMIPQYGYLTLNNG |
| | | SQAVGRSSFYCLEYFPSQMLRTGNNFQFTYTFEDVPFHSSYAHSQSLD |
| | | RLMNPLIDQYLYYLSRTQTTGGTANTQTLGFSQGGPNTMANQAKNWLP |
| | | GPCYRQQRVSTTTGQNNNSNFAWTAGTKYHLNGRNSLANPGIAMATHK |
| | | DDKERFFPSNGILIFGKQNAARDNADYSDVMLTSEEEIKTTNPVATEE |
| | | YGIVADNLQQQNTAPQIGTVNSQGALPGMVWQNRDVYLQGPIWAKIPH |
| | | TDGNFHPSPLMGGFGLKHPPPQILIKNTPVPADPPTTFNQSKLNSFIT |
| | | QYSTGQVSVEIEWELQKENSKRWNPEIQYTSNYYKSTSVDFAVNTEGV |
| | | YSEPHPIGTRYLTRPL |
| SEQ ID NO: 16 | Amino acid | MAADGYLPDWLEDTLSEGIRQWWKLKPGPPPPKPAERHKDDSRGLVLP |
| | sequence | GYKYLGPFNGLDKGEPVNEADAAALEHDKAYDRQLDSGDNPYLKYNHA |
| | for | DAEFQERLQGDTSFGGNLGRAVFQAKKRVLEPLGLVEEGAKTAPGKKR |
| | Olig003 | PVEQSPQEPDSSSGIGETGQQPAKKRLNFGQTGDSESVPDPQPLGEPP |
| | (BNP63) | ATPAAVGPTTMASGGGAPMADNNEGADGVGSSSGNWHCDSQWLGDRVI |
| | capsid | TTSTRTWALPTYNNHLYKQISSASTGASNDNHYFGYSTPWGYFDFNRF |
| | | HCHFSPRDWQRLINNNWGFRPKRLSFKLFNIQVKEVTDNNGVKTIANN |
| | | LTSTVQVFTDSEYQLPYVLGSAHQGCLPPFPADVFMIPQYGYLTLNNG |
| | | SQAVGRSSFYCLEYFPSQMLRTGNNFTFSYTFEDVPFHSSYAHSQSLD |
| | | RLMNPLIDQYLYYLSRTQTTGGTANTQTLGFSQGGPNTMANQAKNWLP |
| | | GPCYRQQRVSTTTGQNNNSNFAWTAGTKYHLNGRNSLANPGIAMATHK |
| | | DDKERFFPSNGILIFGKQNAARDNADYSDVMLTSEEEIKTTNPVATEE |
| | | YGIVADNLQQQNTAPQIGTVNSQGALPGMVWQNRDVYLQGPIWAKIPH |
| | | TDGNFHPSPLMGGFGLKHPPPQILIKNTPVPADPPTTFNQSKLNSFIT |
| | | QYSTGQVSVEIEWELQKENSKRWNPEIQYTSNYYKSTSVDFAVNTEGV |
| | | YSEPHPIGTRYLTRPL |
| SEQ ID NO: 17 | enhancer | CGTTACATAACTTACGGTAAATGGCCCGCCTGGCTGACCGCCCAACGA |
| | | CCCCCGCCCATTGACGTCAATAGTAACGCCAATAGGGACTTTCCATTG |
| | | ACGTCAATGGGTGGAGTATTTACGGTAAACTGCCCACTTGGCAGTACA |
| | | TCAAGTGTATCATATGCCAAGTACGCCCCCTATTGACGTCAATGACGG |
| | | TAAATGGCCCGCCTGGCATTGTGCCCAGTACATGACCTTATGGGACTT |
| | | TCCTACTTGGCAGTACATCTACGTATTAGTCATCGCTATTACCATG |
| SEQ ID NO: 18 | CBA exon 1 | GGAGTCGCTGCGACGCTGCCTTCGCCCCGTGCCCCGCTCCGCCGCCGC |
| | | CTCGCGCCGCCCGCCCCGGCTCTGACTGACCGCGTTACTCCCACAG |
| SEQ ID NO: 19 | 3′ ITR | CGCCCGACGCCCGGGCTTTGCCCGGGCGGCCTCAGTGAGCGAGCGAGC |
| | | GCGCCAGCTGGCGTAATAGCGAAGAGGCCCGCACCGATCGCCCTTCCC |
| | | AACAGTTGCGCAGCCTG |
| SEQ ID NO: 20 | ASPA | CTGCGCGCTCGCTCGCTCACTGAGGCCGCCCGGGCAAAGCCCGGGCGT |
| | transgene | CGGGCGACCTTTGGTCGCCCGGCCTCAGTGAGCGAGCGAGCGCGCAGA |
| | cassette | GAGGGAGTGGGGTTCGGTACCCGTTACATAACTTACGGTAAATGGCCC |
| | | GCCTGGCTGACCGCCCAACGACCCCCGCCCATTGACGTCAATAGTAAC |
| | | GCCAATAGGGACTTTCCATTGACGTCAATGGGTGGAGTATTTACGGTA |
| | | AACTGCCCACTTGGCAGTACATCAAGTGTATCATATGCCAAGTACGCC |
| | | CCCTATTGACGTCAATGACGGTAAATGGCCCGCCTGGCATTGTGCCCA |
| | | GTACATGACCTTATGGGACTTTCCTACTTGGCAGTACATCTACGTATT |
| | | AGTCATCGCTATTACCATGGTCGAGGTGAGCCCCACGTTCTGCTTCAC |
| | | TCTCCCCATCTCCCCCCCCTCCCCACCCCCAATTTTGTATTTATTTAT |
| | | TTTTTAATTATTTTGTGCAGCGATGGGGGCGGGGGGGGGGGGGGGGCG |
| | | CGCGCCAGGCGGGGCGGGGCGGGGCGAGGGGCGGGGCGGGGCGAGGCG |
| | | GAGAGGTGCGGCGGCAGCCAATCAGAGCGGCGCGCTCCGAAAGTTTCC |
| | | TTTTATGGCGAGGCGGCGGCGGCGGCGGCCCTATAAAAAGCGAAGCGC |
| | | GCGGCGGGCGGGAGTCGCTGCGACGCTGCCTTCGCCCCGTGCCCCGCT |
| | | CCGCCGCCGCCTCGCGCCGCCCGCCCCGGCTCTGACTGACCGCGTTAC |
| | | TCCCACAGGTGAGCGGGCGGGACGGCCCTTCTCCTCCGGGCTGTAATT |
| | | AGCTGAGCAAGAGGTAAGGGTTTAAGGGATGGTTGGTTGGTGGGGTAT |
| | | TAATGTTTAATTACCTGGAGCACCTGCCTGAAATCACTTTTTTTCAGG |
| | | TTGGACCGGTATGACCTCCTGTCATATAGCCGAGGAGCACATCCAGAA |
| | | AGTGGCCATTTTCGGCGGGACACACGGAAACGAACTTACAGGAGTGTT |
| | | TCTGGTGAAACACTGGCTTGAAAATGGTGCGGAGATCCAAAGGACCGG |
| | | CCTGGAGGTCAAACCTTTTATTACAAATCCCCGGGCGGTCAAGAAGTG |
| | | CACACGGTACATTGATTGTGATCTTAATCGCATATTCGACCTGGAGAA |
| | | CCTTGGGAAGAAAATGTCTGAAGATCTGCCCTACGAAGTGAGGCGAGC |
| | | ACAAGAGATAAACCACCTGTTCGGACCGAAAGACAGTGAAGACTCCTA |
| | | TGACATCATTTTCGACCTGCACAACACTACGAGTAACATGGGGTGTAC |
| | | CCTGATCCTCGAAGACTCCCGAAACAATTTCCTGATACAGATGTTTCA |
| | | TTACATCAAAACTAGTCTGGCCCCTCTCCCCTGCTACGTTTATCTGAT |
| | | CGAACACCCTTCTCTCAAATACGCTACCACCCGCTCTATTGCTAAGTA |
| | | CCCCGTCGGGATCGAGGTCGGCCCACAACCTCAAGGTGTGCTCCGGGC |
| | | CGATATTTTGGACCAGATGAGAAAGATGATTAAACACGCTCTCGACTT |
| | | CATTCACCACTTTAACGAGGGGAAGGAATTTCCCCCTTGTGCCATCGA |
| | | GGTTTATAAGATTATCGAGAAGGTGGACTACCCAAGAGACGAAAACGG |
| | | GGAGATAGCTGCCATCATCCACCCTAATTTGCAAGATCAGGACTGGAA |
| | | GCCCCTGCACCCAGGAGACCCCATGTTTCTGACCTTGGATGGAAAGAC |
| | | GATCCCCCTGGGCGGTGATTGTACAGTGTACCCAGTCTTTGTCAACGA |
| | | GGCCGCTTACTATGAGAAAAAGGAGGCTTTTGCAAAGACAACAAAGCT |
| | | CACTTTGAATGCAAAGTCCATCAGGTGCTGTCTGCACTAAGCGGCCGC |
| | | GGGGATCCCTCGACTGTGCCTTCTAGTTGCCAGCCATCTGTTGTTTGC |
| | | CCCTCCCCCGTGCCTTCCTTGACCCTGGAAGGTGCCACTCCCACTGTC |
| | | CTTTCCTAATAAAATGAGGAAATTGCATCGCATTGTCTGAGTAGGTGT |
| | | CATTCTATTCTGGGGGGTGGGGTGGGGCAGGACAGCAAGGGGGAGGAT |
| | | TGGGAAGACAACAGCAGGCATGCTGGGGATGCGGTGGGCTCTATGGCT |
| | | TCTGAGGCGGAAAGAACCAGCTTTGGACGCGTAGGAACCCCTAGTGAT |
| | | GGAGTTGGCCACTCCCTCTCTGCGCGCTCGCTCGCTCACTGAGGCCGG |
| | | GCGACCAAAGGTCGCCCGACGCCCGGGCTTTGCCCGGGCGGCCTCAGT |
| | | GAGCGAGCGAGCGCGCCAGCTGGCGTAATAGCGAAGAGGCCCGCACCG |
| | | ATCGCCCTTCCCAACAGTTGCGCAGCCTG |

1. An isolated or modified nucleic acid encoding aspartoacyltransferase (ASPA) comprising a nucleic acid sequence at least about 80%, 85%, 90%, 95%, 98%, 99% or 100% identical to the nucleic acid sequence of SEQ ID NO:2.

2. (canceled)

3. A vector genome comprising the modified nucleic acid of claim 1.

4. The vector genome of claim 3, wherein the vector genome is a recombinant adeno-associated virus (rAAV) vector genome.

5. (canceled)

6. A recombinant adeno-associated virus (rAAV) vector comprising the vector genome of claim 3 and a capsid selected from the group consisting of a capsid of Olig001, Olig002, Olig003, AAV1, AAV2, AAV3, AAV3A, AAV3B, AAV4, AAV5, AAV6, AAV7, AAV8, AAV9, AAV10, AAV11, AAV12, AAVrh10, AAVrh74, RHM4-1, RHM15-1, RHM15-2, RHM15-3/RHM15-5, RHM15-4, RHM15-6, AAVhu.26, AAV1.1, AAV2.5, AAV6.1, AAV6.3.1, AAV9.45, AAV2i8, AAV2G9, AAV2i8G9, AAV2-TT, AAV2-TT-S312N, AAV3B-S312N, AAV-DJ, AAV-DJ/8, AAV-DJ/9 and AAV-LK03.

7. The rAAV vector of claim 6, wherein the capsid is an Olig001, an Olig002 or an Olig003 capsid.

8. The rAAV vector of claim 6, wherein the capsid is an Olig001 capsid comprising a viral protein 1(VP1) and wherein the VP1 comprises an amino acid sequence at least about 70%, 75%, 80%, 85%, 90%, 95%, 98%, 99% or 100% identical to the amino acid sequence of SEQ ID NO:14.

9-10. (canceled)

11. The rAAV vector of claim 6, wherein the vector genome further comprises at least one element selected from the group consisting of at least one AAV inverted terminal repeat (ITR) sequence, an enhancer, a promoter, an exon, an intron, and a poly-adenylation (polyA) signal sequence.

12. The rAAV vector of claim 6, wherein the vector genome further comprises at least one element selected from the group consisting of at least one AAV2 ITR, a cytomegalovirus (CMV) enhancer, a hybrid form of the CBA promoter (CBh promoter), a chicken B-actin (CBA) exon, a CBA intron, a minute virus of mice (MVM) intron and a bovine growth hormone (BGH) polyA.

13. The rAAV vector of claim 6, wherein the vector genome further comprises a least one element selected from the group consisting of at least one ITR comprising the nucleic acid sequence of SEQ ID NO:5, SEQ ID NO:12 or SEQ ID NO:19, an enhancer comprising the nucleic acid sequence of SEQ ID NO:6 or SEQ ID NO:17, a promoter comprising the nucleic acid sequence of SEQ ID NO:7, an exon comprising the nucleic acid sequence of SEQ ID NO:8 or SEQ ID NO:18, an intron comprising the nucleic acid sequence of SEQ ID NO:9, an intron comprising the nucleic acid sequence of SEQ ID NO:10 and a polyA comprising the nucleic acid sequence of SEQ ID NO:11.

14. An rAAV vector comprising a vector genome comprising from 5′ to 3′:
a) an AAV inverted terminal repeat (ITR) comprising the nucleic acid sequence of SEQ ID NO:5, SEQ ID NO:12 or SEQ ID NO:19;
b) an enhancer comprising the nucleic acid sequence of SEQ ID NO:6 or SEQ ID NO:17;
c) a promoter comprising the nucleic acid sequence of SEQ ID NO:7;
d) an exon comprising the nucleic acid sequence of SEQ ID NO:8 or SEQ ID NO:18;
e) an intron comprising the nucleic acid sequence of SEQ ID NO:9;
f) an intron comprising the nucleic acid sequence of SEQ ID NO:10;
g) a modified nucleic acid encoding aspartoacyltransferase (ASPA) comprising the nucleic acid sequence of SEQ ID NO:2
h) a polyA comprising the nucleic acid sequence of SEQ ID NO:11; and
i) an AAV ITR comprising the nucleic acid sequence of SEQ ID NO:5, SEQ ID NO:12 or SEQ ID NO:19.

15-17. (canceled)

18. A pharmaceutical composition comprising the rAAV vector of claim 6.

19. A method of treating and/or preventing a disease, disorder or condition associated with deficiency or dysfunction of ASPA, the method comprising administering a therapeutically effective amount of the rAAV vector of claim 6.

20. The method of claim 19, wherein the disease, disorder or condition associated with deficiency or dysfunction of ASPA is Canavan disease.

21. The method of claim 19, wherein the rAAV vector is administered directly to the brain and/or central nervous system.

22. The method of claim 19, wherein the rAAV vector is administered to a region of the central nervous system selected from the group consisting of brain parenchyma, spinal canal, subarachnoid space, a ventricle of the brain, cisterna magna and a combination thereof,
or wherein the rAAV vector is administered by a method selected from the group consisting of intraparenchymal administration, intrathecal administration, intracerebroventricular administration, intracisternal magna administration and a combination thereof.

23. (canceled)

24. A host cell comprising the isolated nucleic acid of claim 1.

25. The host cell of claim 24, wherein the cell is selected from the group consisting of VERO, WI38, MRCS, A549, HEK293, B-50 or any other HeLa cell, HepG2, Saos-2, HuH7, and HT1080.

26-27. (canceled)

28. The host cell of claim 25, wherein the cell comprises at least one nucleic acid encoding at least one protein selected from the group consisting of an AAV rep protein, an AAV capsid (Cap) protein, an adenovirus (Ad) early region 1A (Ela) protein, an Ad E1b protein, an Ad E2a protein, an Ad E4 protein and a viral associated (VA) RNA.

29. A kit for the treatment of Canavan disease (CD), comprising a therapeutically effective amount of an isolated nucleic acid of claim 1.

30-34. (canceled)

35. A method of determining biodistribution of a transgene in the brain of a subject wherein the transgene is expressed from an rAAV vector comprising an Olig001 capsid, the method comprising
a) administration of the rAAV vector to the subject by intracrebroventricular (ICV) injection or by intraparenchymal (IP) injection;
b) fixation of the brain;
c) electrophoretic clearing of the brain;
d) 3D microscopic imaging of a brain tissue section;
e) quantification of transgene expression.

36-40. (canceled)


SEQUENCE LISTING (20 sequences)

SEQ ID NO: 1 - DNA, 942 bp, Artificial
  Synthetic
atgacctcctgtcatatagccgaggagcacatccagaaagtggccattttcggcgggacacatgggaacgagctgactggcgttttcctggtcaagcactggctcgaaaatggcgcggaaattcagagaacgggcctggaggtcaaaccttttattactaacccccgcgcggtgaagaaatgtacccggtacatcgactgcgatcttaaccgaatctttgatctggaaaatctgggaaaaaaaatgagcgaggacctgccctacgaagtccgcagagcacaggagattaatcatctcttcggacccaaggactccgaggacagctacgatatcatcttcgacttgcacaatactacttccaatatgggatgtaccttgatactggaggactcacgaaataacttcttgattcagatgttccattacatcaaaacctctctcgctcctctcccttgctacgtatatttgatcgagcaccctagtctgaaatatgccactacacgaagcatagctaagtatcccgttggtattgaggtgggcccccagccccagggagtgctgcgggctgacatccttgaccagatgagaaaaatgatcaaacacgcccttgacttcatccaccactttaatgaaggcaaagagtttcctccctgtgccatagaggtgtataaaatcatcgaaaaagttgactatccacgggatgagaacggcgagatcgctgccatcatccatcccaatttgcaagatcaggattggaaacctttgcacccaggcgaccctatgttcctgacattggatggcaagaccatacccctgggtggtgattgcactgtgtacccagttttcgtaaacgaggcagcgtactatgaaaagaaagaggcatttgcaaaaaccactaagttgacactgaatgccaagagcattagatgctgtcttcattaa

SEQ ID NO: 2 - DNA, 942 bp, Artificial
  Synthetic
atgacctcctgtcatatagccgaggagcacatccagaaagtggccattttcggcgggacacacggaaacgaacttacaggagtgtttctggtgaaacactggcttgaaaatggtgcggagatccaaaggaccggcctggaggtcaaaccttttattacaaatccccgggcggtcaagaagtgcacacggtacattgattgtgatcttaatcgcatattcgacctggagaaccttgggaagaaaatgtctgaagatctgccctacgaagtgaggcgagcacaagagataaaccacctgttcggaccgaaagacagtgaagactcctatgacatcattttcgacctgcacaacactacgagtaacatggggtgtaccctgatcctcgaagactcccgaaacaatttcctgatacagatgtttcattacatcaaaactagtctggcccctctcccctgctacgtttatctgatcgaacacccttctctcaaatacgctaccacccgctctattgctaagtaccccgtcgggatcgaggtcggcccacaacctcaaggtgtgctccgggccgatattttggaccagatgagaaagatgattaaacacgctctcgacttcattcaccactttaacgaggggaaggaatttcccccttgtgccatcgaggtttataagattatcgagaaggtggactacccaagagacgaaaacggggagatagctgccatcatccaccctaatttgcaagatcaggactggaagcccctgcacccaggagaccccatgtttctgaccttggatggaaagacgatccccctgggcggtgattgtacagtgtacccagtctttgtcaacgaggccgcttactatgagaaaaaggaggcttttgcaaagacaacaaagctcactttgaatgcaaagtccatcaggtgctgtctgcactaa

SEQ ID NO: 3 - DNA, 942 bp, Homo sapiens
atgacttcttgtcacattgctgaagaacatatacaaaaggttgctatctttggaggaacccatgggaatgagctaaccggagtatttctggttaagcattggctagagaatggcgctgagattcagagaacagggctggaggtaaaaccatttattactaaccccagagcagtgaagaagtgtaccagatatattgactgtgacctgaatcgcatttttgaccttgaaaatcttggcaaaaaaatgtcagaagatttgccatatgaagtgagaagggctcaagaaataaatcatttatttggtccaaaagacagtgaagattcctatgacattatttttgaccttcacaacaccacctctaacatggggtgcactcttattcttgaggattccaggaataactttttaattcagatgtttcattacattaagacttctctggctccactaccctgctacgtttatctgattgagcatccttccctcaaatatgcgaccactcgttccatagccaagtatcctgtgggtatagaagttggtcctcagcctcaaggggttctgagagctgatatcttggatcaaatgagaaaaatgattaaacatgctcttgattttatacatcatttcaatgaaggaaaagaatttcctccctgcgccattgaggtctataaaattatagagaaagttgattacccccgggatgaaaatggagaaattgctgctatcatccatcctaatctgcaggatcaagactggaaaccactgcatcctggggatcccatgtttttaactcttgatgggaagacgatcccactgggcggagactgtaccgtgtaccccgtgtttgtgaatgaggccgcatattacgaaaagaaagaagcttttgcaaagacaactaaactaacgctcaatgcaaaaagtattcgctgctgtttacattag

SEQ ID NO: 4 - protein, 313 aa, Homo sapiens
MTSCHIAEEHIQKVAIFGGTHGNELTGVFLVKHWLENGAEIQRTGLEVKPFITNPRAVKKCTRYIDCDLNRIFDLENLGKKMSEDLPYEVRRAQEINHLFGPKDSEDSYDIIFDLHNTTSNMGCTLILEDSRNNFLIQMFHYIKTSLAPLPCYVYLIEHPSLKYATTRSIAKYPVGIEVGPQPQGVLRADILDQMRKMIKHALDFIHHFNEGKEFPPCAIEVYKIIEKVDYPRDENGEIAAIIHPNLQDQDWKPLHPGDPMFLTLDGKTIPLGGDCTVYPVFVNEAAYYEKKEAFAKTTKLTLNAKSIRCCLH

SEQ ID NO: 5 - DNA, 106 bp, Artificial
  Synthetic
ctgcgcgctcgctcgctcactgaggccgcccgggcaaagcccgggcgtcgggcgacctttggtcgcccggcctcagtgagcgagcgagcgcgcagagagggagtgg

SEQ ID NO: 6 - DNA, 284 bp, Artificial
  Synthetic
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcatttgcccagtacatgaccttatgggactttcctacttggcagtacatctacgtattagtcatcgctattaccat

SEQ ID NO: 7 - DNA, 278 bp, Artificial
  Synthetic
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 8 - DNA, 82 bp, Artificial
  Synthetic
ggagtcgctgcgcgctgccttcgccccgtgccccgctccgccgccgcctcgcgccgcccgccccggctctgactgaccgcgt

SEQ ID NO: 9 - DNA, 43 bp, Artificial
  Synthetic
gtgagcgggcgggacggcccttctcctccgggctgtaattagc

SEQ ID NO: 10 - DNA, 92 bp, Artificial
  Synthetic
aagaggtaagggtttaagggatggttggttggtggggtattaatgtttaattacctggagcacctgcctgaaatcactttttttcaggttgg

SEQ ID NO: 11 - DNA, 225 bp, Artificial
  Synthetic
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatgg

SEQ ID NO: 12 - DNA, 113 bp, Artificial
  Synthetic
tcgcccgacgcccgggctttgcccgggcggcctcagtgagcgagcgagcgcgcagctggcgtaatagcgaagaggcccgcaccgatcgcccttcccaacagttgcgcagcctg

SEQ ID NO: 13 - DNA, 2214 bp, Artificial
  Synthetic
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctcctcgggcatcggcaagacaggccagcagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataaccttaccagcacggtccaggtcttcacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacaaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtttggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccaccccattggcacccgttacctcacccgtcccctgtaa

SEQ ID NO: 14 - protein, 737 aa, Artificial
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEQSPQEPDSSSGIGKTGQQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 15 - protein, 736 aa, Artificial
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 16 - protein, 736 aa, Artificial
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLQGDTSFGGNLGRAVFQAKKRVLEPLGLVEEGAKTAPGKKRPVEQSPQEPDSSSGIGETGQQPAKKRLNFGQTGDSESVPDPQPLGEPPATPAAVGPTTMASGGGAPMADNNEGADGVGSSSGNWHCDSQWLGDRVITTSTRTWALPTYNNHLYKQISSASTGASNDNHYFGYSTPWGYFDFNRFHCHFSPRDWQRLINNNWGFRPKRLSFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 17 - DNA, 286 bp, Artificial
  Synthetic
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatg

SEQ ID NO: 18 - DNA, 94 bp, Artificial
  Synthetic
ggagtcgctgcgacgctgccttcgccccgtgccccgctccgccgccgcctcgcgccgcccgccccggctctgactgaccgcgttactcccacag

SEQ ID NO: 19 - DNA, 113 bp, Artificial
  Synthetic
cgcccgacgcccgggctttgcccgggcggcctcagtgagcgagcgagcgcgccagctggcgtaatagcgaagaggcccgcaccgatcgcccttcccaacagttgcgcagcctg

SEQ ID NO: 20 - DNA, 2333 bp, Artificial
  Synthetic
ctgcgcgctcgctcgctcactgaggccgcccgggcaaagcccgggcgtcgggcgacctttggtcgcccggcctcagtgagcgagcgagcgcgcagagagggagtggggttcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtatgacctcctgtcatatagccgaggagcacatccagaaagtggccattttcggcgggacacacggaaacgaacttacaggagtgtttctggtgaaacactggcttgaaaatggtgcggagatccaaaggaccggcctggaggtcaaaccttttattacaaatccccgggcggtcaagaagtgcacacggtacattgattgtgatcttaatcgcatattcgacctggagaaccttgggaagaaaatgtctgaagatctgccctacgaagtgaggcgagcacaagagataaaccacctgttcggaccgaaagacagtgaagactcctatgacatcattttcgacctgcacaacactacgagtaacatggggtgtaccctgatcctcgaagactcccgaaacaatttcctgatacagatgtttcattacatcaaaactagtctggcccctctcccctgctacgtttatctgatcgaacacccttctctcaaatacgctaccacccgctctattgctaagtaccccgtcgggatcgaggtcggcccacaacctcaaggtgtgctccgggccgatattttggaccagatgagaaagatgattaaacacgctctcgacttcattcaccactttaacgaggggaaggaatttcccccttgtgccatcgaggtttataagattatcgagaaggtggactacccaagagacgaaaacggggagatagctgccatcatccaccctaatttgcaagatcaggactggaagcccctgcacccaggagaccccatgtttctgaccttggatggaaagacgatccccctgggcggtgattgtacagtgtacccagtctttgtcaacgaggccgcttactatgagaaaaaggaggcttttgcaaagacaacaaagctcactttgaatgcaaagtccatcaggtgctgtctgcactaagcggccgcggggatccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagctttggacgcgtaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctg